(19) United States
(12) Patent Application Publication
(10) Pub. No.: US 2001/0049432 A1
(43) Pub. Date: Dec. 6, 2001

(54) Human semaphorin ZSMF-16

(76) Inventors: James L. Holloway, Seattle, WA (US); Kevin P. Foley, Cambridge, MA (US)

Correspondence Address: Jennifer K. Johnson, J.D., ZymoGenetics, Inc., 1201 Eastlake Avenue East, Seattle, WA, 98102, US

(21) Appl. No.: 09/731,179

(22) Filed: Dec. 6, 2000

Related U.S. Application Data
(60) Provisional application No. 60/169,238, filed on Dec. 6, 1999.

(51) Int. Cl. C12Q 1/68; C07H 21/04; C12P 21/04; C07K 14/435; C07K 16/18

(52) U.S. Cl. 530/350; 435/69.7; 435/325; 536/23.5; 530/388.23; 435/6

(57) ABSTRACT

Semaphorin polypeptides, polynucleotides encoding the polypeptides, and related compositions and methods are disclosed. The polypeptide is expressed in breast cancer, and neuronal tissues. The polypeptides, and polynucleotides encoding them, may be used for detecting human chromosomal abnormalities and cancers. The polypeptides may be used within methods for detecting receptors that mediate neurite outgrowth, modulate cellular proliferation and/or differentiation, and immune response. The present invention also includes antibodies to the ZSMF-16 polypeptides and uses therefore.

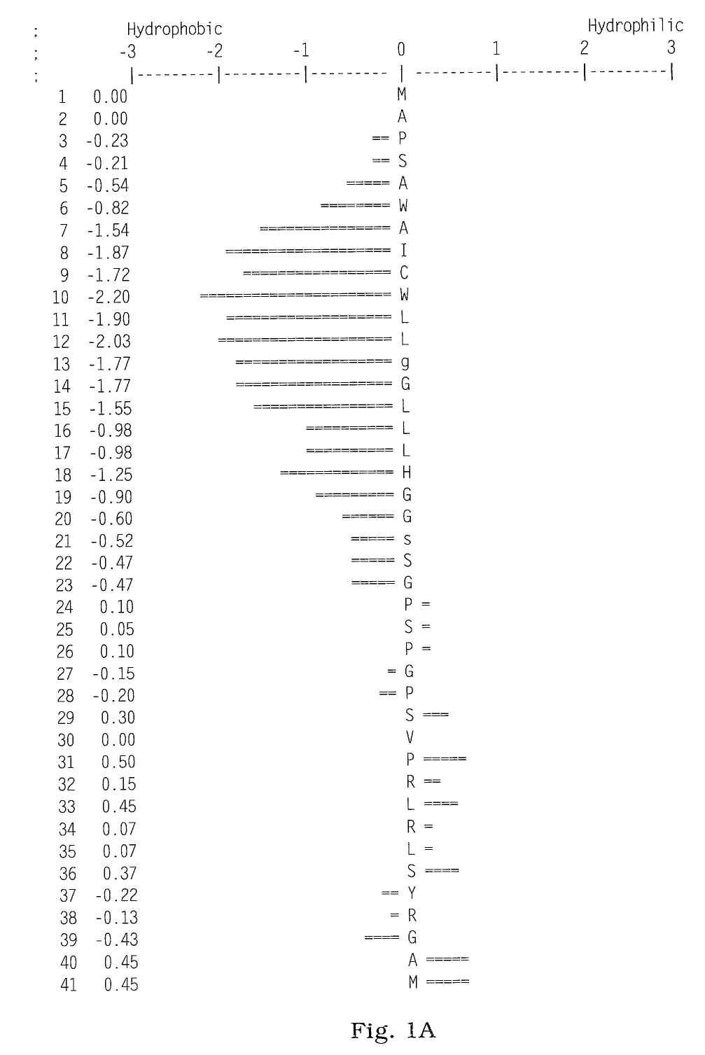

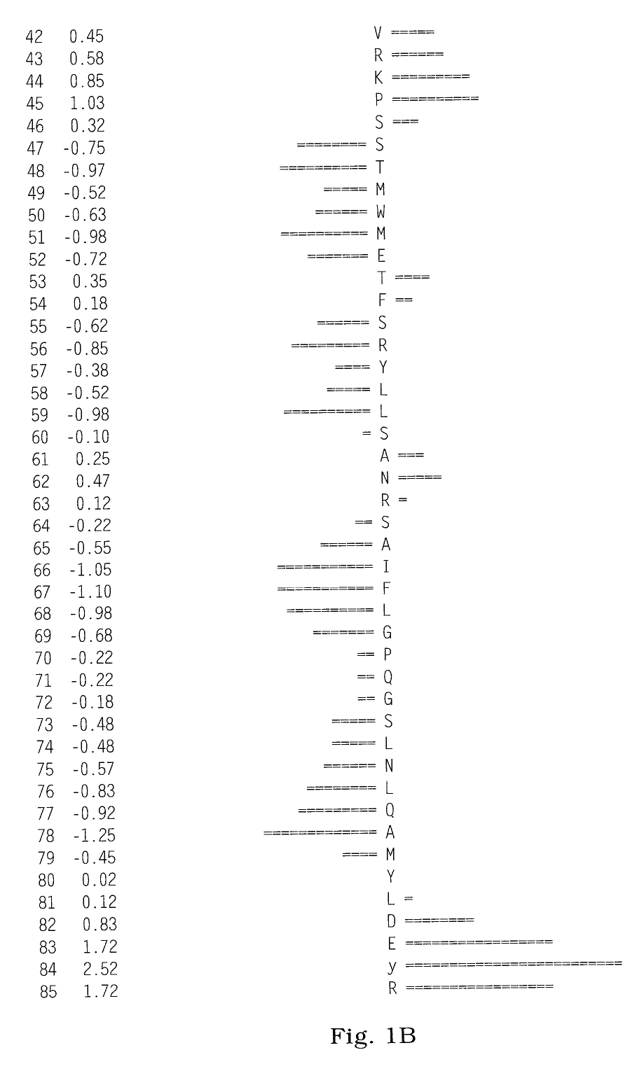

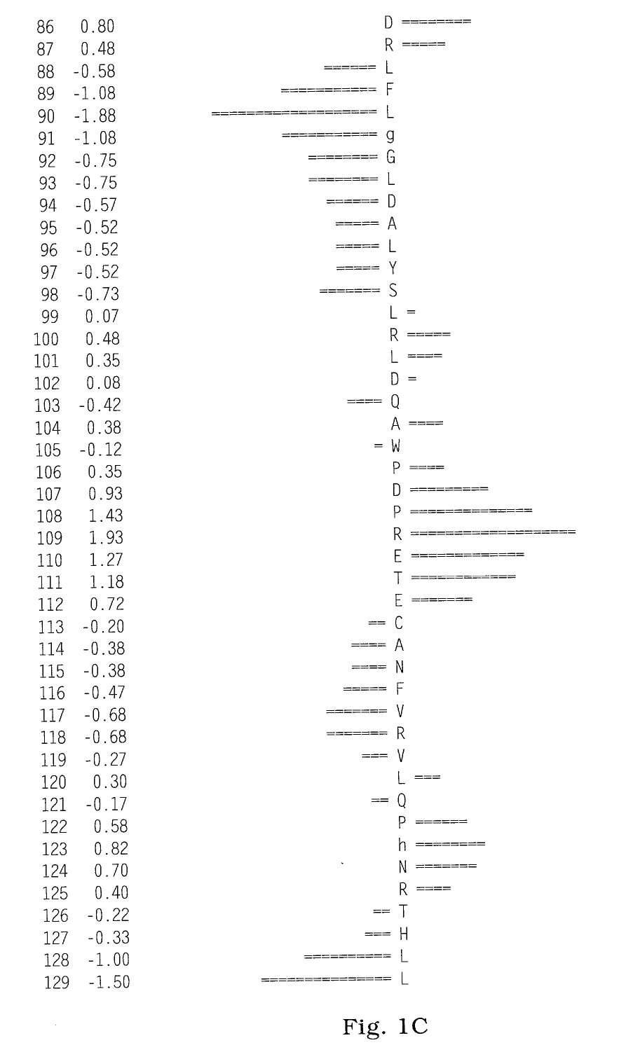

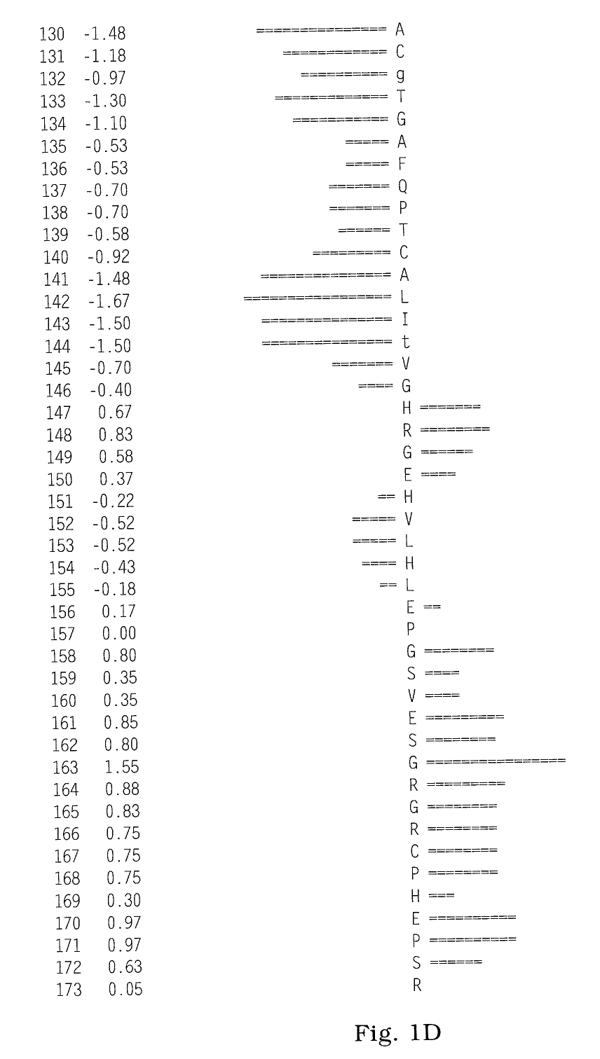

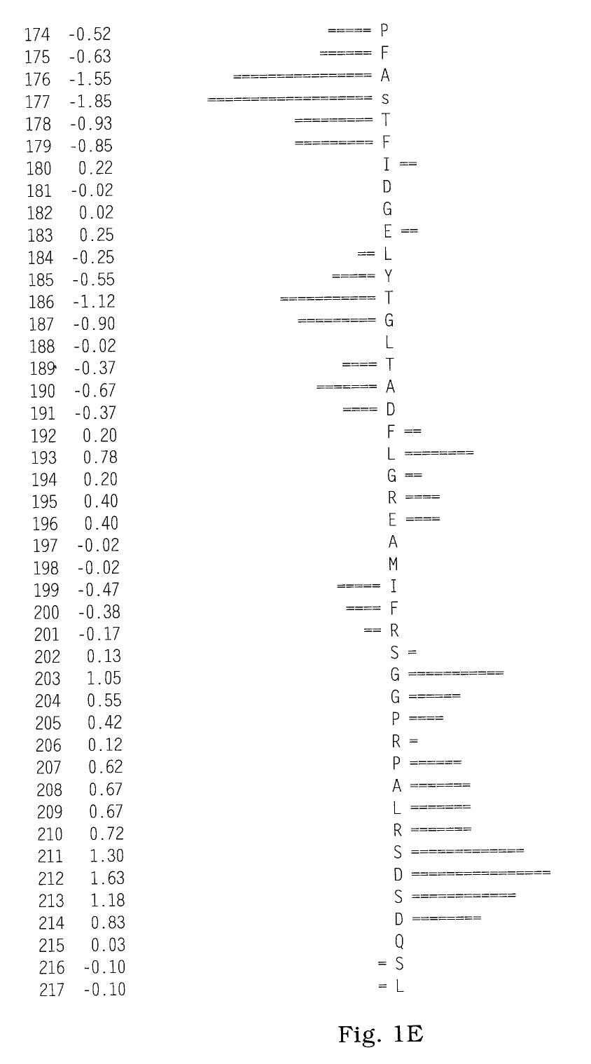

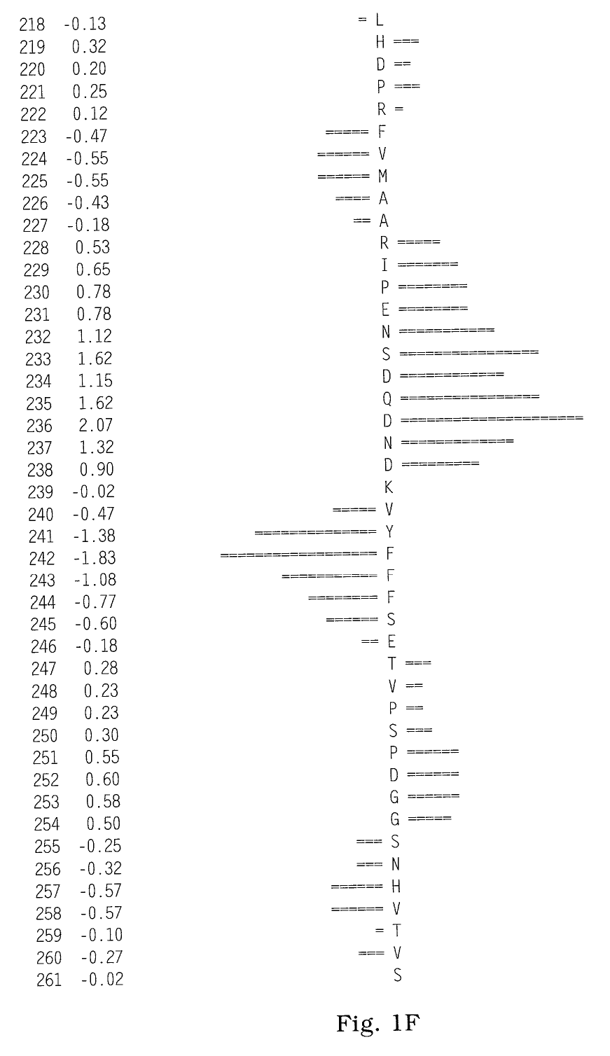

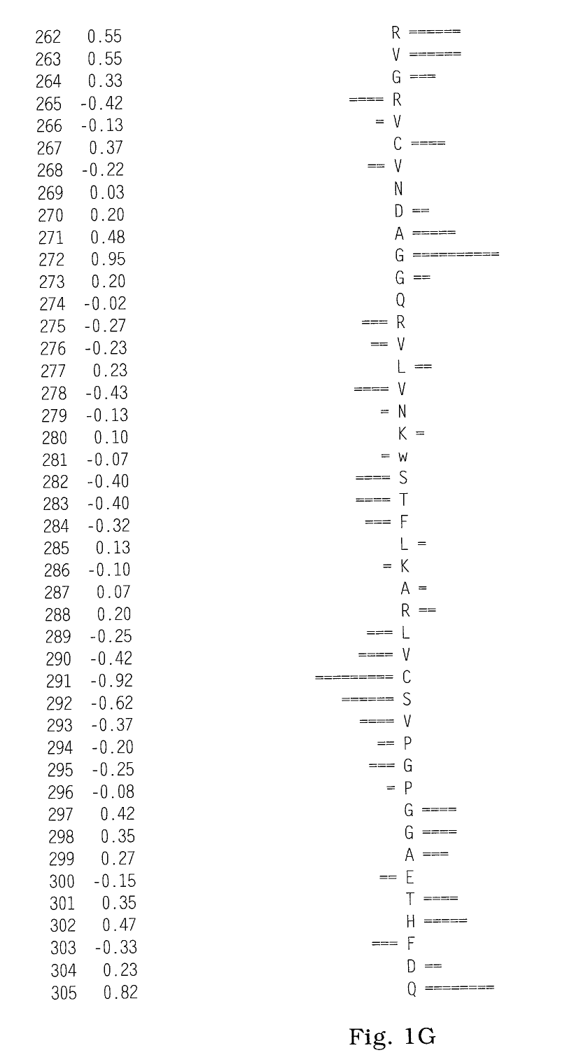

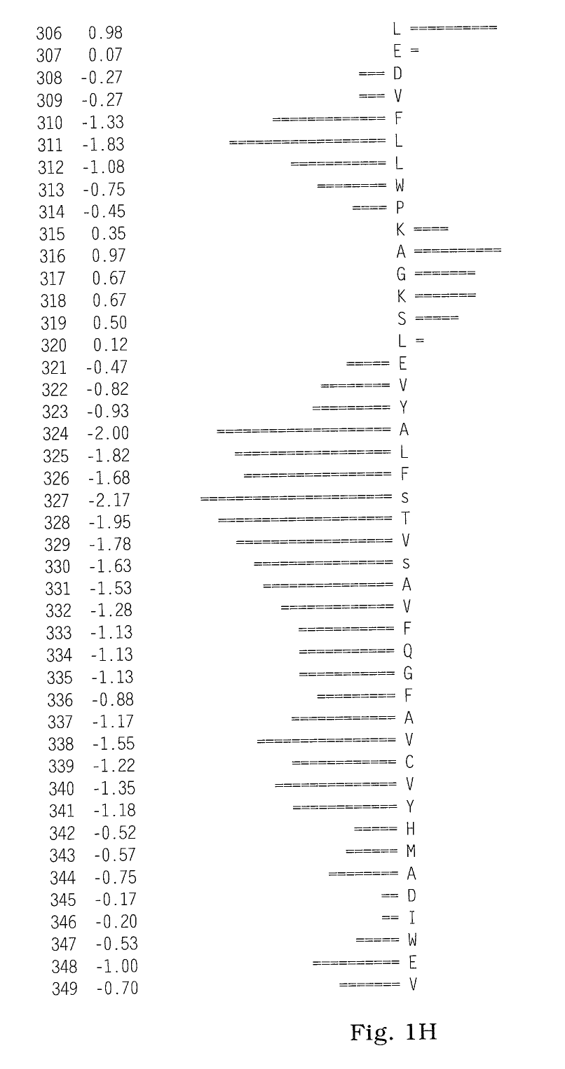

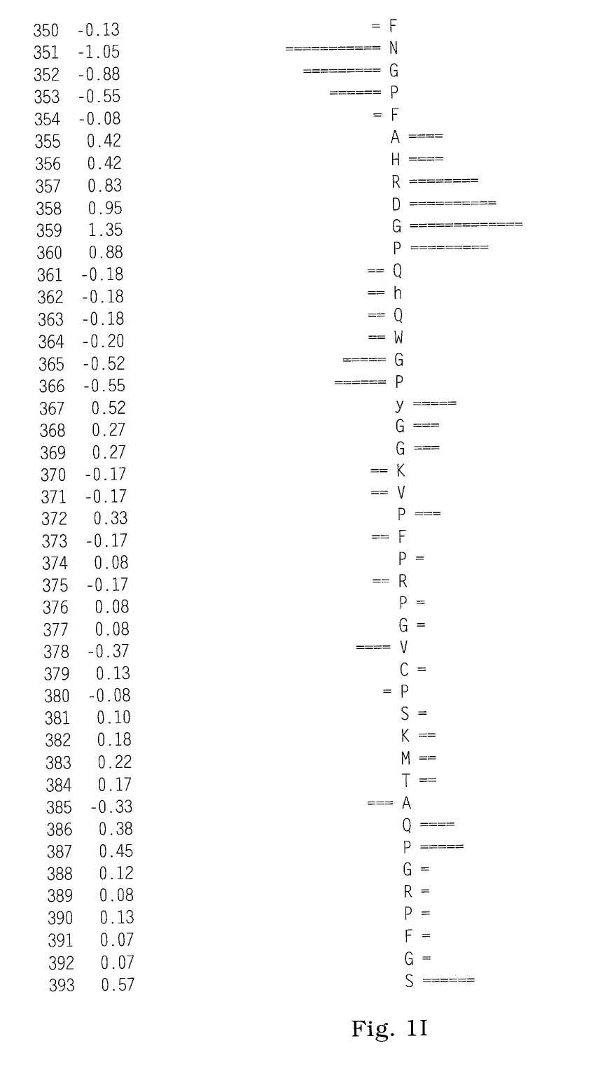

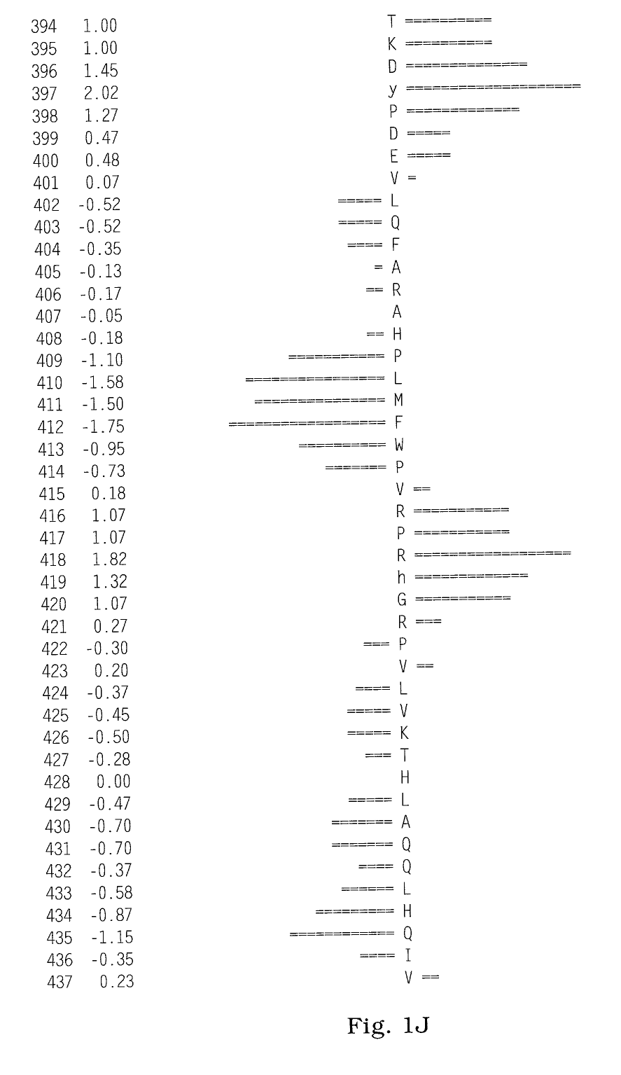

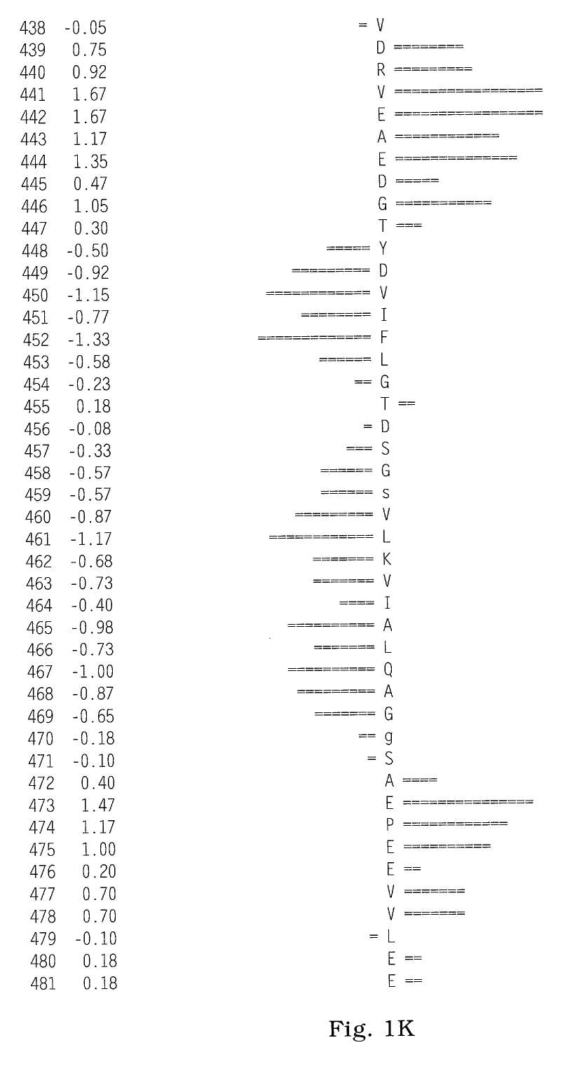

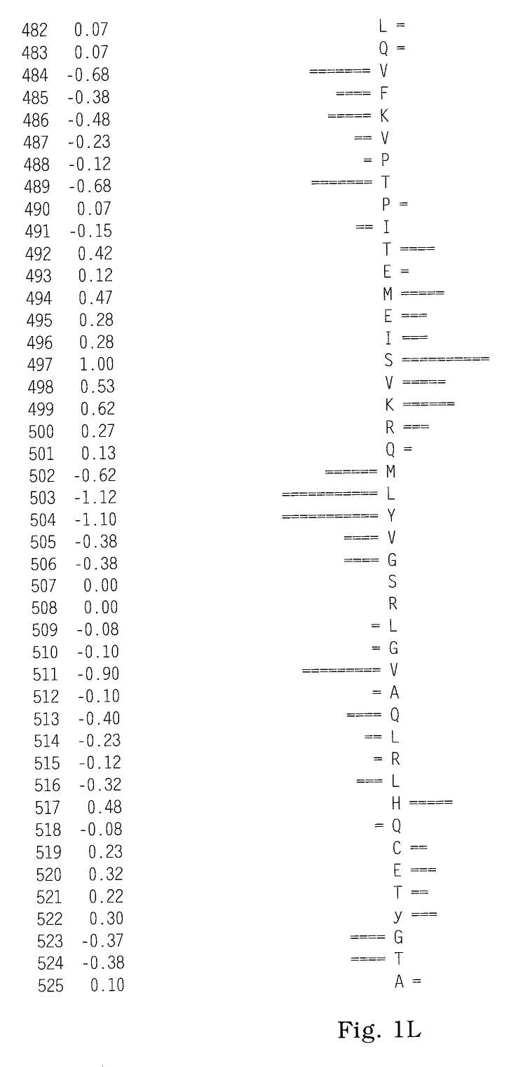

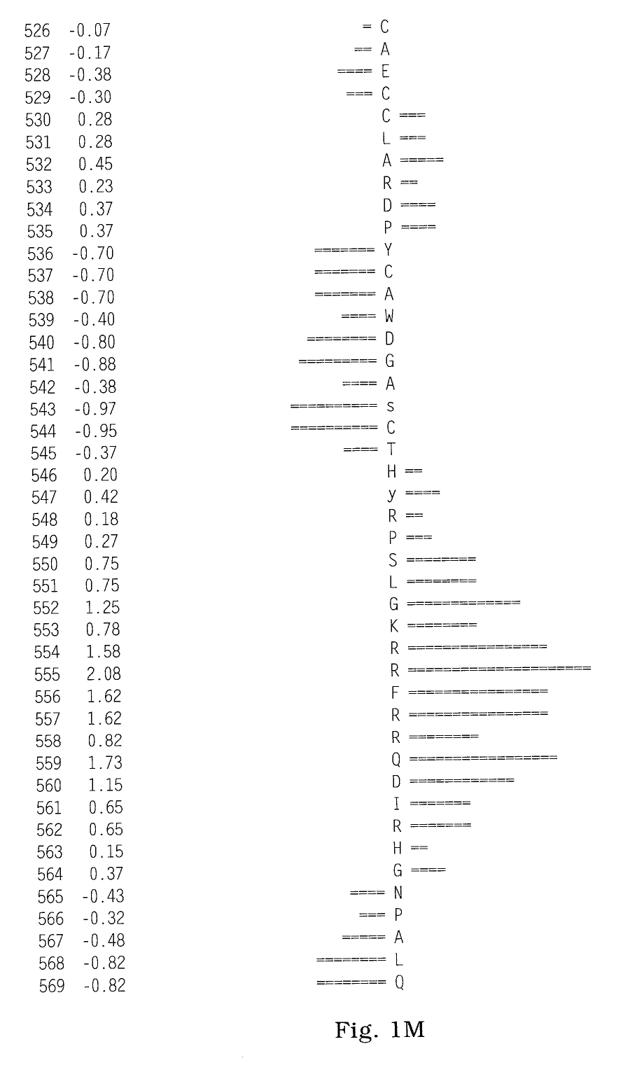

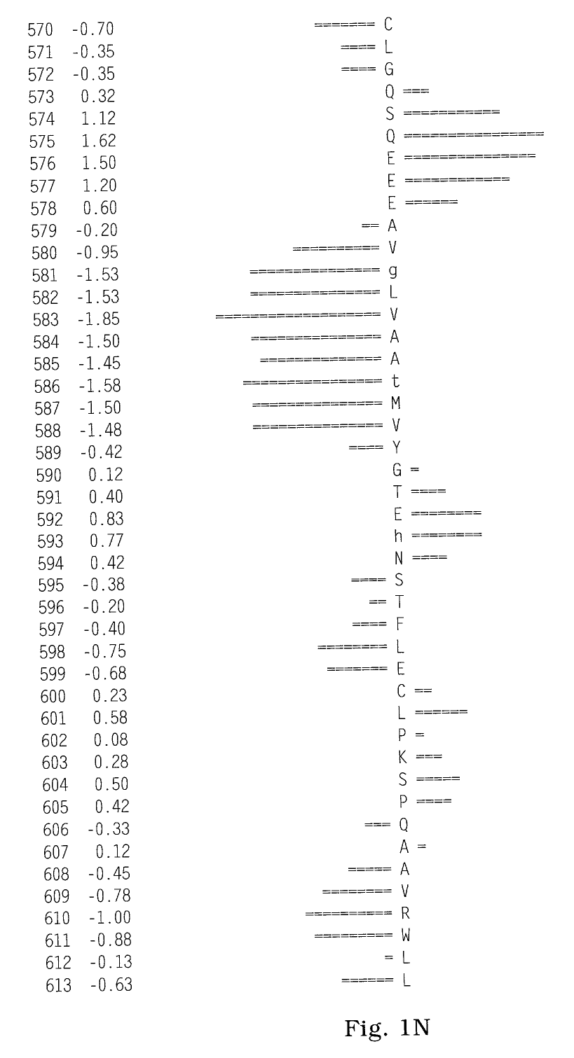

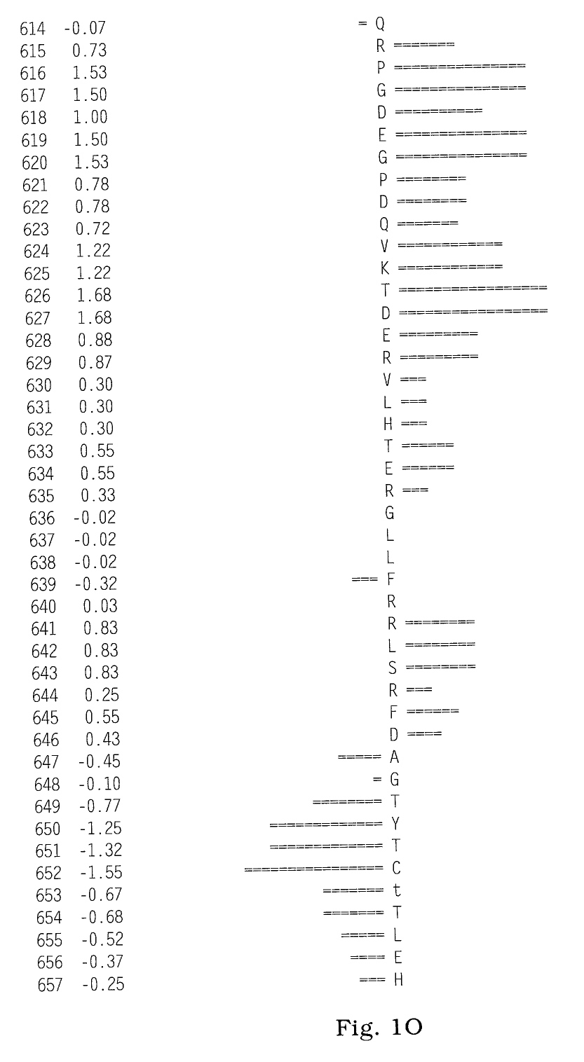

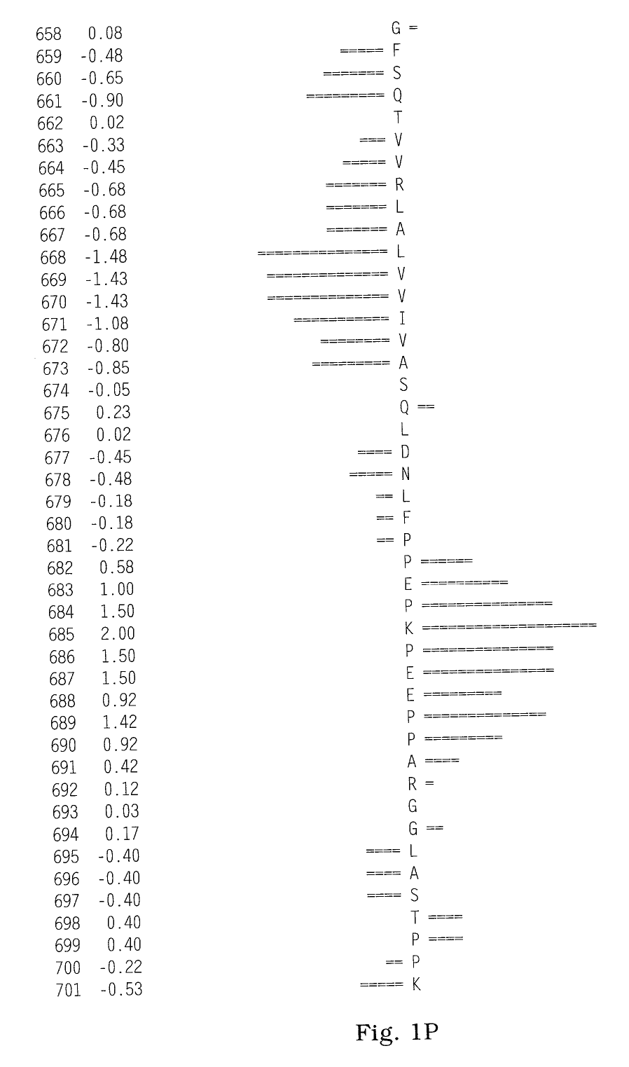

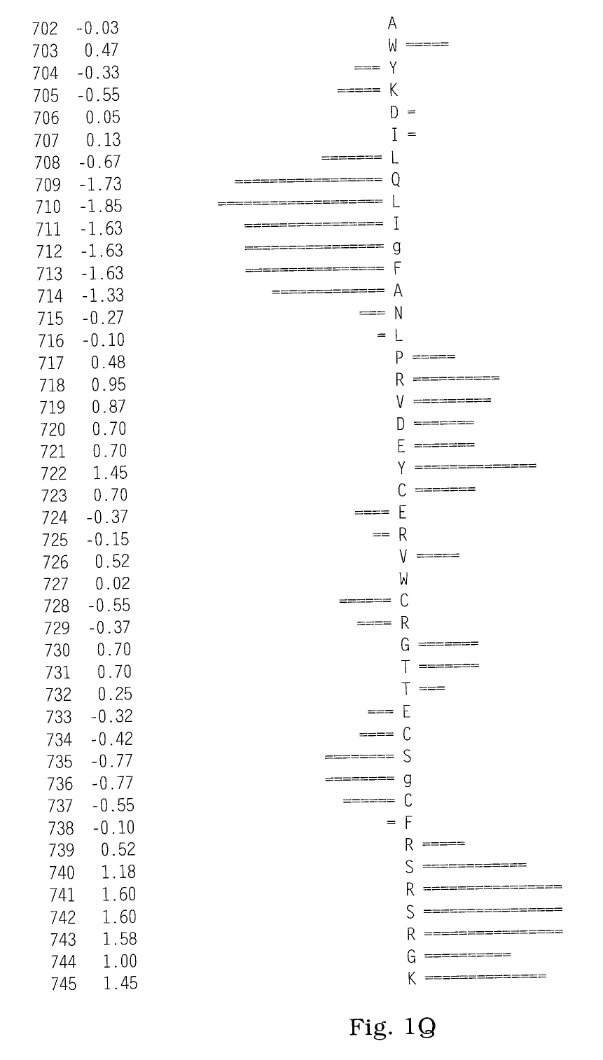

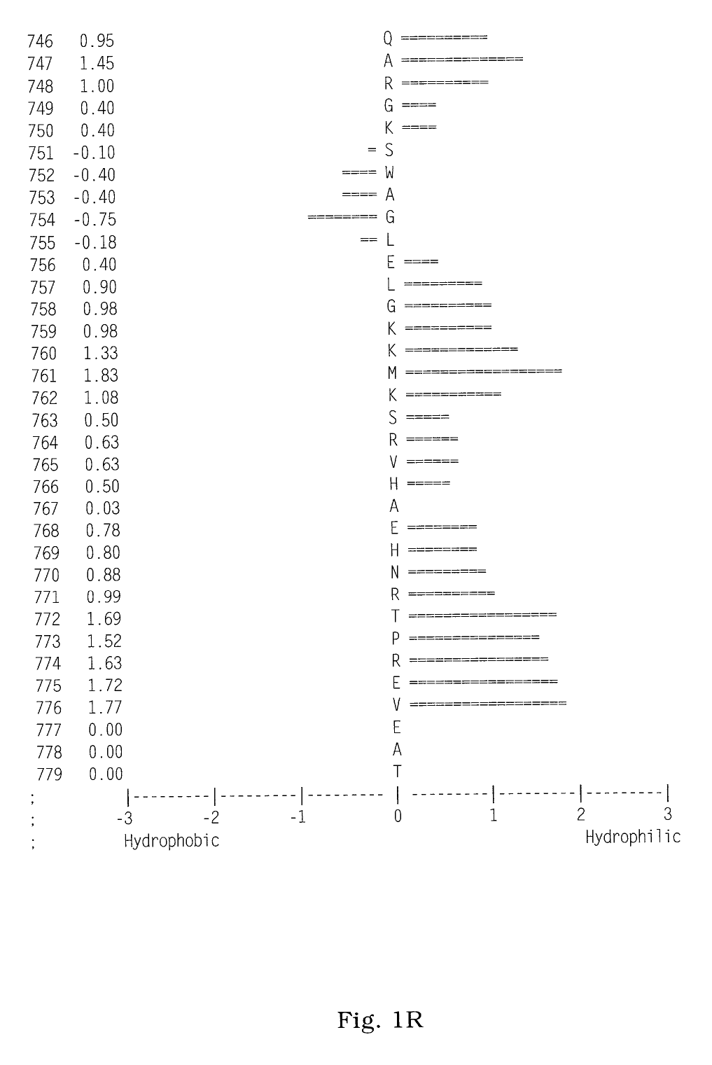

### REFERENCE TO RELATED APPLICATION

This application is related to U.S. Provisional Application No. 60/169,238, filed on Dec. 6, 1999. Under 35 U.S.C. § 119(e)(1), this application claims benefit of said Provisional Application.

### BACKGROUND OF THE INVENTION

Neuronal cell outgrowths, known as processes, grow away from the cell body to form synaptic connections. Long, thin processes that carry information away from the cell body are called axons, and short, thicker processes that carry information to and from the cell body are called dendrites. Axons and dendrites are collectively referred to as neurites. Neurites are extended by means of growth cones, the growing tip of the neurite, which is highly motile and is ultimately responsible for increasing and extending the neuronal network in the body. The growth cones are able to navigate their way to their targets using environmental cues or signals, which encourage or discourage the growth cone from extending the neurite in a particular direction. Such cues and signals include older neurons and orienting glial fibers, chemicals such as nerve growth factor released by astrocytes and other attracting or repelling substances released by target cells. The membrane of the growth cone bears molecules such as nerve cell adhesion molecule (N-CAM) which are attracted or repelled by environmental cues and thus influence the direction and degree of neurite growth. The growth cone also engulfs molecules from the environment which are transported to the cell body and influence growth. A number of proteins from vertebrates and invertebrates have been identified as influencing the guidance of neurite growth, either through repulsion or chemoattraction. Among those molecules are netrins, EPH-related receptor tyrosine kinases and their ligands, vitronectin, thrombospondin, human neuronal attachment factor-1 (NAF-1), connectin, adhesion molecules such as cell adhesion molecule(s) (CAM(s)) and the semaphorins/collapsins (Neugebauer et al., Neuron 6:345-58, 1991; O'Shea et al., Neuron 7:231-7, 1991; Osterhout et al., Devel. Biol. 150:256-65, 1992; Goodman, Cell 78:353-6, 1993; DeFreitas et al., Neuron 15:333-43, 1995; Dodd and Schuchardy Cell 81:471-4, 1995; Keynes and Cook, Cell 83:161-9, 1995; Müller et al., Cur. Opin. Genet. and Devel. 6:469-74, 1996, Goodman, Annu. Rev. Neurosci. 19:341-77, 1996; WIPO Patent Application No: 97/29189 and Goodman et al., U.S. Pat. No. 5,639,856).

Semaphorins/collapsins are a family of related transmembrane and secreted molecules. Invertebrate, vertebrate and viral semaphorins are known (Kolodkin et al., Cell 75:1389-99, 1993; Luo et al., Cell 75:217-27, 1993; Ensser and Fleckenstein, J. Gen. Virol. 76:1063-7, 1995; Luo et al, Neuron 14:1131-40, 1995; Adams et al., Mech. Devel. 57:33-45, 1996; Hall et al., Proc. Natl. Acad. Sci. USA 93:11780-8, 1996; Roche et al., Oncogene 12:1289-97, 1996; Skeido et al., Proc. Natl. Acad. Sci. USA 93:4120-5, 1996; Xiang et al., Genomics 32:39-48, 1996; Eckhardt et al., Mol. Cell Neurosci. 9:409-19, 1997 and Zhou et al., Mol. Cell. Neurosci. 9:26-41, 1997).

The semaphorins generally comprise an N-terminal variable region of 30-60 amino acids that includes a secretory signal sequence, followed by a conserved region of about 500 amino acid residues called the semaphorin or sema domain. The extracellular semaphorin domain contains conserved cysteine residues, an N-linked glycosylation site and numerous blocks of amino acid residues which are conserved though-out the family. Classification into five subgroups within the semaphorin family has made based on the sequence of the region C-terminal to the semaphorin domain. Both soluble (lacking a transmembrane domain) and membrane-bound (having a transmembrane domain and localized to a membrane) semaphorins have been described. See, for example, Kolodkin et al., ibid.; Adams et al., ibid. and Goodman et al., U.S. Pat. No. 5,639,856.

Group I semaphorins include semaphorins having a transmembrane domain followed by a cytoplasmic domain. Most insect semaphorins are membrane bound proteins and belong to Group I. G-Sema I, T-Sema I and D-Sema I have a region of 80 amino acid residues following the semaphorin domain, which is followed by a transmembrane domain and an 80-110 amino acid cytoplasmic domain. Murine Sema IVa has a transmembrane domain followed by a 216 amino acid cytoplasmic domain.

Groups II and III have no transmembrane domain or membrane association, but have a region with Ig homology. Group II secreted proteins, such as D-sema II, have a region of less than 20 amino acids between the semaphorin domain and an Ig-like domain followed by a short region of amino acid residues. Also included is alcelaphine herpesvirus type 1 semaphorin-like gene (avh-sema, Ensser and Fleckenstein, J. Gen. Virol. 76:1063-7, 1995) which ends with an Ig-like domain. Group III proteins, such as H-Sema III, are similar to Group II with the exception that the C-terminal amino acid region following the Ig-like domain is longer.

Group IV has a region of Ig homology C-terminal of the semaphorin domain followed by a transmembrane and cytoplasmic domain and includes semaphorins such as Sem B.

Group V has a series of thrombospondin repeats C-terminal of the semaphorin domain followed by a transmembrane and cytoplasmic domain and include murine sema F and G.

Other viral semaphorins such as vaccinia virus sema IV and variola virus sema IV, have a truncated, 441 amino acid residue, semaphorin domain and no Ig region. See Kolodkin et al., ibid.; Adams et al. ibid. and Zhou et al. ibid.

Overall semaphorins share the greatest degree of homology within the semaphorin domain and a greater degree of divergence in all other regions and domains, suggesting distinct roles for various sub-groups within the semaphorin family. The viral semaphorins are the most diverse.

Neurite growth cues are of great therapeutic value. Isolating and characterizing novel semaphorins would be of value for example, in modulating neurite growth and development; treatment of peripheral neuropathies; for use as therapeutics for the regeneration of neurons following strokes, brain damage caused by head injuries and paralysis caused by spinal injuries; diagnosing neurological diseases and in treating neurodegenerative diseases such as multiple sclerosis, Alzheimer's disease and Parkinson's disease. In addition, semaphorins are also being found in non-neuronal tissues and their usefulness for modulating cellular activation, homing, targeting, adhesion, proliferation and differentiation as well as mediating immunological responses is now being reported. The present invention addresses these needs and others by providing novel semaphorins and related compositions and methods.

### BRIEF DESCRIPTION OF THE DRAWING

The Figure is a hydrophobicity plot of ZSMF-16 using a Hopp/Woods hydrophilicity profile based on a sliding six-residue window, with buried G, S, and T residues and exposed H, Y, and W residues ignored.

### DESCRIPTION OF THE INVENTION

The present invention provides novel semaphorin polynucleotides, polypeptides and related compositions and methods.

Within one aspect, the present invention provides an isolated polynucleotide that encodes a semaphorin polypeptide comprising a sequence of amino acid residues that is at least 90% identical to an amino acid sequence selected from the group consisting of: (a) the amino acid sequence as shown in SEQ ID NO:2 from amino acid number 23 (Gly), to amino acid number 500 (Arg); (b) the amino acid sequence as shown in SEQ ID NO:2 from amino acid number 76 (Leu), to amino acid number 500 (Arg); (c) the amino acid sequence as shown in SEQ ID NO:2 from amino acid number 76 (Leu), to amino acid number 592 (Glu);); (d) the amino acid sequence as shown in SEQ ID NO:2 from amino acid number 76 (Leu), to amino acid number 654 (Thr); (e) the amino acid sequence as shown in SEQ ID NO:2 from amino acid number 76 (Leu), to amino acid number 779 (Thr); (f) the amino acid sequence as shown in SEQ ID NO:2 from amino acid number 23 (Gly), to amino acid number 779 (Thr); and (g) the amino acid sequence as shown in SEQ ID NO:2 from amino acid number 1 (Met), to amino acid number 779 (Thr). In one embodiment, the isolated polynucleotide disclosed above is selected from the group consisting of: (a) a polynucleotide sequence as shown in SEQ ID NO:1 from nucleotide 67 to nucleotide 1500; (b) a polynucleotide sequence as shown in SEQ ID NO:1 from nucleotide 226 to nucleotide 1500; (c) a polynucleotide sequence as shown in SEQ ID NO:1 from nucleotide 226 to nucleotide 1776; and (d) a polynucleotide sequence as shown in SEQ ID NO:1 from nucleotide 226 to nucleotide 1961; (e) a polynucleotide sequence as shown in SEQ ID NO:1 from nucleotide 226 to nucleotide 2337; (f) a polynucleotide sequence as shown in SEQ ID NO:1 from nucleotide 67 to nucleotide 2337; and (g) a polynucleotide sequence as shown in SEQ ID NO:1 from nucleotide 1 to nucleotide 2337. In another embodiment, the isolated polynucleotide disclosed above comprises nucleotide 1 to nucleotide 2337 of SEQ ID NO:3. In another embodiment, the isolated polynucleotide disclosed above encodes a polypeptide that comprises a sequence of amino acid residues selected from the group consisting of: (a) the amino acid sequence as shown in SEQ ID NO:2 from amino acid number 23 (Gly), to amino acid number 500 (Arg); (b) the amino acid sequence as shown in SEQ ID NO:2 from amino acid number 76 (Leu), to amino acid number 500 (Arg); (c) the amino acid sequence as shown in SEQ ID NO:2 from amino acid number 76 (Leu), to amino acid number 592 (Glu););(d) the amino acid sequence as shown in SEQ ID NO:2 from amino acid number 76 (Leu), to amino acid number 654 (Thr); (e) the amino acid sequence as shown in SEQ ID NO:2 from amino acid number 76 (Leu), to amino acid number 779 (Thr); (f) the amino acid sequence as shown in SEQ ID NO:2 from amino acid number 23 (Gly), to amino acid number 779 (Thr); and (g) the amino acid sequence as shown in SEQ ID NO:2 from amino acid number 1 (Met), to amino acid number 779 (Thr).

Within a second aspect, the present invention provides an expression vector comprising the following operably linked elements: a transcription promoter; a DNA segment encoding a semaphorin polypeptide as shown in SEQ ID NO:2 from amino acid number 23 (Gly), to amino acid number 779 (Thr); and a transcription terminator, wherein the promoter is operably linked to the DNA segment, and the DNA segment is operably linked to the transcription terminator. In one embodiment, the expression vector disclosed above, further comprising a secretory signal sequence operably linked to the DNA segment.

Within a third aspect, the present invention provides a cultured cell comprising an expression vector according as disclosed above, wherein the cell expresses a polypeptide encoded by the DNA segment.

Within another aspect, the present invention provides a DNA construct encoding a fusion protein, the DNA construct comprising: a first DNA segment encoding a polypeptide comprising a sequence of amino acid residues selected from the group consisting of: (a) the amino acid sequence as shown in SEQ ID NO:2 from amino acid number 1 (Met), to amino acid number 22 (Ser); (b) the amino acid sequence as shown in SEQ ID NO:2 from amino acid number 23 (Gly), to amino acid number 75 (Asn); (c) the amino acid sequence as shown in SEQ ID NO:2 from amino acid number 76 (Leu), to amino acid number 500 (Arg); (d) the amino acid sequence as shown in SEQ ID NO:2 from amino acid number 501 (Gln), to amino acid number 592 (Glu); (e) the amino acid sequence as shown in SEQ ID NO:2 from amino acid number 593 (His), to amino acid number 654 (Thr); (f) the amino acid sequence as shown in SEQ ID NO:2 from amino acid number 655 (Leu), to amino acid number 779 (Thr); (g) the amino acid sequence as shown in SEQ ID NO:2 from amino acid number 23 (Gly), to amino acid number 779 (Thr); and at least one other DNA segment encoding an additional polypeptide, wherein the first and other DNA segments are connected in-frame; and wherein the first and other DNA segments encode the fusion protein.

Within another aspect, the present invention provides an expression vector comprising the following operably linked elements: a transcription promoter; a DNA construct encoding a fusion protein as disclosed above; and a transcription terminator, wherein the promoter is operably linked to the DNA construct, and the DNA construct is operably linked to the transcription terminator.

Within another aspect, the present invention provides a cultured cell comprising an expression vector as disclosed above, wherein the cell expresses a polypeptide encoded by the DNA construct.

Within another aspect, the present invention provides a method of producing a fusion protein comprising: culturing a cell as disclosed above; and isolating the polypeptide produced by the cell.

Within another aspect, the present invention provides an isolated semaphorin polypeptide comprising a sequence of amino acid residues that is at least 90% identical to an amino acid sequence selected from the group consisting of: (a) the amino acid sequence as shown in SEQ ID NO:2 from amino acid number 23 (Gly), to amino acid number 500 (Arg); (b) the amino acid sequence as shown in SEQ ID NO:2 from amino acid number 76 (Leu), to amino acid number 500 (Arg); (c) the amino acid sequence as shown in SEQ ID NO:2 from amino acid number 76 (Leu), to amino acid number 592 (Glu);); (d) the amino acid sequence as shown in SEQ ID NO:2 from amino acid number 76 (Leu), to amino acid number 654 (Thr); (e) the amino acid sequence as shown in SEQ ID NO:2 from amino acid number 76 (Leu), to amino acid number 779 (Thr); (f) the amino acid sequence as shown in SEQ ID NO:2 from amino acid number 23 (Gly), to amino acid number 779 (Thr); and (g) the amino acid sequence as shown in SEQ ID NO:2 from amino acid number 1 (Met), to amino acid number 779 (Thr). In one embodiment, the isolated polypeptide disclosed above comprises a sequence of amino acid residues selected from the group consisting of: (a) the amino acid sequence as shown in SEQ ID NO:2 from amino acid number 23 (Gly), to amino acid number 500 (Arg); (b) the amino acid sequence as shown in SEQ ID NO:2 from amino acid number 76 (Leu), to amino acid number 500 (Arg); (c) the amino acid sequence as shown in SEQ ID NO:2 from amino acid number 76 (Leu), to amino acid number 592 (Glu);); (d) the amino acid sequence as shown in SEQ ID NO:2 from amino acid number 76 (Leu), to amino acid number 654 (Thr); (e) the amino acid sequence as shown in SEQ ID NO:2 from amino acid number 76 (Leu), to amino acid number 779 (Thr); (f) the amino acid sequence as shown in SEQ ID NO:2 from amino acid number 23 (Gly), to amino acid number 779 (Thr); and (g) the amino acid sequence as shown in SEQ ID NO:2 from amino acid number 1 (Met), to amino acid number 779 (Thr).

Within another aspect, the present invention provides a method of producing a semaphorin polypeptide comprising: culturing a cell as disclosed above; and isolating the semaphorin polypeptide produced by the cell.

Within another aspect, the present invention provides a method of producing an antibody comprising: inoculating an animal with a polypeptide selected from the group consisting of: (a) a polypeptide consisting of 9 to 19 amino acids, wherein the polypeptide is identical to a contiguous sequence of amino acids in SEQ ID NO:2 from amino acid number 39 (Gly) to amino acid number 57 (Tyr); (b) a polypeptide as disclosed above; (c) a polypeptide consisting of the amino acid sequence of SEQ ID NO:2 from amino acid number 39 (Gly) to 57 (Tyr); (d) a polypeptide consisting of the amino acid sequence of SEQ ID NO:2 from amino acid number 107 (Asp) to 114 (Ala) of SEQ ID NO:2; and wherein the polypeptide elicits an immune response in the animal to produce the antibody; and isolating the antibody from the animal.

Within another aspect, the present invention provides an antibody produced by the method as disclosed above, which specifically binds to a polypeptide of SEQ ID NO:2. In one embodiment the antibody disclosed above is a monoclonal antibody. Within another aspect, the present invention provides an antibody that specifically binds to a polypeptide as disclosed above.

Within another aspect, the present invention provides a method of detecting, in a test sample, the presence of a modulator of ZSMF-16 protein activity, comprising: transfecting a ZSMF-16-responsive cell, with a reporter gene construct that is responsive to a ZSMF-16-stimulated cellular pathway; and producing a ZSMF-16 polypeptide by the method as disclosed above; and adding the ZSMF-16 polypeptide to the cell, in the presence and absence of a test sample; and comparing levels of response to the ZSMF-16 polypeptide, in the presence and absence of the test sample, by a biological or biochemical assay; and determining from the comparison, the presence of the modulator of ZSMF-16 activity in the test sample.

Within another aspect, the present invention provides a method for detecting a genetic abnormality in a patient, comprising: obtaining a genetic sample from a patient; producing a first reaction product by incubating the genetic sample with a polynucleotide comprising at least 14 contiguous nucleotides of SEQ ID NO:1 or the complement of SEQ ID NO:1, under conditions wherein said polynucleotide will hybridize to complementary polynucleotide sequence; visualizing the first reaction product; and comparing said first reaction product to a control reaction product from a wild type patient, wherein a difference between said first reaction product and said control reaction product is indicative of a genetic abnormality in the patient.

Within another aspect, the present invention provides a method for detecting a cancer in a patient, comprising: obtaining a tissue or biological sample from a patient; incubating the tissue or biological sample with an antibody that specifically binds SEQ ID NO:2 under conditions wherein the antibody binds to its complementary polypeptide in the tissue or biological sample; visualizing the antibody bound in the tissue or biological sample; and comparing levels of antibody bound in the tissue or biological sample from the patient to a normal control tissue or biological sample, wherein an increase or decrease in the level of antibody bound to the patient tissue or biological sample relative to the normal control tissue or biological sample is indicative of a cancer in the patient.

Within another aspect, the present invention provides a method for detecting a cancer in a patient, comprising: obtaining a tissue or biological sample from a patient; labeling a polynucleotide comprising at least 14 contiguous nucleotides of SEQ ID NO:1 or the complement of SEQ ID NO:1; incubating the tissue or biological sample with under conditions wherein the polynucleotide will hybridize to complementary polynucleotide sequence; visualizing the labeled polynucleotide in the tissue or biological sample; and comparing the level of labeled polynucleotide hybridization in the tissue or biological sample from the patient to a normal control tissue or biological sample, wherein an increase or decrease in the labeled polynucleotide hybridization to the patient tissue or biological sample relative to the normal control tissue or biological sample is indicative of a cancer in the patient.

These and other aspects of the invention will become evident upon reference to the following detailed description and the attached drawing.

Prior to setting forth the invention, it may be helpful to an understanding thereof to set forth definitions of certain terms to be used hereinafter:

The term “affinity tag” is used herein to denote a polypeptide segment that can be attached to a second polypeptide to provide for purification of the second polypeptide or provide sites for attachment of the second polypeptide to a substrate. In principal, any peptide or protein for which an antibody or other specific binding agent is available can be used as an affinity tag. Affinity tags include a poly-histidine tract, protein A (Nilsson et al., EMBO J. 4:1075, 1985; Nilsson et al., Methods Enzymol. 198:3, 1991), glutathione S transferase (Smith and Johnson, Gene 67:31, 1988), Glu—Glu affinity tag (Grussenmeyer et al., Proc. Natl. Acad. Sci. USA 82:7952-4, 1995), substance P, Flag™ peptide (Hopp et al., Biotechnology 6:1204-10, 1988), streptavidin binding peptide, or other antigenic epitope or binding domain. See, in general, Ford et al., Protein Expression and Purification 2: 95-107, 1991. DNAs encoding affinity tags are available from commercial suppliers (e.g., Pharmacia Biotech, Piscataway, N.J.).

The term “allelic variant” is used herein to denote any of two or more alternative forms of a gene occupying the same chromosomal locus. Allelic variation arises naturally through mutation, and may result in phenotypic polymorphism within populations. Gene mutations can be silent (no change in the encoded polypeptide) or may encode polypeptides having altered amino acid sequence. The term allelic variant is also used herein to denote a protein encoded by an allelic variant of a gene.

The terms “amino-terminal” and “carboxyl-terminal” are used herein to denote positions within polypeptides. Where the context allows, these terms are used with reference to a particular sequence or portion of a polypeptide to denote proximity or relative position. For example, a certain sequence positioned carboxyl-terminal to a reference sequence within a polypeptide is located proximal to the carboxyl terminus of the reference sequence, but is not necessarily at the carboxyl terminus of the complete polypeptide.

The term “complements of a polynucleotide molecule” is a polynucleotide molecule having a complementary base sequence and reverse orientation as compared to a reference sequence. For example, the sequence 5′ ATGCACGGG 3′ is complementary to 5′ CCCGTGCAT 3′.

The term “contig” denotes a polynucleotide that has a contiguous stretch of identical or complementary sequence to another polynucleotide. Contiguous sequences are said to “overlap” a given stretch of polynucleotide sequence either in their entirety or along a partial stretch of the polynucleotide. For example, representative contigs to the polynucleotide sequence 5′-ATGGAGCTT-3′ are 5′-AGCTTgagt-3′ and 3′-tcgacTACC-5′.

The term “degenerate nucleotide sequence” denotes a sequence of nucleotides that includes one or more degenerate codons (as compared to a reference polynucleotide molecule that encodes a polypeptide). Degenerate codons contain different triplets of nucleotides, but encode the same amino acid residue (i.e., GAU and GAC triplets each encode Asp).

The term “expression vector” is used to denote a DNA molecule, linear or circular, that comprises a segment encoding a polypeptide of interest operably linked to additional segments that provide for its transcription. Such additional segments include promoter and terminator sequences, and may also include one or more origins of replication, one or more selectable markers, an enhancer, a polyadenylation signal, etc. Expression vectors are generally derived from plasmid or viral DNA, or may contain elements of both.

The term “isolated”, when applied to a polynucleotide, denotes that the polynucleotide has been removed from its natural genetic milieu and is thus free of other extraneous or unwanted coding sequences, and is in a form suitable for use within genetically engineered protein production systems. Such isolated molecules are those that are separated from their natural environment and include cDNA and genomic clones. Isolated DNA molecules of the present invention are free of other genes with which they are ordinarily associated, but may include naturally occurring 5′ and 3′ untranslated regions such as promoters and terminators. The identification of associated regions will be evident to one of ordinary skill in the art (see for example, Dynan and Tijan, Nature 316:774-78, 1985).

An “isolated” polypeptide or protein is a polypeptide or protein that is found in a condition other than its native environment, such as apart from blood and animal tissue. In a preferred form, the isolated polypeptide is substantially free of other polypeptides, particularly other polypeptides of animal origin. It is preferred to provide the polypeptides in a highly purified form, i.e. greater than 95% pure, more preferably greater than 99% pure. When used in this context, the term “isolated” does not exclude the presence of the same polypeptide in alternative physical forms, such as dimers or alternatively glycosylated or derivatized forms.

The term “operably linked”, when referring to DNA segments, indicates that the segments are arranged so that they function in concert for their intended purposes, e.g., transcription initiates in the promoter and proceeds through the coding segment to the terminator.

The term “ortholog” denotes a polypeptide or protein obtained from one species that is the functional counterpart of a polypeptide or protein from a different species. Sequence differences among orthologs are the result of speciation.

“Paralogs” are distinct but structurally related proteins made by an organism. Paralogs are believed to arise through gene duplication. For example, α-globin, β-globin, and myoglobin are paralogs of each other.

A “polynucleotide” is a single- or double-stranded polymer of deoxyribonucleotide or ribonucleotide bases read from the 5′ to the 3′ end. Polynucleotides include RNA and DNA, and may be isolated from natural sources, synthesized in vitro, or prepared from a combination of natural and synthetic molecules. Sizes of polynucleotides are expressed as base pairs (abbreviated “bp”), nucleotides (“nt”), or kilobases (“kb”). Where the context allows, the latter two terms may describe polynucleotides that are single-stranded or double-stranded. When the term is applied to double-stranded molecules it is used to denote overall length and will be understood to be equivalent to the term “base pairs”. It will be recognized by those skilled in the art that the two strands of a double-stranded polynucleotide may differ slightly in length and that the ends thereof may be staggered as a result of enzymatic cleavage; thus all nucleotides within a double-stranded polynucleotide molecule may not be paired.

A “polypeptide” is a polymer of amino acid residues joined by peptide bonds, whether produced naturally or synthetically. Polypeptides of less than about 10 amino acid residues are commonly referred to as “peptides”.

“Probes and/or primers” as used herein can be RNA or DNA. DNA can be either cDNA or genomic DNA. Polynucleotide probes and primers are single or double-stranded DNA or RNA, generally synthetic oligonucleotides, but may be generated from cloned cDNA or genomic sequences or its complements. Analytical probes will generally be at least 20 nucleotides in length, although somewhat shorter probes (14-17 nucleotides) can be used. PCR primers are at least 5 nucleotides in length, preferably 15 or more nt, more preferably 20-30 nt. Short polynucleotides can be used when a small region of the gene is targeted for analysis. For gross analysis of genes, a polynucleotide probe may comprise an entire exon or more. Probes can be labeled to provide a detectable signal, such as with an enzyme, biotin, a radionuclide, fluorophore, chemiluminescer, paramagnetic particle and the like, which are commercially available from many sources, such as Molecular Probes, Inc., Eugene, Oreg., and Amersham Corp., Arlington Heights, Ill., using techniques that are well known in the art.

The term “promoter” is used herein for its art-recognized meaning to denote a portion of a gene containing DNA sequences that provide for the binding of RNA polymerase and initiation of transcription. Promoter sequences are commonly, but not always, found in the 5′ non-coding regions of genes.

A “protein” is a macromolecule comprising one or more polypeptide chains. A protein may also comprise non-peptidic components, such as carbohydrate groups. Carbohydrates and other non-peptidic substituents may be added to a protein by the cell in which the protein is produced, and will vary with the type of cell. Proteins are defined herein in terms of their amino acid backbone structures; substituents such as carbohydrate groups are generally not specified, but may be present nonetheless.

The term “receptor” denotes a cell-associated protein that binds to a bioactive molecule (i.e., a ligand) and mediates the effect of the ligand on the cell. Membrane-bound receptors are characterized by a multi-domain structure comprising an extracellular ligand-binding domain and an intracellular effector domain that is typically involved in signal transduction. Binding of ligand to receptor results in a conformational change in the receptor that causes an interaction between the effector domain and other molecule(s) in the cell. This interaction in turn leads to an alteration in the metabolism of the cell. Metabolic events that are linked to receptor-ligand interactions include gene transcription, phosphorylation, dephosphorylation, increases in cyclic AMP production, mobilization of cellular calcium, mobilization of membrane lipids, cell adhesion, hydrolysis of inositol lipids and hydrolysis of phospholipids. In general, receptors can be membrane bound, cytosolic or nuclear; monomeric (e.g., thyroid stimulating hormone receptor, beta-adrenergic receptor) or multimeric (e.g., PDGF receptor, growth hormone receptor, IL-3 receptor, GM-CSF receptor, G-CSF receptor, erythropoietin receptor and IL-6 receptor).

The term “secretory signal sequence” denotes a DNA sequence that encodes a polypeptide (a “secretory peptide”) that, as a component of a larger polypeptide, directs the larger polypeptide through a secretory pathway of a cell in which it is synthesized. The larger polypeptide is commonly cleaved to remove the secretory peptide during transit through the secretory pathway.

The term “splice variant” is used herein to denote alternative forms of RNA transcribed from a gene. Splice variation arises naturally through use of alternative splicing sites within a transcribed RNA molecule, or less commonly between separately transcribed RNA molecules, and may result in several mRNAs transcribed from the same gene. Splice variants may encode polypeptides having altered amino acid sequence. The term splice variant is also used herein to denote a protein encoded by a splice variant of an mRNA transcribed from a gene.

Molecular weights and lengths of polymers determined by imprecise analytical methods (e.g., gel electrophoresis) will be understood to be approximate values. When such a value is expressed as “about” X or “approximately” X, the stated value of X will be understood to be accurate to ±10%.

All references cited herein are incorporated by reference in their entirety.

The present invention is based in part upon the discovery of a novel human member of the semaphorin family, designated “ZSMF-16”. The human ZSMF-16 nucleotide sequence is represented in SEQ ID NO:1 and the deduced amino acid sequence in SEQ ID NO:2.

The novel human ZSMF-16 semaphorin proteins and polypeptides encoded by polynucleotides of the present invention are homologous to conserved motifs within the semaphorin family. Sequence analysis of the deduced amino acid sequence as represented in SEQ ID NO:2 indicates a 779 amino acid polypeptide containing a 22 amino acid residue secretory signal sequence (amino acid residues 1 (Met) to 22 (Ser) of SEQ ID NO:2), and a mature polypeptide of 757 amino acids (amino acid residues 23 (Gly) to 779 (Thr)). The mature zsmf-16 polypeptide sequence contains the following domains, and motifs:

(1) an “N-terminal region” comprising amino acid residues 23 (Gly) to 75 (Asn) of SEQ ID NO:2); followed by

(2) a 425 amino acid residue semaphorin domain (a.k.a., sema domain) comprising amino acid residues 76 (Leu) to 500 (Arg) of SEQ ID NO:2). Within the semaphorin domain there are 8 conserved cysteine residues comprising amino acid residues 113, 131, 140, 167, 267, 291, 339, and 379 of SEQ ID NO:2);

(3) the semaphorin domain is followed by “middle domain” of a stretch of amino acid residues comprising amino acid residues 501 (Gln) to 592 (Glu) of SEQ ID NO:2. There appears to be no strong transmembrane domain or membrane linkage. The middle domain is followed by

(4) an “Ig-like domain” comprising amino acid residues 593 (His) to 654 (Thr) of SEQ ID NO:2). Within the Ig-like domain are 2 conserved cysteines comprising amino acid residues 600 and 652 of SEQ ID NO:2. The Ig-like domain is followed by

(5) a “C-terminal domain” comprising amino acid residues 655 (Leu) to 738 (Phe) of SEQ ID NO:2. Within the C-terminal domain is a “basic domain” at the C-terminus of the ZSMF-16 polypeptide comprising approximately amino acid residue 739 (Arg) to 779 (Thr).

Moreover N-linked glycosylation sites at Asn residues comprising amino acid residues 62, 124, and 594. Moreover an ATP/GTP binding site motif is present from amino acid 744 (Gly) to 751 (Ser).

Moreover the genomic structure of ZSMF-16 is readily determined by one of skill in the art by comparing the cDNA sequence of SEQ ID NO:1 and the translated amino acid of SEQ ID NO:2 with the genomic DNA in which the gene is contained (Genbank Accession No. AC006208). For example, such analysis can be readily done using FASTA as described herein. As such, the intron and exon junctions in this region of genomic DNA can be determined for the ZSMF-16 gene. Thus, the present invention includes the ZSMF-16 gene as located in human genomic DNA.

Those skilled in the art will recognize that domain boundaries are approximations based on sequence alignments, intron positions and splice sites, and may vary slightly; however, such estimates are generally accurate to within ±4 amino acid residues.

The present invention is not limited to the expression of the sequence shown in SEQ ID NO:1. A number of truncated ZSMF-16 polynucleotides and polypeptides are provided by the present invention. These polypeptides can be produced by expressing polynucleotides encoding them in a variety of host cells. In many cases, the structure of the final polypeptide product will result from processing of the nascent polypeptide chain by the host cell, thus the final sequence of a ZSMF-16 polypeptide produced by a host cell will not always correspond to the full sequence encoded by the expressed polynucleotide. For example, expressing the complete ZSMF- 16 sequence in a cultured mammalian cell is expected to result in removal of at least the secretory peptide, while the same polypeptide produced in a prokaryotic host would not be expected to be cleaved. By selecting particular combinations of polynucleotide and host cell, a variety of ZSMF-16 polypeptides can thus be produced. Differential processing may result in heterogeneity of expressed polypeptides and the production of heterodimeric ZSMF-16 proteins. In addition, ZSMF-16 polypeptides can be produced by other known methods, such as solid phase synthesis, methods for which are well known in the art. See, for example, Merrifield, J. Am. Chem. Soc. 85:2149, 1963; Stewart et al., Solid Phase Peptide Synthesis (2nd edition), Pierce Chemical Co., Rockford, Ill., 1984; Bayer and Rapp, Chem. Pept. Prot. 3:3, 1986; and Atherton et al., Solid Phase Peptide Synthesis: A Practical Approach, IRL Press, Oxford, 1989.

Based on electronic Northern data, ZSMF-16 polynucleotides were expressed in mammary tumor tissue, breast tumor and diseased breast tissues, liver, small intestine, bone and brain tissue. Moreover, Northern blot analysis is expected to show that a transcript is detected corresponding to ZSMF-16 in other neuronal tissues, aside from brain, such as spinal cord, and perhaps non-neuronal tissues such as testis, spleen and placenta. The transcript size should agree with the predicted size of the ZSMF-16 protein as disclosed in SEQ ID NO:2. Additional analysis may reveal a ZSMF-16 transcript in numerous localized brain and neuronal tissues, and in tumor cell lines. RT-PCR data can also be performed to suggest where ZSMF-16 mRNA is expressed. Such methods are well known in the art and disclosed herein.

The present invention also provides polynucleotide molecules, including DNA and RNA molecules, that encode the ZSMF-16 polypeptides disclosed herein. Those skilled in the art will readily recognize that, in view of the degeneracy of the genetic code, considerable sequence variation is possible among these polynucleotide molecules. A degenerate polynucleotide sequence that encompasses all polynucleotides that encode the ZSMF-16 polypeptide of SEQ ID NO:2 (amino acid residues 1-779) is disclosed in SEQ ID NO:3. Thus, ZSMF-16 polypeptide-encoding polynucleotides ranging from nucleotide 1-2337 of SEQ ID NO:3 are contemplated by the present invention. Also contemplated by the present invention are fragments as described herein with respect to SEQ ID NO:1, which are formed from analogous regions of SEQ ID NO:3, wherein nucleotides 1-2337 of SEQ ID NO:1 correspond to nucleotides 1-2337 SEQ ID NO:3. Those skilled in the art will recognize that the degenerate sequence of SEQ ID NO:3 also provides all RNA sequences encoding SEQ ID NO:2 by substituting uracil (U) for thymine (T). The RNA equivalents of the herein named sequences are also contemplated by the present invention. Table 1 sets forth the one-letter nucleotide base codes used within SEQ ID NO:3 to denote degenerate nucleotide positions. “Resolutions” are the nucleotides denoted by a nucleotide base code letter. “Complement” indicates the nucleotide base code for the complementary nucleotide(s). For example, the nucleotide base code “Y” denotes either the nucleotide C or T, and its complement nucleotide base code “R” denotes nucleotides A or G, A being complementary to T, and G being complementary to C.

TABLE 1

| Nucleotide | | Nucleotide | |
|---|---|---|---|
| Base Code | Resolution | Base Code | Complement |
| A | A | T | T |
| C | C | G | G |
| G | G | C | C |
| T | T | A | A |
| R | A\|G | Y | C\|T |
| Y | C\|T | R | A\|G |
| M | A\|C | K | G\|T |
| K | G\|T | M | A\|C |
| S | C\|G | S | C\|G |
| W | A\|T | W | A\|T |
| H | A\|C\|T | D | A\|G\|T |
| B | C\|G\|T | V | A\|C\|G |
| V | A\|C\|G | B | C\|G\|T |
| D | A\|G\|T | H | A\|C\|T |
| N | A\|C\|G\|T | N | A\|C\|G\|T |

The degenerate codons used in SEQ ID NO:3, encompassing all possible codons for a given amino acid, are set forth in Table 2.

TABLE 2

| Three | One | | |
|---|---|---|---|
| Letter | Letter | | Degenerate |
| Code | Code | Synonymous Codons | Codon |
| Cys | C | TGC TGT | TGY |
| Ser | S | AGC AGT TCA TCC TCG TCT | WSN |
| Thr | T | ACA ACC ACG ACT | ACN |
| Pro | P | CCA CCC CCG CCT | CCN |
| Ala | A | GCA GCC GCG GCT | GCN |
| Gly | G | GGA GGC GGG GGT | GGN |
| Asn | N | AAC AAT | AAY |
| Asp | D | GAG GAT | GAY |
| Glu | E | GAA GAG | GAR |
| Gln | Q | CAA CAG | CAR |
| His | H | CAC CAT | CAY |
| Arg | R | AGA AGG CGA CGC CGG CGT | MGN |
| Lys | K | AAA AAG | AAR |
| Met | M | ATG | ATG |
| Ile | I | ATA ATC ATT | ATH |
| Leu | L | CTA CTC CTG CTT TTA TTG | YTN |
| Val | V | GTA GTC GTG GTT | GTN |
| Phe | F | TTC TTT | TTY |
| Tyr | Y | TAC TAT | TAY |
| Trp | W | TGG | TGG |
| Ter | . | TAA TAG TGA | TRR |
| Asn\|Asp | B | | RAY |
| Glu\|Gln | Z | | SAR |
| Any | X | | NNN |

One of ordinary skill in the art will appreciate that some ambiguity is introduced in determining a degenerate codon, representative of all possible codons encoding each amino acid. For example, the degenerate codon for serine (WSN) can, in some circumstances, encode arginine (AGR), and the degenerate codon for arginine (MGN) can, in some circumstances, encode serine (AGY). A similar relationship exists between codons encoding phenylalanine and leucine. Thus, some polynucleotides encompassed by the degenerate sequence may encode variant amino acid sequences, but one of ordinary skill in the art can easily identify such variant sequences by reference to the amino acid sequence of SEQ ID NO:2. Such variant sequences can be readily tested for functionality as disclosed herein.

One of ordinary skill in the art will also appreciate that different species can exhibit “preferential codon usage.” In general, see, Grantham, et al., Nuc. Acids Res., 8:1893-912, 1980; Haas, et al. Curr. Biol., 6:315-24, 1996; Wain-Hobson, et al., Gene, 13:355-64, 1981; Grosjean and Fiers, Gene, 18:199-209, 1982; Holm, Nuc. Acids Res. 14:3075-87, 1986; Ikemura, J. Mol. Biol., 158:573-97, 1982. As used herein, the term “preferential codon usage” or “preferential codons” is a term of art referring to protein translation codons that are most frequently used in cells of a certain species, thus favoring one or a few representatives of all of the possible codons encoding each amino acid (See Table 2). For example, the amino acid threonine (Thr) may be encoded by ACA, ACC, ACG, or ACT, but in mammalian cells ACC is the most commonly used codon; in other species, for example, insect, yeast, viruses or bacteria, different Thr codons may be preferential. Preferential codons for a particular species can be introduced into the polynucleotides of the present invention by a variety of methods known in the art. Introduction of preferential codon sequences into recombinant DNA can, for example, enhance production of the protein by making protein translation more efficient within a particular cell type or species. Therefore, the degenerate codon sequence disclosed in SEQ ID NO:3 serves as a template for optimizing expression of polynucleotides in various cell types and species commonly used in the art and disclosed herein. Sequences containing preferential codons can be tested and optimized for expression in various species, and tested for functionality as disclosed herein.

The highly conserved amino acids in the semaphorin domain of ZSMF-16 can be used as a tool to identify new family members. For instance, reverse transcription-polymerase chain reaction (RT-PCR) can be used to amplify sequences, in particular, those sequences encoding the conserved semaphorin domain, especially sequences associated with the conserved cysteine residues, from RNA obtained from a variety of tissue sources or cell lines. In particular, highly degenerate primers designed from the ZSMF-16 nucleotide sequences as disclosed in SEQ ID NO:1 and SEQ ID NO:3 are useful for this purpose.

Within preferred embodiments of the invention, isolated polynucleotides will hybridize to similar sized regions of SEQ ID NO:1, or to sequences complementary thereto, under stringent conditions. Within preferred embodiments of the invention the isolated polynucleotides will hybridize to similar sized regions of SEQ ID NO:1, other polynucleotide probes, primers, fragments and sequences recited herein or sequences complementary thereto. Polynucleotide hybridization is well known in the art and widely used for many applications, see for example, Sambrook et al., Molecular Cloning: A Laboratory Manual, Second Edition, Cold Spring Harbor, N.Y., 1989; Ausubel et al., eds., Current Protocols in Molecular Biology, John Wiley and Sons, Inc., N.Y., 1987; Berger and Kimmel, eds., Guide to Molecular Cloning Techniques, Methods in Enzymology, volume 152, 1987 and Wetmur, Crit. Rev. Biochem. Mol. Biol. 26:227-59, 1990. Polynucleotide hybridization exploits the ability of single stranded complementary sequences to form a double helix hybrid. Such hybrids include DNA-DNA, RNA-RNA and DNA-RNA.

Hybridization will occur between sequences which contain some degree of complementarity. Hybrids can tolerate mismatched base pairs in the double helix, but the stability of the hybrid is influenced by the degree of mismatch. The Tₘ of the mismatched hybrid decreases by 1° C. for every 1-1.5% base pair mismatch. Varying the stringency of the hybridization conditions allows control over the degree of mismatch that will be present in the hybrid. The degree of stringency increases as the hybridization temperature increases and the ionic strength of the hybridization buffer decreases. Stringent hybridization conditions encompass temperatures of about 5-25° C. below the thermal melting point (Tₘ) of the hybrid and a hybridization buffer having up to 1 M Na⁺. Higher degrees of stringency at lower temperatures can be achieved with the addition of formamide which reduces the Tₘ of the hybrid about 1° C. for each 1% formamide in the buffer solution. Generally, such stringent conditions encompass temperatures of 20-70° C. and a hybridization buffer containing up to 6X SSC and 0-50% formamide. A higher degree of stringency can be achieved at temperatures of from 40-70° C. with a hybridization buffer having up to 4X SSC and from 0-50% formamide. Highly stringent conditions typically encompass temperatures of 42-70° C. with a hybridization buffer having up to 1X SSC and 0-50% formamide. Different degrees of stringency can be used during hybridization and washing to achieve maximum specific binding to the target sequence. Typically, the washes following hybridization are performed at increasing degrees of stringency to remove non-hybridized polynucleotide probes from hybridized complexes.

The above conditions are meant to serve as a guide and it is well within the abilities of one skilled in the art to adapt these conditions for use with a particular polypeptide hybrid. The Tₘ for a specific target sequence is the temperature (under defined conditions) at which 50% of the target sequence will hybridize to a perfectly matched probe sequence. Those conditions which influence the Tₘ include, the size and base pair content of the polynucleotide probe, the ionic strength of the hybridization solution, and the presence of destabilizing agents in the hybridization solution. Numerous equations for calculating Tₘ are known in the art, see for example (Sambrook et al., ibid.; Ausubel et al., ibid.; Berger and Kimmel, ibid. and Wetmur, ibid.) and are specific for DNA, RNA and DNA-RNA hybrids and polynucleotide probe sequences of varying length. Sequence analysis software such as Oligo 4.0 (publicly available shareware) and Primer Premier (PREMIER Biosoft International, Palo Alto, Calif.) as well as sites on the Internet, are available tools for analyzing a given sequence and calculating Tₘ based on user defined criteria. Such programs can also analyze a given sequence under defined conditions and suggest suitable probe sequences. Typically, hybridization of longer polynucleotide sequences, >50 bp, is done at temperatures of about 20-25° C. below the calculated Tₘ. For smaller probes, <50 bp, hybridization is typically carried out at the Tₘ or 5-10° C. below. This allows for the maximum rate of hybridization for DNA-DNA and DNA-RNA hybrids.

The length of the polynucleotide sequence influences the rate and stability of hybrid formation. Smaller probe sequences, <50 bp, come to equilibrium with complementary sequences rapidly, but may form less stable hybrids. Incubation times of anywhere from minutes to hours can be used to achieve hybrid formation. Longer probe sequences come to equilibrium more slowly, but form more stable complexes even at lower temperatures. Incubations are allowed to proceed overnight or longer. Generally, incubations are carried out for a period equal to three times the calculated Cot time. Cot time, the time it takes for the polynucleotide sequences to reassociate, can be calculated for a particular sequence by methods known in the art.

The base pair composition of polynucleotide sequence will effect the thermal stability of the hybrid complex, thereby influencing the choice of hybridization temperature and the ionic strength of the hybridization buffer. A-T pairs are less stable than G-C pairs in aqueous solutions containing NaCl. Therefore, the higher the G-C content, the more stable the hybrid. Even distribution of G and C residues within the sequence also contribute positively to hybrid stability. Base pair composition can be manipulated to alter the Tₘ of a given sequence, for example, 5-methyldeoxycytidine can be substituted for deoxycytidine and 5-bromodeoxuridine can be substituted for thymidine to increase the Tₘ. 7-deazo-2′-deoxyguanosine can be substituted for guanosine to reduce dependence on Tₘ.

Ionic concentration of the hybridization buffer also effects the stability of the hybrid. Hybridization buffers generally contain blocking agents such as Denhardt's solution (Sigma Chemical Co., St. Louis, Mo.), denatured salmon sperm DNA, tRNA, milk powders (BLOTTO), heparin or SDS, and a Na⁺ source, such as SSC (1X SSC: 0.15 M NaCl, 15 mM sodium citrate) or SSPE (1X SSPE: 1.8 M NaCl, 10 mM NaH₂PO₄, 1 mM EDTA, pH 7.7). By decreasing the ionic concentration of the buffer, the stability of the hybrid is increased. Typically, hybridization buffers contain from between 10 mM-1 M Na⁺. Premixed hybridization solutions are also available from commercial sources such as Clontech Laboratories (Palo Alto, Calif.) and Promega Corporation (Madison, Wis.) for use according to manufacturer's instruction. Addition of destabilizing or denaturing agents such as formamide, tetralkylammonium salts, guanidinium cations or thiocyanate cations to the hybridization solution will alter the Tₘ of a hybrid. Typically, formamide is used at a concentration of up to 50% to allow incubations to be carried out at more convenient and lower temperatures. Formamide also acts to reduce non-specific background when using RNA probes.

As previously noted, the isolated polynucleotides of the present invention include DNA and RNA. Methods for isolating DNA and RNA are well known in the art. In general, RNA is isolated from a tissue or cell that produces large amounts of ZSMF-16 RNA. Such tissues and cells are identified by Northern blotting (Thomas, Proc. Natl. Acad. Sci. USA 77:5201, 1980), and include breast, brain and neuronal tissues, although DNA can also be prepared using RNA from other tissues or isolated as genomic DNA. Total RNA can be prepared using guanidine isothiocyanate extraction followed by isolation by centrifugation in a CsCl gradient (Chirgwin et al., Biochemistry 18:52-94, 1979). Poly (A)⁺ RNA is prepared from total RNA using the method of Aviv and Leder (Proc. Natl. Acad. Sci. USA 69:1408-12, 1972). Complementary DNA (cDNA) is prepared from poly(A)⁺ RNA using known methods. Polynucleotides encoding ZSMF-16 polypeptides are then identified and isolated by, for example, hybridization or PCR.

A full-length clone encoding ZSMF-16 can be obtained by conventional cloning procedures. Complementary DNA (cDNA) clones are preferred, although for some applications (e.g., expression in transgenic animals) it may be preferable to use a genomic clone, or to modify a cDNA clone to include at least one genomic intron. Methods for preparing cDNA and genomic clones are well known and within the level of ordinary skill in the art, and include the use of the sequence disclosed herein, or parts thereof, for probing or priming a library. Expression libraries can be probed with antibodies to ZSMF-16, receptor fragments, or other specific binding partners.

The polynucleotides of the present invention can also be synthesized using techniques widely known in the art. See, for example, Glick and Pasternak, Molecular Biotechnology, Principles & Applications of Recombinant DNA, (ASM Press, Washington, D.C. 1994); Itakura et al., Annu. Rev. Biochem. 53: 323-56, 1984 and Climie et al., Proc. Natl. Acad. Sci. USA 87:633-7, 1990.

ZSMF-16 polynucleotide sequences disclosed herein can also be used as probes or primers to clone 5′ non-coding regions of a ZSMF-16 gene. In view of tissue-specific expression for ZSMF-16 elucidated by Northern blotting, this gene region is expected to provide for neurological, endrocrinological or tumor-specific expression. Promoter elements from a ZSMF-16 gene could thus be used to direct the tissue-specific expression of heterologous genes in, for example, transgenic animals or patients treated with gene therapy. Cloning of 5′ flanking sequences also facilitates production of ZSMF-16 proteins by “gene activation” as disclosed in U.S. Pat. No. 5,641,670. Briefly, expression of an endogenous ZSMF-16 gene in a cell is altered by introducing into the ZSMF-16 locus a DNA construct comprising at least a targeting sequence, a regulatory sequence, an exon, and an unpaired splice donor site. The targeting sequence is a ZSMF-16 5′ non-coding sequence that permits homologous recombination of the construct with the endogenous ZSMF-16 locus, whereby the sequences within the construct become operably linked with the endogenous ZSMF-16 coding sequence. In this way, an endogenous ZSMF-16 promoter can be replaced or supplemented with other regulatory sequences to provide enhanced, tissue-specific, or otherwise regulated expression.

The present invention further provides counterpart ligands and polynucleotides from other species (orthologs). These species include, but are not limited to, mammalian, avian, amphibian, reptile, fish, insect and other vertebrate and invertebrate species. Of particular interest are ZSMF-16 polypeptides from other mammalian species, including murine, porcine, ovine, bovine, canine, feline, equine, and other primate polypeptides. Orthologs of human ZSMF-16 can be cloned using information and compositions provided by the present invention in combination with conventional cloning techniques. For example, a cDNA can be cloned using mRNA obtained from a tissue or cell type that expresses the ligand. Suitable sources of mRNA can be identified by probing Northern blots with probes designed from the sequences disclosed herein. A library is then prepared from mRNA of a positive tissue or cell line. A ligand-encoding cDNA can then be isolated by a variety of methods, such as by probing with a complete or partial human cDNA or with one or more sets of degenerate probes based on the disclosed sequence. A cDNA can also be cloned by PCR, using primers designed from the sequences disclosed herein. Within an additional method, the cDNA library can be used to transform or transfect host cells, and expression of the cDNA of interest can be detected with an antibody to the ligand. Similar techniques can also be applied to the isolation of genomic clones.

Those skilled in the art will recognize that the sequence disclosed in SEQ ID NO:1 represents a single allele of the human ZSMF-16 gene and that allelic variation and alternative splicing are expected to occur. Allelic variants of this sequence can be cloned by probing cDNA or genomic libraries from different individuals according to standard procedures. Allelic variants of the DNA sequence shown in SEQ ID NO:2, including those containing silent mutations and those in which mutations result in amino acid sequence changes, are within the scope of the present invention, as are proteins which are allelic variants of SEQ ID NO:2. cDNAs generated from alternatively spliced mRNAs, which retain the properties of the ZSMF-16 polypeptide are included within the scope of the present invention, as are polypeptides encoded by such cDNAs and mRNAs. Allelic variants and splice variants of these sequences can be cloned by probing cDNA or genomic libraries from different individuals or tissues according to standard procedures known in the art.

The present invention also provides isolated ZSMF-16 polypeptides that are substantially similar to the polypeptides of SEQ ID NO:2 and their orthologs. The term “substantially similar” is used herein to denote polypeptides having 50%, preferably 60%, more preferably at least 80%, sequence identity to the sequences shown in SEQ ID NO:2 or their orthologs. Such polypeptides will more preferably be at least 90% identical, and most preferably 95% or more identical to SEQ ID NO:2 or its orthologs). Percent sequence identity is determined by conventional methods. See, for example, Altschul et al., Bull. Math. Bio. 48: 603-16, 1986 and Henikoff and Henikoff, Proc. Natl. Acad. Sci. USA 89:10915-9, 1992. Briefly, two amino acid sequences are aligned to optimize the alignment scores using a gap opening penalty of 10, a gap extension penalty of 1, and the “blosum 62” scoring matrix of Henikoff and Henikoff (ibid.) as shown in Table 3 (amino acids are indicated by the standard one-letter codes). The percent identity is then calculated as:

$$\frac{\text{Total number of identical matches}}{\text{[length of the longer sequence plus the number of gaps introduced into the longer sequence in order to align the two sequences]}} \times 100$$

TABLE 3

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | | | | | | | | | | | | | | | | | | | |
| R | −1 | 5 | | | | | | | | | | | | | | | | | | |
| N | −2 | 0 | 6 | | | | | | | | | | | | | | | | | |
| D | −2 | −2 | 1 | 6 | | | | | | | | | | | | | | | | |
| C | 0 | −3 | −3 | −3 | 9 | | | | | | | | | | | | | | | |
| Q | −1 | 1 | 0 | 0 | −3 | 5 | | | | | | | | | | | | | | |
| E | −1 | 0 | 0 | 2 | −4 | 2 | 5 | | | | | | | | | | | | | |
| G | 0 | −2 | 0 | −1 | −3 | −2 | −2 | 6 | | | | | | | | | | | | |
| H | −2 | 0 | 1 | −1 | −3 | 0 | 0 | −2 | 8 | | | | | | | | | | | |
| I | −1 | −3 | −3 | −3 | −1 | −3 | −3 | −4 | −3 | 4 | | | | | | | | | | |
| L | −1 | −2 | −3 | −4 | −1 | −2 | −3 | −4 | −3 | 2 | 4 | | | | | | | | | |
| K | −1 | 2 | 0 | −1 | −3 | 1 | 1 | −2 | −1 | −3 | −2 | 5 | | | | | | | | |
| M | −1 | −1 | −2 | −3 | −1 | 0 | −2 | −3 | −2 | 1 | 2 | −1 | 5 | | | | | | | |
| F | −2 | −3 | −3 | −3 | −2 | −3 | −3 | −3 | −1 | 0 | 0 | −3 | 0 | 6 | | | | | | |
| P | −1 | −2 | −2 | −1 | −3 | −1 | −1 | −2 | −2 | −3 | −3 | −1 | −2 | −4 | 7 | | | | | |
| S | 1 | −1 | 1 | 0 | −1 | 0 | 0 | 0 | −1 | −2 | −2 | 0 | −1 | −2 | −1 | 4 | | | | |
| T | 0 | −1 | 0 | −1 | −1 | −1 | −1 | −2 | −2 | −1 | −1 | −1 | −1 | −2 | −1 | 1 | 5 | | | |
| W | −3 | −3 | −4 | −4 | −2 | −2 | −3 | −2 | −2 | −3 | −2 | −3 | −1 | 1 | −4 | −3 | −2 | 11 | | |
| Y | −2 | −2 | −2 | −3 | −2 | −1 | −2 | −3 | 2 | −1 | −1 | −2 | −1 | 3 | −3 | −2 | −2 | 2 | 7 | |
| V | 0 | −3 | −3 | −3 | −1 | −2 | −2 | −3 | −3 | 3 | 1 | −2 | 1 | −1 | −2 | −2 | 0 | −3 | −1 | 4 |

Sequence identity of polynucleotide molecules is determined by similar methods using a ratio as disclosed above.

Those skilled in the art appreciate that there are many established algorithms available to align two amino acid sequences. The “FASTA” similarity search algorithm of Pearson and Lipman is a suitable protein alignment method for examining the level of identity shared by an amino acid sequence disclosed herein and the amino acid sequence of a putative variant ZSMF-16. The FASTA algorithm is described by Pearson and Lipman, Proc. Nat. Acad. Sci. USA 85:2444, 1988), and by Pearson, Meth. Enzymol. 183:63, 1990).

Briefly, FASTA first characterizes sequence similarity by identifying regions shared by the query sequence (e.g., SEQ ID NO:2) and a test sequence that have either the highest density of identities (if the ktup variable is 1) or pairs of identities (if ktup=2), without considering conservative amino acid substitutions, insertions, or deletions. The ten regions with the highest density of identities are then re-scored by comparing the similarity of all paired amino acids using an amino acid substitution matrix, and the ends of the regions are “trimmed” to include only those residues that contribute to the highest score. If there are several regions with scores greater than the “cutoff” value (calculated by a predetermined formula based upon the length of the sequence and the ktup value), then the trimmed initial regions are examined to determine whether the regions can be joined to form an approximate alignment with gaps. Finally, the highest scoring regions of the two amino acid sequences are aligned using a modification of the Needleman-Wunsch-Sellers algorithm (Needleman and Wunsch, J. Mol. Biol. 48:444, 1970; Sellers, SIAM J. Appl. Math. 26:787, 1974), which allows for amino acid insertions and deletions. Preferred parameters for FASTA analysis are: ktup=1, gap opening penalty=10, gap extension penalty=1, and substitution matrix=BLOSUM62, with other FASTA parameters set as default. These parameters can be introduced into a FASTA program by modifying the scoring matrix file (“SMATRIX”), as explained in Appendix 2 of Pearson, Meth. Enzymol. 183:63, 1990.

FASTA can also be used to determine the sequence identity of nucleic acid molecules using a ratio as disclosed above. For nucleotide sequence comparisons, the ktup value can range between one to six, preferably from three to six, most preferably three, with other FASTA parameters set as default.

The present invention includes nucleic acid molecules that encode a polypeptide having one or more conservative amino acid changes, compared with the amino acid sequence of SEQ ID NO:2. The BLOSUM62 table is an amino acid substitution matrix derived from about 2,000 local multiple alignments of protein sequence segments, representing highly conserved regions of more than 500 groups of related proteins (Henikoff and Henikoff, Proc. Nat. Acad. Sci. USA 89:10915 (1992)). Accordingly, the BLOSUM62 substitution frequencies can be used to define conservative amino acid substitutions that may be introduced into the amino acid sequences of the present invention. As used herein, the language “conservative amino acid substitution” refers to a substitution represented by a BLOSUM62 value of greater than −1. For example, an amino acid substitution is conservative if the substitution is characterized by a BLOSUM62 value of 0, 1, 2, or 3. Preferred conservative amino acid substitutions are characterized by a BLOSUM62 value of at least 1 (e.g., 1, 2 or 3), while more preferred conservative amino acid substitutions are characterized by a BLOSUM62 value of at least 2 (e.g., 2 or 3).

Variant ZSMF-16 polypeptides or substantially homologous ZSMF-16 polypeptides are characterized as having one or more amino acid substitutions, deletions or additions. These changes are preferably of a minor nature, that is conservative amino acid substitutions (see Table 4) and other substitutions that do not significantly affect the folding or activity of the protein or polypeptide; small deletions, typically of one to about 30 amino acids; and small amino- or carboxyl-terminal extensions, such as an amino-terminal methionine residue, a small linker peptide of up to about 20-25 residues, or an affinity tag. The present invention thus includes polypeptides of from about 424 amino acid residues to about 800 amino acid residues, that comprise a sequence that is at least 80%, preferably at least 90%, more preferably at least 95% or more identical to the corresponding region of SEQ ID NO:2, and more preferably having conserved cysteine residues corresponding to the amino acid residues 113, 131, 140, 167, 267, 291, 339, 379, 600 and 652 of SEQ ID NO:2. Polypeptides comprising affinity tags can further comprise a proteolytic cleavage site between the ZSMF-16 polypeptide and the affinity tag. Preferred such sites include thrombin cleavage sites and factor Xa cleavage sites.

TABLE 4
Conservative amino acid substitutions

| | |
|---|---|
| Basic: | arginine |
| | lysine |
| | histidine |
| Acidic: | glutamic acid |
| | aspartic acid |
| Polar: | glutamine |
| | asparagine |
| Hydrophobic: | leucine |
| | isoleucine |
| | valine |
| Aromatic: | phenylalanine |
| | tryptophan |
| | tyrosine |
| Small: | glycine |
| | alanine |
| | serine |
| | threonine |
| | methionine |

The proteins of the present invention can also comprise non-naturally occurring amino acid residues. Non-naturally occurring amino acids include, without limitation, trans-3-methylproline, 2,4-methanoproline, cis-4-hydroxyproline, trans-4-hydroxyproline, N-methyl- glycine, allo-threonine, methylthreonine, hydroxyethylcysteine, hydroxyethylhomocysteine, nitroglutamine, homoglutamine, pipecolic acid, thiazolidine carboxylic acid, dehydroproline, 3 and 4-methylproline, 3,3-dimethylproline, tert-leucine, norvaline, 2-azaphenylalanine, 3-azaphenylalanine, 4-azaphenylalanine, and 4-fluoro-phenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell free system comprising an E. coli S30 extract and commercially available enzymes and other reagents. Proteins are purified by chromatography. See, for example, Robertson et al., J. Am. Chem. Soc. 113:2722, 1991; Ellman et al., Methods Enzymol. 202:301, 1991; Chung et al., Science 259:806-9, 1993; and Chung et al., Proc. Natl. Acad. Sci. USA 90:10145-9, 1993). In a second method, translation is carried out in Xenopus oocytes by microinjection of mutated mRNA and chemically aminoacylated suppressor tRNAs (Turcatti et al., J. Biol. Chem. 271:19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the protein in place of its natural counterpart. See, Koide et al., Biochem. 33:7470-6, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395-3, 1993).

A limited number of non-conservative amino acids, amino acids that are not encoded by the generic code, non-naturally occurring amino acids, and unnatural amino acid residues may be substituted for ZSMF-16 amino acid residues.

Essential amino acids in the polypeptides of the present invention can be identified according to procedures known in the art, such as site-directed mutagenesis or alanine-scanning mutagenesis (Cunningham and Wells, Science 244: 1081-5, 1989; Bass et al., Proc. Natl. Acad. Sci. USA 88:4498-502, 1991). In the latter technique, single alanine mutations are introduced at every residue in the molecule, and the resultant mutant molecules are tested for biological activity (e.g., collapase activity, cellular interaction) to identify amino acid residues that are critical to the activity of the molecule. Sites of ligand-receptor interaction can also be determined by physical analysis of structure, as determined by such techniques as nuclear magnetic resonance, crystallography, electron diffraction, or photoaffinity; in conjunction with mutation of putative contact site amino acids. See, for example, de Vos et al., Science 255:306-12, 1992; Smith et al., J. Mol. Biol. 224:899-904, 1992; Wlodaver et al., FEBS Lett. 309:59-64, 1992. The identities of essential amino acids can also be inferred from analysis of homologies with related semaphorin polypeptides.

Determination of amino acid residues that are within regions or domains that are critical to maintaining structural integrity can be determined. Within these regions one can determine specific residues that will be more or less tolerant of change and maintain the overall tertiary structure of the molecule. Methods for analyzing sequence structure include, but are not limited to, alignment of multiple sequences with high amino acid or nucleotide identity and computer analysis using available software (e.g., the Insight II® viewer and homology modeling tools; MSI, San Diego, Calif.), secondary structure propensities, binary patterns, complementary packing and buried polar interactions (Barton, Current Opin. Struct. Biol. 5:372-376, 1995 and Cordes et al., Current Opin. Struct. Biol. 6:3-10, 1996). In general, when designing modifications to molecules or identifying specific fragments determination of structure will be accompanied by evaluating activity of modified molecules.

Amino acid sequence changes are made in ZSMF-16 polypeptides so as to minimize disruption of higher order structure essential to biological activity. For example, when the ZSMF-16 polypeptide comprises one or more helices, changes in amino acid residues will be made so as not to disrupt the helix geometry and other components of the molecule where changes in conformation abate some critical function, for example, binding of the molecule to its binding partners. The effects of amino acid sequence changes can be predicted by, for example, computer modeling as disclosed above or determined by analysis of crystal structure (see, e.g., Lapthorn et al., Nat. Struct. Biol. 2:266-268, 1995). Other techniques that are well known in the art compare folding of a variant protein to a standard molecule (e.g., the native protein). For example, comparison of the cysteine pattern in a variant and standard molecules can be made. Mass spectrometry and chemical modification using reduction and alkylation provide methods for determining cysteine residues which are associated with disulfide bonds or are free of such associations (Bean et al., Anal. Biochem. 201:216-226, 1992; Gray, Protein Sci. 2:1732-1748, 1993; and Patterson et al., Anal. Chem. 66:3727-3732, 1994). It is generally believed that if a modified molecule does not have the same disulfide bonding pattern as the standard molecule folding would be affected. Another well known and accepted method for measuring folding is circular dichrosism (CD). Measuring and comparing the CD spectra generated by a modified molecule and standard molecule is routine (Johnson, Proteins 7:205-214, 1990). Crystallography is another well known method for analyzing folding and structure. Nuclear magnetic resonance (NMR), digestive peptide mapping and epitope mapping are also known methods for analyzing folding and structural similarities between proteins and polypeptides (Schaanan et al., Science 257:961-964, 1992).

A Hopp/Woods hydrophilicity profile of the ZSMF-16 protein sequence as shown in SEQ ID NO:2 can be generated (Hopp et al., Proc. Natl. Acad. Sci.78:3824-3828, 1981; Hopp, J. Immun. Meth. 88:1-18, 1986 and Triquier et al., Protein Engineering 11:153-169, 1998). The profile is based on a sliding six-residue window. Buried G, S, and T residues and exposed H, Y, and W residues were ignored (see, Figure). For example, in ZSMF-16, hydrophilic regions include: (1) amino acid number 82 (Asp) to amino acid number 87 (Arg) of SEQ ID NO:2; (2) amino acid number 234 (Asp) to amino acid number 239 (Lys) of SEQ ID NO:2; (3) amino acid number 395 (Lys) to amino acid number 400 (Glu) of SEQ ID NO:2; (4) amino acid number 553 (Lys) to amino acid number 558 (Arg) of SEQ ID NO:2; and (5) amino acid number 683 (Glu) to amino acid number 688 (Glu) of SEQ ID NO:2.

Those skilled in the art will recognize that hydrophilicity or hydrophobicity will be taken into account when designing modifications in the amino acid sequence of a ZSMF-16 polypeptide, so as not to disrupt the overall structural and biological profile. Of particular interest for replacement are hydrophobic residues selected from the group consisting of Val, Leu and Ile or the group consisting of Met, Gly, Ser, Ala, Tyr and Trp. For example, residues tolerant of substitution could include those hydrophobic residues as shown in SEQ ID NO:2. Cysteine residues at positions 113, 131, 140, 167, 267, 291, 339, 379, 600 and 652 of SEQ ID NO:2 will be relatively intolerant of substitution.

The identities of essential amino acids can also be inferred from analysis of sequence similarity between semaphorin family members with ZSMF-16. Using methods such as “FASTA” analysis described previously, regions of high similarity are identified within a family of proteins and used to analyze amino acid sequence for conserved regions. An alternative approach to identifying a variant ZSMF-16 polynucleotide on the basis of structure is to determine whether a nucleic acid molecule encoding a potential variant ZSMF-16 polynucleotide can hybridize to a nucleic acid molecule having the nucleotide sequence of SEQ ID NO:1, as discussed above.

Other methods of identifying essential amino acids in the polypeptides of the present invention are procedures known in the art, such as site-directed mutagenesis or alanine-scanning mutagenesis (Cunningham and Wells, Science 244:1081 (1989), Bass et al., Proc. Natl Acad. Sci. USA 88:4498 (1991), Coombs and Corey, “Site-Directed Mutagenesis and Protein Engineering,” in Proteins: Analysis and Design, Angeletti (ed.), pages 259-311 (Academic Press, Inc. 1998)). In the latter technique, single alanine mutations are introduced at every residue in the molecule, and the resultant mutant molecules are tested for biological activity as disclosed below to identify amino acid residues that are critical to the activity of the molecule. See also, Hilton et al., J. Biol. Chem. 271:4699 (1996).

The present invention also includes functional fragments of ZSMF-16 polypeptides and nucleic acid molecules encoding such functional fragments. A “functional” ZSMF-16 or fragment thereof defined herein is characterized by its collapsin/semaphorin activity, proliferative or differentiating activity, by its ability to induce or inhibit specialized cell functions, or by its ability to bind specifically to an anti-ZSMF-16antibody or ZSMF-16 receptor (either soluble or immobilized). As previously described herein, ZSMF-16 is characterized by a sema domain structure and Ig-like domain, basic domain and other domains and motifs as described herein. Thus, the present invention further provides fusion proteins encompassing: (a) polypeptide molecules comprising one or more of the domains described above; and (b) functional fragments comprising one or more of these domains. The other polypeptide portion of the fusion protein may be contributed by another semaphorin, or by a non-native and/or an unrelated secretory signal peptide that facilitates secretion of the fusion protein.

Routine deletion analyses of nucleic acid molecules can be performed to obtain functional fragments of a nucleic acid molecule that encodes a ZSMF-16 polypeptide. For example, DNA molecules having the nucleotide sequence of SEQ ID NO:1 or fragments thereof, can be digested with Bal31 nuclease to obtain a series of nested deletions. These DNA fragments are then inserted into expression vectors in proper reading frame, and the expressed polypeptides are isolated and tested for ZSMF-16 activity, or for the ability to bind anti-ZSMF-16 antibodies or ZSMF-16 receptor. One alternative to exonuclease digestion is to use oligonucleotide-directed mutagenesis to introduce deletions or stop codons to specify production of a desired ZSMF-16 fragment. Alternatively, particular fragments of a ZSMF-16 polynucleotide can be synthesized using the polymerase chain reaction.

Standard methods for identifying functional domains are well-known to those of skill in the art. For example, studies on the truncation at either or both termini of interferons have been summarized by Horisberger and Di Marco, Pharmac. Ther. 66:507 (1995). Moreover, standard techniques for functional analysis of proteins are described by, for example, Treuter et al., Molec. Gen. Genet. 240:113 (1993); Content et al., “Expression and preliminary deletion analysis of the 42 kDa 2-5A synthetase induced by human interferon,” in Biological Interferon Systems, Proceedings of ISIR-TNO Meeting on Interferon Systems, Cantell (ed.), pages 65-72 (Nijhoff 1987); Herschman, “The EGF Receptor,” in Control of Animal Cell Proliferation J, Boynton et al., (eds.) pages 169-199 (Academic Press 1985); Coumailleau et al., J. Biol. Chem. 270:29270 (1995); Fukunaga et al., J. Biol. Chem. 270:25291 (1995); Yamaguchi et al., Biochem. Pharmacol. 50:1295 (1995); and Meisel et al., Plant Molec. Biol. 30:1 (1996).

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

Variants of the disclosed ZSMF-16 DNA and polypeptide sequences can be generated through DNA shuffling as disclosed by Stemmer, Nature 370:389-91, 1994 and Stemmer, Proc. Natl. Acad. Sci. USA 91:10747-51, 1994. Briefly, variant DNAs are generated by in vitro homologous recombination by random fragmentation of a parent DNA followed by reassembly using PCR, resulting in randomly introduced point mutations. This technique can be modified by using a family of parent DNAs, such as allelic variants or DNAs from different species, to introduce additional variability into the process. Selection or screening for the desired activity, followed by additional iterations of mutagenesis and assay provides for rapid “evolution” of sequences by selecting for desirable mutations while simultaneously selecting against detrimental changes.

Mutagenesis methods as disclosed herein can be combined with high-throughput screening methods to detect activity of cloned, mutagenized polypeptides in host cells. Mutagenized DNA molecules that encode active ligands or portions thereof (e.g., receptor-binding fragments) can be recovered from the host cells and rapidly sequenced using modern equipment. These methods allow the rapid determination of the importance of individual amino acid residues in a polypeptide of interest, and can be applied to polypeptides of unknown structure.

Using the methods discussed herein, one of ordinary skill in the art can identify and/or prepare a variety of polypeptides that are substantially similar to amino acid residues 23 (Gly) to 779 (Thr), or amino acid residues 76 (Leu) to 100 (Arg) of SEQ ID NO:2 or allelic variants thereof and retain the properties of the wild-type ZSMF-16 protein. Such polypeptides may include additional amino acids from the signal peptide, N-terminal region, semaphorin domain, middle domain, Ig-like domain, C-terminal domain, basic domain and other domains and motifs as described herein; the secretory signal sequence; affinity tags; and the like. Such polypeptides may also include additional polypeptide segments as generally disclosed herein.

The present invention further provides a variety of polypeptide fusions. For example, a ZSMF-16 polypeptide can be prepared as a fusion to a dimerizing protein as disclosed in U.S. Pat. Nos. 5,155,027 and 5,567,584. Preferred dimerizing proteins in this regard include immunoglobulin constant region domains. Immunoglobulin-ZSMF-16 polypeptide fusions can be expressed in genetically engineered cells to produce a variety of multimeric ZSMF-16 analogs. Auxiliary domains can be fused to ZSMF-16 polypeptides to target them to specific cells, tissues, or macromolecules. For example, a ZSMF-16 polypeptide or protein could be targeted to a predetermined cell type by fusing a ZSMF-16 polypeptide to a ligand that specifically binds to a receptor on the surface of the target cell. In this way, polypeptides and proteins can be targeted for therapeutic or diagnostic purposes. A ZSMF-16 polypeptide can be fused to two or more moieties, such as an affinity tag for purification and a targeting domain. Polypeptide fusions can also comprise one or more cleavage sites, particularly between domains. See, Tuan et al., Connective Tissue Research 34:1-9, 1996.

For any ZSMF-16 polypeptide, including variants and fusion proteins, one of ordinary skill in the art can readily generate a fully degenerate polynucleotide sequence encoding that variant using the information set forth in Tables 1 and 2 above.

The semaphorin polypeptides of the present invention, including full-length polypeptides, fragments (e.g., receptor-binding fragments, growth cone directing fragments, immune response provoking fragments), and fusion polypeptides, can be produced in genetically engineered host cells according to conventional techniques. Suitable host cells are those cell types that can be transformed or transfected with exogenous DNA and grown in culture, and include bacteria, fungal cells, and cultured higher eukaryotic cells. Eukaryotic cells, particularly cultured cells of multicellular organisms, are preferred. Techniques for manipulating cloned DNA molecules and introducing exogenous DNA into a variety of host cells are disclosed by Sambrook et al., Molecular Cloning: A Laboratory Manual, Second Edition, Cold Spring Harbor, N.Y., 1989; and Ausubel et al., eds., Current Protocols in Molecular Biology, John Wiley and Sons, Inc., N.Y., 1987.

In general, a DNA sequence encoding a ZSMF-16 polypeptide is operably linked to other genetic elements required for its expression, generally including a transcription promoter and terminator, within an expression vector. The vector will also commonly contain one or more selectable markers and one or more origins of replication, although those skilled in the art will recognize that within certain systems selectable markers may be provided on separate vectors, and replication of the exogenous DNA may be provided by integration into the host cell genome. Selection of promoters, terminators, selectable markers, vectors and other elements is a matter of routine design within the level of ordinary skill in the art. Many such elements are described in the literature and are available through commercial suppliers.

To direct a ZSMF-16 polypeptide into the secretory pathway of a host cell, a secretory signal sequence (also known as a leader sequence, prepro sequence or pre sequence) is provided in the expression vector. The secretory signal sequence may be that of the ZSMF-16 polypeptide (amino acid residues 1 (Met) through amino acid residue 22 (Ser) of SEQ ID NO:2), or may be derived from another secreted protein (e.g., t-PA) or synthesized de novo. The secretory signal sequence is joined to the ZSMF-16 DNA sequence in the correct reading frame. Secretory signal sequences are commonly positioned 5′ to the DNA sequence encoding the polypeptide of interest, although certain secretory signal sequences may be positioned elsewhere in the DNA sequence of interest (see, e.g., Welch et al., U.S. Pat. No. 5,037,743; Holland et al., U.S. Pat. No. 5,143,830).

Alternatively, the secretory signal sequence contained in the polypeptides of the present invention is used to direct other polypeptides into the secretory pathway. The present invention provides for such fusion polypeptides. A signal fusion polypeptide can be made wherein a secretory signal sequence derived from amino acid 1 (Met) to amino acid 22 (Ser) of SEQ ID NO:2 is operably linked to another polypeptide using methods known in the art and disclosed herein. The secretory signal sequence contained in the fusion polypeptides of the present invention is preferably fused amino-terminally to an additional peptide to direct the additional peptide into the secretory pathway. Such constructs have numerous applications known in the art. For example, these novel secretory signal sequence fusion constructs can direct the secretion of an active component of a normally non-secreted protein. Such fusions may be used in vivo or in vitro to direct peptides through the secretory pathway.

Moreover, using methods described in the art, polypeptide fusions, or hybrid ZSMF-16 proteins, are constructed using regions or domains of the inventive ZSMF-16 in combination with those of other Semaphorin family proteins (e.g. semaphorin IV or V, or chicken semaphorin 2, and the like), or heterologous proteins (Sambrook et al., ibid.; Altschul et al., ibid.; Picard, Cur. Opin. Biology, 5:511-5, 1994, and references therein). These methods allow the determination of the biological importance of larger domains or regions in a polypeptide of interest. Such hybrids may alter reaction kinetics, binding, constrict or expand the substrate specificity, alter activity in neurite assays, alter immune response, or gene transcription in a cell, alter cytoskeletal organization and cell motility, transformation, or invasiveness, or alter tissue and cellular localization of a polypeptide, and can be applied to polypeptides of unknown structure.

Fusion proteins can be prepared by methods known to those skilled in the art by preparing each component of the fusion protein and chemically conjugating them. Alternatively, a polynucleotide encoding various components of the fusion protein in the proper reading frame can be generated using known techniques and expressed by the methods described herein. For example, part or all of a domain(s) conferring a structural or biological function may be swapped between ZSMF-16 of the present invention with the functionally equivalent domain(s) from another family member. Such domains include, but are not limited to, signal peptide, N-terminal region, semaphorin domain, middle domain, Ig-like domain, C-terminal domain, basic domain and other domains and motifs as described herein. Such fusion proteins would be expected to have a biological functional profile that is the same or similar to polypeptides of the present invention or other known semaphorin family proteins (e.g. affecting neurite growth or collapsing activity, and the like) depending on the fusion constructed. Moreover, such fusion proteins may exhibit other properties as disclosed herein.

Standard molecular biological and cloning techniques can be used to swap the equivalent domains between the ZSMF-16 polypeptide and those polypeptides to which they are fused. Generally, a DNA segment that encodes a domain of interest, e.g., a ZSMF-16 active polypeptide or motif described herein, is operably linked in frame to at least one other DNA segment encoding an additional polypeptide and inserted into an appropriate expression vector, as described herein. Generally DNA constructs are made such that the several DNA segments that encode the corresponding regions of a polypeptide are operably linked in frame to make a single construct that encodes the entire fusion protein, or a functional portion thereof. For example, a DNA construct would encode from N-terminus to C-terminus a fusion protein comprising a signal polypeptide followed by a mature polypeptide; or a DNA construct would encode from N-terminus to C-terminus a fusion protein comprising an N-terminal region followed by a sema domain; or a DNA construct would encode from N-terminus to C-terminus a fusion protein comprising a sema domain followed by an Ig-like domain ; or a DNA construct would encode from N-terminus to C-terminus a fusion protein comprising a signal peptide, N-terminal region, semaphorin domain, middle domain, Ig-like domain, C-terminal domain, and a basic domain; or for example, any of the above as interchanged with equivalent regions from another semaphorin family protein. Such fusion proteins can be expressed, isolated, and assayed for activity as described herein. Moreover, such fusion proteins can be used to express and secrete fragments of the ZSMF-16 polypeptide, to be used, for example to inoculate an animal to generate anti-ZSMF-16 antibodies as described herein. For example a secretory signal sequence can be operably linked to the N-terminal region, semaphorin domain, middle domain, Ig-like domain, C-terminal domain, a basic domain, or a combination thereof (e.g., operably linked polypeptides comprising the N-terminal region fused to the sema domain, or ZSMF-16 polypeptide fragments described herein), to secrete a fragment of ZSMF-16 polypeptide that can be purified as described herein and serve as an antigen to be inoculated into an animal to produce anti-ZSMF-16 antibodies, as described herein.

In addition, the proteins of the present invention (or polypeptide fragments thereof) can be joined to other bioactive molecules, particularly other semaphorins, to provide multi-functional molecules. For example, one or more domains from ZSMF-16 can be joined to other semaphorins to enhance their biological properties or efficiency of production.

The present invention thus provides a series of novel, hybrid molecules in which a segment comprising one or more of the domains of ZSMF-16 is fused to another polypeptide. Fusion is preferably done by splicing at the DNA level, as described herein, to allow expression of chimeric molecules in recombinant production systems. The resultant molecules are then assayed for such properties as enhanced or diminished neurite collapsing activity, increased or decreased immune response activity, improved solubility, improved stability, prolonged clearance half-life, improved expression and secretion levels, and pharmacodynamics. Such hybrid molecules may further comprise additional amino acid residues (e.g. a polypeptide linker) between the component proteins or polypeptides.

Cultured mammalian cells are suitable hosts within the present invention. Methods for introducing exogenous DNA into mammalian host cells include calcium phosphate-mediated transfection (Wigler et al., Cell 14:725, 1978; Corsaro and Pearson, Somatic Cell Genetics 7:603, 1981; Graham and Van der Eb, Virology 52:456, 1973), electroporation (Neumann et al., EMBO J. 1:841-5, 1982), DEAE-dextran mediated transfection (Ausubel et al., ibid.), and liposome-mediated transfection (Hawley-Nelson et al., Focus 15:73, 1993; Ciccarone et al., Focus 15:80, 1993). The production of recombinant polypeptides in cultured mammalian cells is disclosed, for example, by Levinson et al., U.S. Pat. No. 4,713,339; Hagen et al., U.S. Pat. No. 4,784,950; Palmiter et al., U.S. Pat. No. 4,579,821; and Ringold, U.S. Pat. No. 4,656,134. Suitable cultured mammalian cells include the COS-1 (ATCC No. CRL 1650), COS-7 (ATCC No. CRL 1651), BHK (ATCC No. CRL 1632), BHK 570 (ATCC No. CRL 10314), 293 (ATCC No. CRL 1573; Graham et al., J. Gen. Virol. 36:59-72, 1977), Chinese hamster ovary (e.g., CHO-K1; ATCC No. CCL 61) cell lines and DG44 CHO cells (Chasin et al., Som. Cell. Molec. Genet. 12:555-66, 1986). Additional suitable cell lines are known in the art and available from public depositories such as the American Type Culture Collection, Rockville, Md. In general, strong transcription promoters are preferred, such as promoters from SV-40 or cytomegalovirus. See, e.g., U.S. Pat. No. 4,956,288. Other suitable promoters include those from metallothionein genes (U.S. Pat. Nos. 4,579,821 and 4,601,978) and the adenovirus major late promoter.

Drug selection is generally used to select for cultured mammalian cells into which foreign DNA has been inserted. Such cells are commonly referred to as “transfectants”. Cells that have been cultured in the presence of the selective agent and are able to pass the gene of interest to their progeny are referred to as “stable transfectants.” A preferred selectable marker is a gene encoding resistance to the antibiotic neomycin. Selection is carried out in the presence of a neomycin-type drug, such as G-418 or the like. Selection systems may also be used to increase the expression level of the gene of interest, a process referred to as “amplification.” Amplification is carried out by culturing transfectants in the presence of a low level of the selective agent and then increasing the amount of selective agent to select for cells that produce high levels of the products of the introduced genes. A preferred amplifiable selectable marker is dihydrofolate reductase, which confers resistance to methotrexate. Other drug resistance genes (e.g., hygromycin resistance, multi-drug resistance, puromycin acetyltransferase) can also be used.

In a preferred embodiment ZSMF-16 DNA fragments are subcloned into mammalian expression plasmids, such as pZP9 (ATCC No. 98668) or modifications thereof. For expression of affinity tagged ZSMF-16 proteins, Glu—Glu-tagged for example, such expression plasmids contain the mouse metallothionein-1 promoter; a TPA leader peptide followed by the sequence encoding a Glu—Glu tag (e.g., SEQ ID NO:4), for expression of N-terminal Glu—Glu ZSMF-16 proteins; the ZSMF-16 polynucleotide sequence without the native signal sequence, and a human growth hormone terminator. For expression of C-terminal Glu—Glu tagged proteins the expression cassette can be modified to place the sequence encoding a Glu—Glu tag (e.g., SEQ ID NO:4) after the ZSMF-16 nucleotide sequence followed by a stop codon and the human growth hormone terminator. Within one preferred embodiment, such expression vectors would be transfected and expressed in mammalian cells, such as BHK or CHO cells. Transformed cells can be screened for expression of ZSMF-16 proteins by filter assay. Affinity tagged proteins can be detected using conjugated antibodies to the tag, such as anti-Glu—Glu antibody-HRP conjugate. Colonies expressing ZSMF-16 can be selected and subjected to Western Blot analysis and mycoplasma testing. Preferably individual clones can be expanded and used for large scale production of ZSMF-16 proteins.

Other higher eukaryotic cells can also be used as hosts, including plant cells, insect cells and avian cells. The use of Agrobacterium rhizogenes as a vector for expressing genes in plant cells has been reviewed by Sinkar et al., J. Biosci. (Bangalore) 11:47-58, 1987. Transformation of insect cells and production of foreign polypeptides therein is disclosed by Guarino et al., U.S. Pat. No. 5,162,222 and WIPO publication WO 94/06463. Insect cells can be infected with recombinant baculovirus, commonly derived from Autographa californica nuclear polyhedrosis virus (AcNPV). See, King, L. A. and Possee, R. D., The Baculovirus Expression System: A Laboratory Guide, London, Chapman & Hall; O'Reilly, D. R. et al., Baculovirus Expression Vectors: A Laboratory Manual, New York, Oxford University Press., 1994; and, Richardson, C. D., Ed., Baculovirus Expression Protocols. Methods in Molecular Biology, Totowa, N.J., Humana Press, 1995. The second method of making recombinant baculovirus utilizes a transposon-based system described by Luckow (Luckow, V. A, et al., J Virol 67:4566-79, 1993). This system is sold in the Bac-to-Bac™ kit (Life Technologies, Rockville, Md.). This system utilizes a transfer vector, pFastBac1™ (Life Technologies) containing a Tn7 transposon to move the DNA encoding the ZSMF-16 polypeptide into a baculovirus genome maintained in E. coli as a large plasmid called a “bacmid.” The pFastBac1™ transfer vector utilizes the AcNPV polyhedrin promoter to drive the expression of the gene of interest, in this case ZSMF-16. However, pFastBac1™ can be modified to a considerable degree. The polyhedrin promoter can be removed and substituted with the baculovirus basic protein promoter (also known as Pcor, p6.9 or MP promoter) which is expressed earlier in the baculovirus infection, and has been shown to be advantageous for expressing secreted proteins. See, Hill-Perkins, M. S. and Possee, R. D., J. Gen. Virol. 71:971-6, 1990; Bonning, B. C. et al., J. Gen. Virol. 75:1551-6, 1994; and, Chazenbalk, G. D., and Rapoport, B., J. Biol. Chem. 270:1543-9, 1995. In such transfer vector constructs, a short or long version of the basic protein promoter can be used. Moreover, transfer vectors can be constructed which replace the native ZSMF-16 secretory signal sequences with secretory signal sequences derived from insect proteins. For example, a secretory signal sequence from Ecdysteroid Glucosyltransferase (EGT), honey bee Melittin (Invitrogen, Carlsbad, Calif.), or baculovirus gp67 (PharMingen, San Diego, Calif.) can be used in constructs to replace the native ZSMF-16 secretory signal sequence. In addition, transfer vectors can include an in-frame fusion with DNA encoding an epitope tag at the C- or N-terminus of the expressed ZSMF-16 polypeptide, for example, a Glu—Glu epitope tag (Grussenmeyer, T. et al., Proc. Natl. Acad. Sci. 82:7952-4, 1985). Using a technique known in the art, a transfer vector containing ZSMF-16 is transformed into E. coli, and screened for bacmids which contain an interrupted lacZ gene indicative of recombinant baculovirus. The bacmid DNA containing the recombinant baculovirus genome is isolated, using common techniques, and used to transfect Spodoptera frugiperda cells, e.g. Sf9 cells. Recombinant virus that expresses ZSMF-16 is subsequently produced. Recombinant viral stocks are made by methods commonly used the art.

The recombinant virus is used to infect host cells, typically a cell line derived from the fall armyworm, Spodoptera frugiperda. See, in general, Glick and Pasternak, Molecular Biotechnology: Principles and Applications of Recombinant DNA, ASM Press, Washington, D.C., 1994. Another suitable cell line is the High FiveO™ cell line (Invitrogen) derived from Trichoplusia ni (U.S. Pat. No.5,300,435). Commercially available serum-free media are used to grow and maintain the cells. Suitable media are Sf900 II™ (Life Technologies) or ESF 921™ (Expression Systems) for the Sf9 cells; and Ex-cellO405™ (JRH Biosciences, Lenexa, Kans.) or Express FiveO™ (Life Technologies) for the T. ni cells. The cells are grown up from an inoculation density of approximately 2-5×10⁵ cells to a density of 1-2×10⁶ cells at which time a recombinant viral stock is added at a multiplicity of infection (MOI) of 0.1 to 10, more typically near 3. Procedures used are generally described in available laboratory manuals (King, L. A. and Possee, R. D., ibid.; O'Reilly, D. R. et al., ibid.; Richardson, C. D., ibid.). Subsequent purification of the ZSMF-16 polypeptide from the supernatant can be achieved using methods described herein.

Fungal cells, including yeast cells, and particularly cells of the genus Saccharomyces, can also be used within the present invention, such as for producing fragments or polypeptide fusions. Methods for transforming yeast cells with exogenous DNA and producing recombinant polypeptides therefrom are disclosed by, for example, Kawasaki, U.S. Pat. No. 4,599,311; Kawasaki et al., U.S. Pat. No. 4,931,373; Brake, U.S. Pat. No. 4,870,008; Welch et al., U.S. Pat. No. 5,037,743; and Murray et al., U.S. Pat. No. 4,845,075. Transformed cells are selected by phenotype determined by the selectable marker, commonly drug resistance or the ability to grow in the absence of a particular nutrient (e.g., leucine). A preferred vector system for use in yeast is the POT1 vector system disclosed by Kawasaki et al. (U.S. Pat. No. 4,931,373), which allows transformed cells to be selected by growth in glucose-containing media. Suitable promoters and terminators for use in yeast include those from glycolytic enzyme genes (see, e.g., Kawasaki, U.S. Pat. No. 4,599,311; Kingsman et al., U.S. Pat. No. 4,615,974; and Bitter, U.S. Pat. No. 4,977,092) and alcohol dehydrogenase genes. See also U.S. Pat. Nos. 4,990,446; 5,063,154; 5,139,936 and 4,661,454. Transformation systems for other yeasts, including Hansenula polymorpha, Schizosaccharomyces pombe, Kluyveromyces lactis, Kluyveromyces fragilis, Ustilago maydis, Pichia pastoris, Pichia methanolica, Pichia guillermondii and Candida maltosa are known in the art. See, for example, Gleeson et al., J. Gen. Microbiol. 132:3459-65, 1986; and Cregg, U.S. Pat. No. 4,882,279. Aspergillus cells may be utilized according to the methods of McKnight et al., U.S. Pat. No. 4,935,349. Methods for transforming Acremonium chrysogenum are disclosed by Sumino et al., U.S. Pat. No. 5,162,228. Methods for transforming Neurospora are disclosed by Lambowitz, U.S. Pat. No. 4,486,533.

The use of Pichia methanolica as host for the production of recombinant proteins is disclosed in WIPO Publication WO 9717450. DNA molecules for use in transforming P. methanolica will commonly be prepared as double-stranded, circular plasmids, which are preferably linearized prior to transformation. For polypeptide production in P. methanolica, it is preferred that the promoter and terminator in the plasmid be that of a P. methanolica gene. A preferred promoter is that of a P. methanolica alcohol utilization gene (AUGI). P. methanolica contains a second alcohol utilization gene, AUG2, the promoter of which can also be used. Other useful promoters include those of the dihydroxyacetone synthase (DHAS), formate dehydrogenase (FMD), and catalase (CAT) genes. To facilitate integration of the DNA into the host chromosome, it is preferred to have the entire expression segment of the plasmid flanked at both ends by host DNA sequences. This is conveniently accomplished by including 3′ untranslated DNA sequence at the downstream end of the expression segment and relying on the promoter sequence at the 5′ end. When using linear DNA, the expression segment will be flanked by cleavage sites to allow for linearization of the molecule and separation of the expression segment from other sequences (e.g., a bacterial origin of replication and selectable marker). Preferred such cleavage sites are those that are recognized by restriction endonucleases that cut infrequently within a DNA sequence, such as those that recognize 8-base target sequences (e.g., Not I). A preferred selectable marker for use in Pichia methanolica is a P. methanolica ADE2 gene, which encodes phosphoribosyl-5-aminoimidazole carboxylase (AIRC; EC 4.1.1.21). The ADE2 gene, when transformed into an ade2 host cell, allows the cell to grow in the absence of adenine. Other nutritional markers that can be used include the P. methanolica ADE1, HIS3, and LEU2 genes, which allow for selection in the absence of adenine, histidine, and leucine, respectively. For large-scale, industrial processes where it is desirable to minimize the use of methanol, it is preferred to use host cells in which both methanol utilization genes (AUG1 and AUG2) are deleted. For production of secreted proteins, host cells deficient in vacuolar protease genes (PEP4 and PRBI) are preferred. Gene-deficient mutants can be prepared by known methods, such as site-directed mutagenesis. P. methanolica genes can be cloned on the basis of homology with their counterpart Saccharomyces cerevisiae genes.

Electroporation is used to facilitate the introduction of a plasmid containing DNA encoding a polypeptide of interest into P. methanolica cells. See, in general, Neumann et al., EMBO J. 1:841-5, 1982 and Meilhoc et al., Bio/Technology 8:223-7, 1990. For transformation of P. methanolica, electroporation is most efficient when the cells are exposed to an exponentially decaying, pulsed electric field having a field strength of from 2.5 to 4.5 kV/cm, preferably about 3.75 kV/cm, and a time constant (t) of from 1 to 40 milliseconds, most preferably about 20 milliseconds.

Prokaryotic host cells, including strains of the bacteria Escherichia coli, Bacillus and other genera are also useful host cells within the present invention. Techniques for transforming these hosts and expressing foreign DNA sequences cloned therein are well known in the art (see, e.g., Sambrook et al., ibid.). When expressing a ZSMF-16 polypeptide in bacteria such as E. coli, the polypeptide may be retained in the cytoplasm, typically as insoluble granules, or may be directed to the periplasmic space by a bacterial secretion sequence. In the former case, the cells are lysed, and the granules are recovered and denatured using, for example, guanidine isothiocyanate or urea. The denatured polypeptide can then be refolded and dimerized by diluting the denaturant, such as by dialysis against a solution of urea and a combination of reduced and oxidized glutathione, followed by dialysis against a buffered saline solution. In the latter case, the polypeptide can be recovered from the periplasmic space in a soluble and functional form by disrupting the cells (by, for example, sonication or osmotic shock) to release the contents of the periplasmic space and recovering the protein, thereby obviating the need for denaturation and refolding.

Transformed or transfected host cells are cultured according to conventional procedures in a culture medium containing nutrients and other components required for the growth of the chosen host cells. A variety of suitable media, including defined media and complex media, are known in the art and generally include a carbon source, a nitrogen source, essential amino acids, vitamins and minerals. Media may also contain such components as growth factors or serum, as required. The growth medium will generally select for cells containing the exogenously added DNA by, for example, drug selection or deficiency in an essential nutrient which is complemented by the selectable marker carried on the expression vector or co-transfected into the host cell. P. methanolica cells are cultured in a medium comprising adequate sources of carbon, nitrogen and trace nutrients at a temperature of about 25° C. to 35° C. Liquid cultures are provided with sufficient aeration by conventional means, such as shaking of small flasks or sparging of fermentors. A preferred culture medium for P. methanolica is YEPD.

Expressed recombinant ZSMF-16 polypeptides (or chimeric or fused ZSMF-16 polypeptides) can be purified using fractionation and/or conventional purification methods and media. Ammonium sulfate precipitation and acid or chaotrope extraction may be used for fractionation of samples. Exemplary purification steps may include hydroxyapatite, size exclusion, FPLC and reverse-phase high performance liquid chromatography. Suitable chromatographic media include derivatized dextrans, agarose, cellulose, polyacrylamide, specialty silicas, and the like. PEI, DEAE, QAE and Q derivatives are preferred. Exemplary chromatographic media include those media derivatized with phenyl, butyl, or octyl groups, such as Phenyl-Sepharose FF (Pharmacia), Toyopearl butyl 650 (Toso Haas, Montgomeryville, Pa.), Octyl-Sepharose (Pharmacia) and the like; or polyacrylic resins, such as Amberchrom CG 71 (Toso Haas) and the like. Suitable solid supports include glass beads, silica-based resins, cellulosic resins, agarose beads, cross-linked agarose beads, polystyrene beads, cross-linked polyacrylamide resins and the like that are insoluble under the conditions in which they are to be used. These supports may be modified with reactive groups that allow attachment of proteins by amino groups, carboxyl groups, sulfhydryl groups, hydroxyl groups and/or carbohydrate moieties. Examples of coupling chemistries include cyanogen bromide activation, N-hydroxysuccinimide activation, epoxide activation, sulfhydryl activation, hydrazide activation, and carboxyl and amino derivatives for carbodiimide coupling chemistries. These and other solid media are well known and widely used in the art, and are available from commercial suppliers. Methods for binding receptor polypeptides to support media are well known in the art. Selection of a particular method is a matter of routine design and is determined in part by the properties of the chosen support. See, for example, Affinity Chromatography: Principles & Methods, Pharmacia LKB Biotechnology, Uppsala, Sweden, 1988.

The polypeptides of the present invention can be isolated by exploitation of their physical or biochemical properties. For example, methods used to purify semaphorins are exemplary (See, Luo, Y. et al., Cell 75:217-227, 1993). Moreover, immobilized metal ion adsorption (IMAC) chromatography can be used to purify histidine-rich proteins, including those comprising polyhistidine tags. Briefly, a gel is first charged with divalent metal ions to form a chelate (Sulkowski, Trends in Biochem. 3:1-7, 1985). Histidine-rich proteins will be adsorbed to this matrix with differing affinities, depending upon the metal ion used, and will be eluted by competitive elution, lowering the pH, or use of strong chelating agents. Other methods of purification include purification of glycosylated proteins by lectin affinity chromatography and ion exchange chromatography (Methods in Enzymol., Vol. 182, “Guide to Protein Purification”, M. Deutscher, (ed.), Acad. Press, San Diego, 1990, pp.529-39). Within additional embodiments of the invention, a fusion of the polypeptide of interest and an affinity tag (e.g., maltose-binding protein, Glu—Glu tag, or an immunoglobulin domain) may be constructed to facilitate purification.

It is preferred to purify the protein to >80% purity, more preferably to >90% purity, even more preferably >95%, and particularly preferred is a pharmaceutically pure state, that is greater than 99.9% pure with respect to contaminating macromolecules, particularly other proteins and nucleic acids, and free of infectious and pyrogenic agents. Preferably, a purified protein is substantially free of other proteins, particularly other proteins of animal origin.

ZSMF-16 polypeptides or fragments thereof may also be prepared through chemical synthesis. ZSMF-16 polypeptides may be monomers or multimers; glycosylated or non-glycosylated; pegylated or non-pegylated; and may or may not include an initial methionine amino acid residue.

Polypeptides of the present invention can also be synthesized by exclusive solid phase synthesis, partial solid phase methods, fragment condensation or classical solution synthesis. Methods for synthesizing polypeptides are well known in the art. See, for example, Merrifield, J. Am. Chem. Soc. 85:2149, 1963; Kaiser et al., Anal. Biochem. 34:595, 1970. After the entire synthesis of the desired peptide on a solid support, the peptide-resin is with a reagent which cleaves the polypeptide from the resin and removes most of the side-chain protecting groups. Such methods are well established in the art.

Polypeptides containing the receptor-binding region of the ligand can be used for purification of receptor. The ligand polypeptide is immobilized on a solid support, such as beads of agarose, cross-linked agarose, glass, cellulosic resins, silica-based resins, polystyrene, cross-linked polyacrylamide, or like materials that are stable under the conditions of use. Methods for linking polypeptides to solid supports are known in the art, and include amine chemistry, cyanogen bromide activation, N-hydroxysuccinimide activation, epoxide activation, sulflhydryl activation, and hydrazide activation. The resulting media will generally be configured in the form of a column, and fluids containing receptors are passed through the column one or more times to allow receptor to bind to the ligand polypeptide. The receptor is then eluted using changes in salt concentration, chaotropic agents (MnCl₂), or pH to disrupt ligand-receptor binding.

ZSMF-16 polypeptides or ZSMF-16 fusion proteins are used, for example, to identify the ZSMF-16 receptor. Using labeled ZSMF-16 polypeptides, cells expressing the receptor are identified by fluorescence immunocytometry or immunohistochemistry. ZSMF-16 polypeptides are useful in determining the distribution of the receptor on tissues or specific cell lineages, and to provide insight into receptor/ligand biology. An exemplary method to identify a ZSMF-16 receptor in vivo or in vitro, e.g., in cell lines, is to us a ZSMF-16 polypeptide fused to the catalytic domain of Alkaline phosphatase (AP), as described in Feiner, L. et al., Neuron 19:539-545, 1997. Such AP fusions, as well as radiolabeled ZSMF-16, ZSMF-16 fusions with fluorescent lables, and others described herein, combined with standard cloning techniques enable one of skill in the art to visualize, identify and clone the ZSMF-16 receptor.

Semaphorins have been characterized as chemorepellants in the neurological system, responsible for directing neurite growth and neuronal system organization. Semaphorin polypeptides, agonists and antagonists can be used to modulate neurite growth and development and demarcate nervous system structures. Mutations deleting semaphorins result in axon projections in to inappropriate regions of the spinal cord. ZSMF-16 is likely expressed in various brain tissues and in spinal cord. ZSMF-16 polypeptides and ZSMF-16 antagonists, including anti-ZSMF-16 antibodies, would be useful as in treatment of peripheral neuropathies by increasing spinal cord and sensory neurite outgrowth and patterning by acting as repulsive and attractive guidance cues to the developing sensory or motor neuron. Guidance cues serve to direct or constrain the pattern of neuron growth, channeling axons to their appropriate destination. In the absence of guidance cues neuron growth is random and unstructured. As such, ZSMF-16 polypeptides, agonists, and antagonists, including anti-ZSMF-16 antibodies, can be included in the therapeutic treatment for the regeneration and direction of neurite outgrowths following strokes, brain damage caused by head injuries and paralysis caused by spinal injuries. Application may also be made in treating neurodegenerative diseases such as amyotrophic lateral sclerosis (ALS), Alzheimer's disease, Huntington's disease, Parkinson's disease and peripheral neuropathies, or demyelinating diseases including multiple sclerosis, by directing neuronal outgrowths. Such an application would be repair of transected axons that are common in lesions of multiple sclerosis (Trapp et al., N. Engl. J. Med. 338:278-85, 1998).

ZSMF-16 may be expressed in non-neuronal tissues but likely influences the development and innervation of these tissues. G-Sema I and collapsin are hypothesized to act in vivo as repulsive or inhibitory molecules that prevent neighboring ventral motorneurons from innervating extra thoracic muscle. In other situations, G-Sema I and collapsin may also act as an attractive agent to promote innervation (Kolodkin,A. L. et al., Cell 75:1389-99, 1993). ZSMF-16 polypeptides would be useful in directing neuronal development and innervation patterns in various tissues by acting as a guidance cue and stimulating the formation of normal synaptic terminal arborizations, for example on a target muscle tissue.

Moreover, semaphorin III, has been reported to play a role in the development of bones and heart by acting as a restraining signal during organ development (Behar et al., Nature 383:525-8, 1996). Similarly, ZSMF-16 would be useful in directing and defining the growth of developing tissue, in particular, defining the margins of a particular organ or tissue. ZSMF-16 polypeptides would be useful in the defining and directing development of various tissues and organs including those associated with muscle, fibroblasts, reproductive, endocrine and lymphatic tissues.

Semaphorins have also been associated with non-neuronal functions. Viral semaphorins have been speculated to act as modulators of the immune system, as natural immunosuppressants reducing the immune response by mimicking the function of a particular subfamily of semaphorins that can modulate immune functions (Kolodkin et al., ibid., and Ensser and Fleckenstein, ibid.). Other non-viral semaphorins are also associated with the immune system. Human semaphorin E, which is homologous to viral cytokine inhibiting proteins, contains conserved regions of amino acid residues that have been found in the viral semaphorins. Semaphorin E was found to be upregulated in rheumatoid synovial fibroblastoid cells which suggests that it may have a role as a regulator of inflammatory processes and an involvement in the development of rheumatoid arthritis (Mangasser-Stephan et al., Biochem. Biophys. Res. Comm. 234:153-6, 1997). Semaphorin CD100 has been reported to promote B-cell growth and aggregation and may be involved in lymphocyte activation (Hall et al., Proc. Natl. Acad. Sci. USA 93: 11780-5, 1996) and its mouse homologue, mSema G, is expressed on lymphocytes and is suggested to play a role in the immune system as well (Furuyama et al., J. Biol. Chem. 271:33376-81, 1996).

Moreover, as a semaphorin, ZSMF-16 may be a mediator of immunosuppression, in particular the activation and regulation of T lymphocytes. As such, ZSMF-16 polypeptides would be useful additions to therapies for treating immunodeficiencies. For example, ZSMF-16 can be useful in diagnosing and treating conditions where selective elimination of inappropriately activated T cells or other immune cells would be beneficial, such as in autoimmune diseases, in particular insulin dependent diabetes mellitus, rheumatoid arthritis and multiple sclerosis. Such polypeptides could be used to screen serum samples from patients suffering from such conditions in comparison to normal samples. Inappropriately activated T cells would include those specific for self-peptide/self-major histocompatability complexes and those specific for non-self antigens from transplanted tissues. Use could also be made of these polypeptides in blood screening for removal of inappropriately activated T cells before returning the blood to the donor. Those skilled in the art will recognize that conditions related to ZSMF-16 underexpression or overexpression may be amenable to treatment by therapeutic manipulation of ZSMF-16 protein levels.

ZSMF-16 polypeptides can be used in vivo as an anti-inflammatory, for inhibition of antigen in humoral and cellular immunity and for immunosuppression in graft and organ transplants. Methods of assessing ZSMF-16 pro- or anti-inflammatory effects are well known in the art.

ZSMF-16 polynucleotides and/or polypeptides can be used for regulating the proliferation and stimulation of a wide variety of cells, such as T cells, B cells, lymphocytes, peripheral blood mononuclear cells, fibroblasts and hematopoietic cells. ZSMF-16 polypeptides will also find use in mediating metabolic or physiological processes in vivo. Proliferation and differentiation can be measured in vitro using cultured cells. Suitable cell lines are available commercially from such sources as the American Type Culture Collection (Rockville, Md.). Bioassays and ELISAs are available to measure cellular response to ZSMF-16, in particular are those which measure changes in cytokine production as a measure of cellular response (see for example, Current Protocols in Immunology ed. John Coligan et al., NIH, 1996). Also of interest are apoptosis assays, such as the DNA fragmentation assay described by Wiley et al. (Immunity, 3:673-82, 1995, and the cell death assay described by Pan et al., Science, 276:111-13, 1997). Assays to measure other cellular responses, including antibody isotype, monocyte activation, NK cell formation and antigen presenting cell function are also known. The ZSMF-16 polypeptides may also be used to stimulate lymphocyte development, such as during bone marrow transplantation and as therapy for some cancers.

In vivo response to ZSMF-16 polypeptides can also be measured by administering polypeptides of the claimed invention to the appropriate animal model. Well established animal models are available to test in vivo efficacy of ZSMF-16 polypeptides for certain disease states. In particular, ZSMF-16 polypeptides can be tested in vivo in a number of animal models of autoimmune disease, such as the NOD mice, a spontaneous model system for insulin-dependent diabetes mellitus (IDDM), to study induction of non-responsiveness in the animal model. Administration of ZSMF-16 polypeptides prior to or after onset of disease can be monitored by assay of urine glucose levels in the NOD mouse. Alternatively, induced models of autoimmune disease, such as experimental allergic encephalitis (EAE), can be administered ZSMF-16 polypeptides. Administration in a preventive or intervention mode can be followed by monitoring the clinical symptoms of EAE. In addition, ZSMF-16 polypeptides can be tested in vivo in animal models for cancer, where suppression or apoptosis of introduced tumor cells can be monitored following administration of ZSMF-16.

As a ligand, the activity of ZSMF-16 polypeptide can be measured by a silicon-based biosensor microphysiometer which measures the extracellular acidification rate or proton excretion associated with receptor binding and subsequent physiologic cellular responses. An exemplary device is the Cytosensor™ Microphysiometer (Molecular Devices, Sunnyvale, Calif.). A variety of cellular responses, such as cell proliferation, ion transport, energy production, inflammatory response, regulatory and receptor activation, and the like, can be measured by this method. See, for example, McConnell et al., Science 257:1906-12, 1992; Pitchford et al., Meth. Enzymol. 228:84-108, 1997; Arimilli et al., J. Immunol. Meth. 212:49-59, 1998; Van Liefde et al., Eur. J. Pharmacol. 346:87-95, 1998. The microphysiometer can be used for assaying adherent or non-adherent eukaryotic or prokaryotic cells. By measuring extracellular acidification changes in cell media over time, the microphysiometer directly measures cellular responses to various stimuli, including ZSMF-16 polypeptide, its agonists, or antagonists. Preferably, the microphysiometer is used to measure responses of a ZSMF-16-responsive eukaryotic cell, compared to a control eukaryotic cell that does not respond to ZSMF-16 polypeptide. ZSMF-16-responsive eukaryotic cells comprise cells into which a receptor for ZSMF-16 has been transfected creating a cell that is responsive to ZSMF-16; or cells naturally responsive to ZSMF-16 such as cells derived from neurological, endrocrinological or tumor tissue. Differences, measured by a change, for example, an increase or diminution in extracellular acidification, in the response of cells exposed to ZSMF-16 polypeptide, relative to a control not exposed to ZSMF-16, are a direct measurement of ZSMF-16-modulated cellular responses. Moreover, such ZSMF-16-modulated responses can be assayed under a variety of stimuli. Using the microphysiometer, there is provided a method of identifying agonists of ZSMF-16 polypeptide, comprising providing cells responsive to a ZSMF-16 polypeptide, culturing a first portion of the cells in the absence of a test compound, culturing a second portion of the cells in the presence of a test compound, and detecting a change, for example, an increase or diminution, in a cellular response of the second portion of the cells as compared to the first portion of the cells. The change in cellular response is shown as a measurable change extracellular acidification rate. Moreover, culturing a third portion of the cells in the presence of ZSMF-16 polypeptide and the absence of a test compound can be used as a positive control for the ZSMF-16-responsive cells, and as a control to compare the agonist activity of a test compound with that of the ZSMF-16 polypeptide. Moreover, using the microphysiometer, there is provided a method of identifying antagonists of ZSMF-16 polypeptide, comprising providing cells responsive to a ZSMF-16 polypeptide, culturing a first portion of the cells in the presence of ZSMF-16 and the absence of a test compound, culturing a second portion of the cells in the presence of ZSMF-16 and the presence of a test compound, and detecting a change, for example, an increase or a diminution in a cellular response of the second portion of the cells as compared to the first portion of the cells. The change in cellular response is shown as a measurable change extracellular acidification rate. Antagonists and agonists, for ZSMF-16 polypeptide, can be rapidly identified using this method.

Moreover, ZSMF-16 can be used to identify cells, tissues, or cell lines which respond to a ZSMF-16-stimulated pathway. The microphysiometer, described above, can be used to rapidly identify ligand-responsive cells, such as cells responsive to ZSMF-16 of the present invention. Cells can be cultured in the presence or absence of ZSMF-16 polypeptide. Those cells which elicit a measurable change in extracellular acidification in the presence of ZSMF-16 are responsive to ZSMF-16. Such cell lines, can be used to identify antagonists and agonists of ZSMF-16 polypeptide as described above.

ZSMF-16 polypeptides can also be used to identify inhibitors (antagonists) of its activity. ZSMF-16 antagonists include anti-ZSMF-16 antibodies and soluble ZSMF-16 receptors, as well as other peptidic and non-peptidic agents (including ribozymes). Test compounds are added to the assays disclosed herein to identify compounds that inhibit the activity of ZSMF-16. In addition to those assays disclosed herein, samples can be tested for inhibition of ZSMF-16 activity within a variety of assays designed to measure receptor binding or the stimulation/inhibition of ZSMF-16-dependent cellular responses. For example, ZSMF-16-responsive cell lines can be transfected with a reporter gene construct that is responsive to a ZDMF-7-stimulated cellular pathway. Reporter gene constructs of this type are known in the art, and will generally comprise a ZSMF-16-DNA response element operably linked to a gene encoding an assayable protein, such as luciferase. DNA response elements can include, but are not limited to, cyclic AMP response elements (CRE), hormone response elements (HRE) insulin response element (IRE) (Nasrin et al., Proc. Natl. Acad. Sci. USA 87:5273-7, 1990) and serum response elements (SRE) (Shaw et al. Cell 56: 563-72, 1989). Cyclic AMP response elements are reviewed in Roestler et al., J. Biol. Chem. 263 (19):9063-6; 1988 and Habener, Molec. Endocrinol. 4 (8):1087-94; 1990. Hormone response elements are reviewed in Beato, Cell 56:335-44; 1989. Candidate compounds, solutions, mixtures or extracts are tested for the ability to inhibit the activity of ZSMF-16 on the target cells as evidenced by a decrease in ZSMF-16 stimulation of reporter gene expression. Assays of this type will detect compounds that directly block ZSMF-16 binding to cell-surface receptors, as well as compounds that block processes in the cellular pathway subsequent to receptor-ligand binding. In the alternative, compounds or other samples can be tested for direct blocking of ZSMF-16 binding to receptor using ZSMF-16 tagged with a detectable label (e.g., 1251, biotin, horseradish peroxidase, FITC, and the like). Within assays of this type, the ability of a test sample to inhibit the binding of labeled ZSMF-16 to the receptor is indicative of inhibitory activity, which can be confirmed through secondary assays. Receptors used within binding assays may be cellular receptors or isolated, immobilized receptors.

ZSMF-16 antagonists would find use to modulate or down regulate one or more detrimental biological processes in cells, tissues and/or biological fluids, such as over-responsiveness, unregulated or inappropriate growth, and inflammation or allergic reaction. ZSMF-16 antagonists would have beneficial therapeutic effect in diseases where the inhibition of activation of certain B lymphocytes and/or T cells would be effective. In particular, such diseases would include autoimmune diseases, such as multiple sclerosis, insulin-dependent diabetes and systemic lupus erythematosus. Also, benefit would be derived from using ZSMF-16 antagonists for chronic inflammatory and infective diseases. Antagonists could be used to dampen or inactivate ZSMF-16 during activated immune response.

The activity of semaphorin polypeptides, agonists, antagonists and antibodies of the present invention can be measured, and compounds screened to identify agonists and antagonists, using a variety of assays, such as assays that measure axon guidance and growth. Of particular interest are assays that indicate changes in neuron growth patterns, see for example, Hastings, WIPO Patent Application No:97/29189 and Walter et al., Development 101:685-96, 1987. Assays to measure the effects of semaphorins on neuron growth are well known in the art. For example, the C assay (see for example, Raper and Kapfhammer, Neuron 4:21-9, 1990 and Luo et al., Cell 75:217-27, 1993), can be used to determine collapsing activity semaphorins on growing neurons. Other methods which assess semaphorin induced inhibition of neurite extension or divert such extension are also known, see Goodman, Annu. Rev. Neurosci. 19:341-77, 1996. Conditioned media from cells expressing a semaphorin, such as ZSMF-16, a semaphorin agonist or semaphorin antagonist, or aggregates of such cells, can by placed in a gel matrix near suitable neural cells, such as dorsal root ganglia (DRG) or sympathetic ganglia explants, which have been cocultured with nerve growth factor. Compared to control cells, semaphorin-induced changes in neuron growth can be measured (see for example, Messersmith et al., Neuron 14:949-59, 1995; Puschel et al., Neuron 14:941-8, 1995). Likewise neurite outgrowth can be measured using neuronal cell suspensions grown in the presence of molecules of the present invention see for example, O'Shea et al., Neuron 7:231-7, 1991 and DeFreitas et al., Neuron 15:333-43, 1995. As a semaphorin, these assays described above are preferred assays to measure the biological activity of ZSMF-16 polypeptides, agonists, antagonists and antibodies.

Also available are assay systems that use a ligand-binding receptor (or an antibody, one member of a complement/anti-complement pair) or a binding fragment thereof, and a commercially available biosensor instrument (BIAcore™, Pharmacia Biosensor, Piscataway, N.J.). As used herein, “complement/anti-complement pair” denotes non-identical moieties that form a non-covalently associated, stable pair under appropriate conditions. For instance, biotin and avidin (or streptavidin) are prototypical members of a complement/anti-complement pair. Other exemplary complement/anti-complement pairs include receptor/ligand pairs, antibody/antigen (or hapten or epitope) pairs, sense/antisense polynucleotide pairs, and the like. Where subsequent dissociation of the complement/anti-complement pair is desirable, the complement/anti-complement pair preferably has a binding affinity of <10⁹ M⁻¹. Such receptor, antibody, member of a complement/anti-complement pair or fragment is immobilized onto the surface of a receptor chip. Use of this instrument is disclosed by Karlsson, J. Immunol. Methods 145:229-40, 1991 and Cunningham and Wells, J. Mol. Biol. 234:554-63, 1993. A receptor, antibody, member or fragment is covalently attached, using amine or sulfhydryl chemistry, to dextran fibers that are attached to gold film within the flow cell. A test sample is passed through the cell. If a ligand, epitope, or opposite member of the complement/anti-complement pair is present in the sample, it will bind to the immobilized receptor, antibody or member, respectively, causing a change in the refractive index of the medium, which is detected as a change in surface plasmon resonance of the gold film. This system allows the determination of on- and off-rates, from which binding affinity can be calculated, and assessment of stoichiometry of binding. Ligand-binding receptor polypeptides can also be used within other assay systems known in the art. Such systems include Scatchard analysis for determination of binding affinity (see, Scatchard, Ann. NY Acad. Sci. 51: 660-72, 1949) and calorimetric assays (Cunningham et al., Science 253:545-8, 1991; Cunningham et al., Science 245:821-5, 1991).

An in vivo approach for assaying proteins of the present invention involves viral delivery systems. Exemplary viruses for this purpose include adenovirus, herpesvirus, retroviruses, vaccinia virus, and adeno-associated virus (AAV). Adenovirus, a double-stranded DNA virus, is currently the best studied gene transfer vector for delivery of heterologous nucleic acid (for review, see T. C. Becker et al., Meth. Cell Biol. 43:161-89, 1994; and J. T. Douglas and D. T. Curiel, Science & Medicine 4:44-53, 1997). The adenovirus system offers several advantages: (i) adenovirus can accommodate relatively large DNA inserts; (ii) can be grown to high-titer; (iii) infect a broad range of mammalian cell types; and (iv) can be used with many different promoters including ubiquitous, tissue specific, and regulatable promoters. Also, because adenoviruses are stable in the bloodstream, they can be administered by intravenous injection.

Using adenovirus vectors where portions of the adenovirus genome are deleted, inserts are incorporated into the viral DNA by direct ligation or by homologous recombination with a co-transfected plasmid. In an exemplary system, the essential El gene has been deleted from the viral vector, and the virus will not replicate unless the E1 gene is provided by the host cell (the human 293 cell line is exemplary). When intravenously administered to intact animals, adenovirus primarily targets the liver. If the adenoviral delivery system has an E1 gene deletion, the virus cannot replicate in the host cells. However, the host's tissue (e.g., liver) will express and process (and, if a secretory signal sequence is present, secrete) the heterologous protein. Secreted proteins will enter the circulation in the highly vascularized liver, and effects on the infected animal can be determined.

Moreover, adenoviral vectors containing various deletions of viral genes can be used in an attempt to reduce or eliminate immune responses to the vector. Such adenoviruses are E1 deleted, and in addition contain deletions of E2A or E4 (Lusky, M. et al., J. Virol. 72:2022-2032, 1998; Raper, S. E. et al., Human Gene Therapy 9:671-679, 1998). In addition, deletion of E2b is reported to reduce immune responses (Amalfitano, A. et al., J. Virol. 72:926-933, 1998). Moreover, by deleting the entire adenovirus genome, very large inserts of heterologous DNA can be accommodated. Generation of so called “gutless” adenoviruses where all viral genes are deleted are particularly advantageous for insertion of large inserts of heterologous DNA. For review, see Yeh, P. and Perricaudet, M., FASEB J. 11:615-623, 1997.

The adenovirus system can also be used for protein production in vitro. By culturing adenovirus-infected non-293 cells under conditions where the cells are not rapidly dividing, the cells can produce proteins for extended periods of time. For instance, BHK cells are grown to confluence in cell factories, then exposed to the adenoviral vector encoding the secreted protein of interest. The cells are then grown under serum-free conditions, which allows infected cells to survive for several weeks without significant cell division. Alternatively, adenovirus vector infected 293 cells can be grown as adherent cells or in suspension culture at relatively high cell density to produce significant amounts of protein (See Garnier et al., Cytotechnol. 15:145-55, 1994). With either protocol, an expressed, secreted heterologous protein can be repeatedly isolated from the cell culture supernatant, lysate, or membrane fractions depending on the disposition of the expressed protein in the cell. Within the infected 293 cell production protocol, non-secreted proteins may also be effectively obtained.

Differentiation is a progressive and dynamic process, beginning with pluripotent stem cells and ending with terminally differentiated cells. Pluripotent stem cells that can regenerate without commitment to a lineage express a set of differentiation markers that are lost when commitment to a cell lineage is made. Progenitor cells express a set of differentiation markers that may or may not continue to be expressed as the cells progress down the cell lineage pathway toward maturation. Differentiation markers that are expressed exclusively by mature cells are usually functional properties such as cell products, enzymes to produce cell products, and receptors. The stage of a cell population's differentiation is monitored by identification of markers present in the cell population. Myocytes, osteoblasts, adipocytes, chrondrocytes, fibroblasts and reticular cells are believed to originate from a common mesenchymal stem cell (Owen et al., Ciba Fdn. Symp. 136:42-46, 1988). Markers for mesenchymal stem cells have not been well identified (Owen et al., J. of Cell Sci. 87:731-738, 1987), so identification is usually made at the progenitor and mature cell stages. The novel polypeptides of the present invention may be useful for studies to isolate mesenchymal stem cells and myocyte or other progenitor cells, both in vivo and ex vivo.

There is evidence to suggest that factors that stimulate specific cell types down a pathway towards terminal differentiation or dedifferentiation affect the entire cell population originating from a common precursor or stem cell. Thus, the present invention includes stimulating or inhibiting the proliferation of myocytes, smooth muscle cells, osteoblasts, adipocytes, chrondrocytes, neuronal and endothelial cells. Molecules of the present invention for example, may while stimulating proliferation or differentiation of cardiac myocytes, inhibit proliferation or differentiation of adipocytes, by virtue of the affect on their common precursor/stem cells. Thus molecules of the present invention may have use in inhibiting chondrosarcomas, atherosclerosis, restenosis and obesity.

Assays measuring differentiation include, for example, measuring cell markers associated with stage-specific expression of a tissue, enzymatic activity, functional activity or morphological changes (Watt, FASEB 5:281-284, 1991; Francis, Differentiation 57:63-75, 1994; Raes, Adv. Anim. Cell Biol. Technol. Bioprocesses, 161-171, 1989; all incorporated herein by reference). Alternatively, ZSMF-16 polypeptide itself can serve as an additional cell-surface or secreted marker associated with stage-specific expression of a tissue. As such, direct measurement of ZSMF-16 polypeptide, or its loss of expression in a tissue as it differentiates, can serve as a marker for differentiation of tissues.

Differentiation is a progressive and dynamic process, beginning with pluripotent stem cells and ending with terminally differentiated cells. Pluripotent stem cells that can regenerate without commitment to a lineage express a set of differentiation markers that are lost when commitment to a cell lineage is made. Progenitor cells express a set of differentiation markers that may or may not continue to be expressed as the cells progress down the cell lineage pathway toward maturation. Differentiation markers that are expressed exclusively by mature cells are usually functional properties such as cell products, enzymes to produce cell products, and receptors. The stage of a cell population's differentiation is monitored by identification of markers present in the cell population. The novel polypeptides of the present invention may be useful for studies to isolate stem cells and neuronal or other progenitor cells, both in vivo and ex vivo.

There is evidence to suggest that factors that stimulate specific cell types down a pathway towards terminal differentiation or dedifferentiation affect the entire cell population originating from a common precursor or stem cell. Assays measuring differentiation include, for example, measuring cell markers associated with stage-specific expression of a tissue, enzymatic activity, functional activity or morphological changes (Watt, FASEB, 5:281-284, 1991; Francis, Differentiation 57:63-75, 1994; Raes, Adv. Anim. Cell Biol. Technol. Bioprocesses, 161-171, 1989; all incorporated herein by reference). Alternatively, ZSMF-16 polypeptide itself can serve as an additional cell-surface or secreted marker associated with stage-specific expression of a tissue, such as testis tissue. As such, direct measurement of ZSMF-16 polypeptide, or its loss of expression in a tissue as it differentiates, can serve as a marker for differentiation of tissues.

Similarly, direct measurement of ZSMF-16 polypeptide, or its loss of expression in a tissue can be determined in a tissue or cells as they undergo tumor or disease progression. Increases in invasiveness and motility of cells, or the gain or loss of expression of ZSMF-16 in a pre-cancerous or cancerous condition, in comparison to normal tissue, can serve as a diagnostic for transformation, invasion and metastasis in tumor progression. As such, knowledge of a tumor's stage of progression or metastasis will aid the physician in choosing the most proper therapy, or aggressiveness of treatment, for a given individual cancer patient. Methods of measuring gain and loss of expression (of either MRNA or protein) are well known in the art and described herein and can be applied to ZSMF-16 expression. For example, appearance or disappearance of polypeptides that regulate cell motility can be used to aid diagnosis and prognosis of prostate cancer (Banyard, J. and Zetter, B. R., Cancer and Metast. Rev. 17:449-458, 1999). As an effector of cell motility, or as a testis-specific marker, ZSMF-16 gain or loss of expression may serve as a diagnostic for mammary tumor tissue, or breast tumor and diseased breast, and other cancers. Moreover, analogous to the prostate specific antigen (PSA), as a naturally-expressed testicular marker, increased levels of ZSMF-16 polypeptides, or anti-ZSMF-16 antibodies in a patient, relative to a normal control can be indicative of breast, liver, small intestine, bone, brain diseases, such as breast, liver, intestinal, bone or brain cancer (See, e.g., Mulders, TMT, et al., Eur. J. Surgical Oncol. 16:37-41, 1990). Moreover, as ZSMF-16 expression appears to be restricted to specific human tissues, lack of ZSMF-16 expression in those tissues or strong ZSMF-16 expression in tissues where ZSMF-16 is not normally expressed, would serve as a diagnostic of an abnormality in the cell or tissue type, of invasion or metastasis of cancerous testicular tissues into non-testicular tissue, and could aid a physician in directing further testing or investigation, or aid in directing therapy.

In addition, as ZSMF-16 is as breast, liver, intestinal, bone, and brain-specific, polynucleotide probes, anti-ZSMF-16 antibodies, and detection the presence of ZSMF-16 polypeptides in tissue can be used to assess whether these tissues are present, for example, after surgery involving the excision of a diseased or cancerous breast, liver, intestinal, bone or brain tissue. As such, the polynucleotides, polypeptides, and antibodies of the present invention can be used as an aid to determine whether all such tissue is excised after surgery, for example, after surgery for cancer. In such instances, it is especially important to remove all potentially diseased tissue to maximize recovery from the cancer, and to minimize recurrence. Preferred embodiments include fluorescent, radiolabeled, or calorimetrically labeled anti-ZSMF-16 antibodies and ZSMF-16 polypeptide binding partners, that can be used histologically or in situ.

Similarly, direct measurement of ZSMF-16 polypeptide, or its loss of expression in a tissue can be determined in a tissue or cells as they undergo tumor progression. Increases in invasiveness and motility of cells, or the gain or loss of expression of ZSMF-16 in a pre-cancerous or cancerous condition, in comparison to normal tissue, can serve as a diagnostic for transformation, invasion and metastasis in tumor progression. As such, knowledge of a tumor's stage of progression or metastasis will aid the physician in choosing the most proper therapy, or aggressiveness of treatment, for a given individual cancer patient. Methods of measuring gain and loss of expression (of either mRNA or protein) are well known in the art and described herein and can be applied to ZSMF-16 expression. For example, appearance or disappearance of polypeptides that regulate cell motility can be used to aid diagnosis and prognosis of prostate cancer (Banyard, J. and Zetter, B. R., Cancer and Metast. Rev. 17:449-458, 1999). As an effector of cell motility, ZSMF-16 gain or loss of expression may serve as a diagnostic for neuronal and other cancers.

Moreover, the activity and effect of ZSMF-16 on tumor progression and metastasis can be measured in vivo. Several syngeneic mouse models have been developed to study the influence of polypeptides, compounds or other treatments on tumor progression. In these models, tumor cells passaged in culture are implanted into mice of the same strain as the tumor donor. The cells will develop into tumors having similar characteristics in the recipient mice, and metastasis will also occur in some of the models. Appropriate tumor models for our studies include the Lewis lung carcinoma (ATCC No. CRL-1642) and B16 melanoma (ATCC No. CRL-6323), amongst others. These are both commonly used tumor lines, syngeneic to the C57BL6 mouse, that are readily cultured and manipulated in vitro. Tumors resulting from implantation of either of these cell lines are capable of metastasis to the lung in C57BL6 mice. The Lewis lung carcinoma model has recently been used in mice to identify an inhibitor of angiogenesis (O'Reilly M S, et al. Cell 79: 315-328,1994). C57BL6/J mice are treated with an experimental agent either through daily injection of recombinant protein, agonist or antagonist or a one time injection of recombinant adenovirus. Three days following this treatment, 10⁵ to 10⁶ cells are implanted under the dorsal skin. Alternatively, the cells themselves may be infected with recombinant adenovirus, such as one expressing ZSMF-16, before implantation so that the protein is synthesized at the tumor site or intracellularly, rather than systemically. The mice normally develop visible tumors within 5 days. The tumors are allowed to grow for a period of up to 3 weeks, during which time they may reach a size of 1500 - 1800 mm³ in the control treated group. Tumor size and body weight are carefully monitored throughout the experiment. At the time of sacrifice, the tumor is removed and weighed along with the lungs and the liver. The lung weight has been shown to correlate well with metastatic tumor burden. As an additional measure, lung surface metastases are counted. The resected tumor, lungs and liver are prepared for histopathological examination, immunohistochemistry, and in situ hybridization, using methods known in the art and described herein. The influence of the expressed polypeptide in question, e.g., ZSMF-16, on the ability of the tumor to recruit vasculature and undergo metastasis can thus be assessed. In addition, aside from using adenovirus, the implanted cells can be transiently transfected with ZSMF-16. Use of stable ZSMF-16 transfectants as well as use of induceable promoters to activate ZSMF-16 expression in vivo are known in the art and can be used in this system to assess ZSMF-16 induction of metastasis. Moreover, purified ZSMF-16 or ZSMF-16 conditioned media can be directly injected in to this mouse model, and hence be used in this system. For general reference see, O'Reilly M S, et al. Cell 79:315-328, 1994; and Rusciano D, et al. Murine Models of Liver Metastasis. Invasion Metastasis 14:349-361, 1995.

ZSMF-16 polypeptides can also be used to prepare antibodies that bind to ZSMF-16 epitopes, peptides or polypeptides. The ZSMF-16 polypeptide or a fragment thereof serves as an antigen (immunogen) to inoculate an animal and elicit an immune response. One of skill in the art would recognize that antigenic, epitope-bearing polypeptides contain a sequence of at least 6, preferably at least 9, and more preferably at least 15 to about 30 contiguous amino acid residues of a ZSMF-16 polypeptide (e.g., SEQ ID NO:2). Polypeptides comprising a larger portion of a ZSMF-16 polypeptide, i.e., from 30 to 10 residues up to the entire length of the amino acid sequence are included. Antigens or immunogenic epitopes can also include attached tags, adjuvants and carriers, as described herein. Suitable antigens include the ZSMF-16 polypeptide encoded by SEQ ID NO:2 from amino acid number 23 (Gly) to amino acid number 779 (Thr) or a contiguous 9 to 757 amino acid fragment thereof. More preferably suitable antigens include the ZSMF-16 polypeptide encoded by SEQ ID NO:2 from amino acid number 39 (Gly) to amino acid number 57 (Tyr) or a contiguous 9 to 19 amino acid fragment thereof. Other suitable antigens include the N-terminal region, semaphorin domain, middle domain, Ig-like domain, C-terminal domain, basic domain and other domains and motifs as described herein. Preferred peptides to use as antigens are hydrophilic peptides such as those predicted by one of skill in the art from a hydrophobicity plot (See Figure). ZSMF-16 hydrophilic peptides include peptides comprising amino acid sequences selected from the group consisting of: (1) amino acid number 82 (Asp) to amino acid number 87 (Arg) of SEQ ID NO:2; (2) amino acid number 234 (Asp) to amino acid number 239 (Lys) of SEQ ID NO:2; (3) amino acid number 395 (Lys) to amino acid number 400 (Glu) of SEQ ID NO:2; (4) amino acid number 553 (Lys) to amino acid number 558 (Arg) of SEQ ID NO:2; and (5) amino acid number 683 (Glu) to amino acid number 688 (Glu) of SEQ ID NO:2. Moreover, ZSMF-16 antigenic epitopes as predicted by a Jameson-Wolf plot, e.g., using DNASTAR Protean program (DNASTAR, Inc., Madison, Wis.) serve as preferred antigens. Such preferred antigens can be readily determined by one of skill in the art. Other preferred antigens include residues 39 (Gly) to 57 (Tyr) of SEQ ID NO:2; and residues 107 (Asp) to 114 (Ala) of SEQ ID NO:2, plus or minus up to 2 amino acids of SEQ ID NO:2 on either or both ends (e.g., 105-114, 109-114, 107-116, 107-112, 105-112, 105-114, 105-116, 109-116 of SEQ ID NO:2). Antibodies from an immune response generated by inoculation of an animal with these antigens can be isolated and purified as described herein. Methods for preparing and isolating polyclonal and monoclonal antibodies are well known in the art. See, for example, Current Protocols in Immunology, Cooligan, et al. (eds.), National Institutes of Health, John Wiley and Sons, Inc., 1995; Sambrook et al., Molecular Cloning: A Laboratory Manual, Second Edition, Cold Spring Harbor, N.Y., 1989; and Hurrell, J. G. R., Ed., Monoclonal Hybridoma Antibodies: Techniques and Applications, CRC Press, Inc., Boca Raton, Fla., 1982.

As would be evident to one of ordinary skill in the art, polyclonal antibodies can be generated from inoculating a variety of warm-blooded animals such as horses, cows, goats, sheep, dogs, chickens, rabbits, mice, and rats with a ZSMF-16 polypeptide or a fragment thereof. The immunogenicity of a ZSMF-16 polypeptide may be increased through the use of an adjuvant, such as alum (aluminum hydroxide) or Freund's complete or incomplete adjuvant. Polypeptides useful for immunization also include fusion polypeptides, such as fusions of ZSMF-16 or a portion thereof with an immunoglobulin polypeptide or with maltose binding protein. The polypeptide immunogen may be a full-length molecule or a portion thereof. If the polypeptide portion is “hapten-like”, such portion may be advantageously joined or linked to a macromolecular carrier (such as keyhole limpet hemocyanin (KLH), bovine serum albumin (BSA) or tetanus toxoid) for immunization.

As used herein, the term “antibodies” includes polyclonal antibodies, affinity-purified polyclonal antibodies, monoclonal antibodies, and antigen-binding fragments, such as F(ab′)₂ and Fab proteolytic fragments. Genetically engineered intact antibodies or fragments, such as chimeric antibodies, Fv fragments, single chain antibodies and the like, as well as synthetic antigen-binding peptides and polypeptides, are also included. Non-human antibodies may be humanized by grafting non-human CDRs onto human framework and constant regions, or by incorporating the entire non-human variable domains (optionally “cloaking” them with a human-like surface by replacement of exposed residues, wherein the result is a “veneered” antibody). In some instances, humanized antibodies may retain non-human residues within the human variable region framework domains to enhance proper binding characteristics. Through humanizing antibodies, biological half-life may be increased, and the potential for adverse immune reactions upon administration to humans is reduced. Moreover, human antibodies can be produced in transgenic, non-human animals that have been engineered to contain human immunoglobulin genes as disclosed in WIPO Publication WO 98/24893. It is preferred that the endogenous immunoglobulin genes in these animals be inactivated or eliminated, such as by homologous recombination.

Antibodies are considered to be specifically binding if: 1) they exhibit a threshold level of binding activity, and 2) they do not significantly cross-react with related polypeptide molecules. A threshold level of binding is determined if anti-ZSMF-16 antibodies herein bind to a ZSMF-16 polypeptide, peptide or epitope with an affinity at least 10-fold greater than the binding affinity to control (non-ZSMF-16) polypeptide. It is preferred that the antibodies exhibit a binding affinity (Kₐ) of 10⁶ M⁻¹ or greater, preferably 10⁷ M⁻¹ or greater, more preferably 10⁸ M⁻¹ or greater, and most preferably 10⁹ M⁻¹ or greater. The binding affinity of an antibody can be readily determined by one of ordinary skill in the art, for example, by Scatchard analysis (Scatchard, G., Ann. NY Acad. Sci. 51: 660-672, 1949).

Whether anti-ZSMF-16 antibodies do not significantly cross-react with related polypeptide molecules is shown, for example, by the antibody detecting ZSMF-16 polypeptide but not known related polypeptides using a standard Western blot analysis (Ausubel et al., ibid.). Examples of known related polypeptides are those disclosed in the prior art, such as known orthologs, and paralogs, and similar known members of a protein family, Screening can also be done using non-human ZSMF-16, and ZSMF-16 mutant polypeptides. Moreover, antibodies can be “screened against” known related polypeptides, to isolate a population that specifically binds to the ZSMF-16 polypeptides. For example, antibodies raised to ZSMF-16 are adsorbed to related polypeptides adhered to insoluble matrix; antibodies specific to ZSMF-16 will flow through the matrix under the proper buffer conditions. Screening allows isolation of polyclonal and monoclonal antibodies non-crossreactive to known closely related polypeptides (Antibodies: A Laboratory Manual, Harlow and Lane (eds.), Cold Spring Harbor Laboratory Press, 1988; Current Protocols in Immunology, Cooligan, et al. (eds.), National Institutes of Health, John Wiley and Sons, Inc., 1995). Screening and isolation of specific antibodies is well known in the art. See, Fundamental Immunology, Paul (eds.), Raven Press, 1993; Getzoff et al., Adv. in Immuno. 43: 1-98, 1988; Monoclonal Antibodies: Principles and Practice, Goding, J. W. (eds.), Academic Press Ltd., 1996; Benjamin et al., Ann. Rev. Immunol. 2: 67-101, 1984. Specifically binding anti-ZSMF-16 antibodies can be detected by a number of methods in the art, and disclosed below.

A variety of assays known to those skilled in the art can be utilized to detect antibodies which bind to ZSMF-16 proteins or polypeptides. Exemplary assays are described in detail in Antibodies: A Laboratory Manual, Harlow and Lane (Eds.), Cold Spring Harbor Laboratory Press, 1988. Representative examples of such assays include: concurrent immunoelectrophoresis, radioimmunoassay, radioimmuno-precipitation, enzyme-linked immunosorbent assay (ELISA), dot blot or Western blot assay, inhibition or competition assay, and sandwich assay. In addition, antibodies can be screened for binding to wild-type versus mutant ZSMF-16 protein or polypeptide.

Alternative techniques for generating or selecting antibodies useful herein include in vitro exposure of lymphocytes to ZSMF-16 protein or peptide, and selection of antibody display libraries in phage or similar vectors (for instance, through use of immobilized or labeled ZSMF-16 protein or peptide). Genes encoding polypeptides having potential ZSMF-16 polypeptide binding domains can be obtained by screening random peptide libraries displayed on phage (phage display) or on bacteria, such as E. coli. Nucleotide sequences encoding the polypeptides can be obtained in a number of ways, such as through random mutagenesis and random polynucleotide synthesis. These random peptide display libraries can be used to screen for peptides which interact with a known target which can be a protein or polypeptide, such as a ligand or receptor, a biological or synthetic macromolecule, or organic or inorganic substances. Techniques for creating and screening such random peptide display libraries are known in the art (Ladner et al., U.S. Pat. No. 5,223,409; Ladner et al., U.S. Pat. No. 4,946,778; Ladner et al., U.S. Pat. No. 5,403,484 and Ladner et al., U.S. Pat. No. 5,571,698) and random peptide display libraries and kits for screening such libraries are available commercially, for instance from Clontech (Palo Alto, Calif.), Invitrogen Inc. (San Diego, Calif.), New England Biolabs, Inc. (Beverly, Mass.) and Pharmacia LKB Biotechnology Inc. (Piscataway, N.J.). Random peptide display libraries can be screened using the ZSMF-16 sequences disclosed herein to identify proteins which bind to ZSMF-16. These “binding polypeptides” which interact with ZSMF-16 polypeptides can be used for tagging cells; for isolating homolog polypeptides by affinity purification; they can be directly or indirectly conjugated to drugs, toxins, radionuclides and the like. These binding polypeptides can also be used in analytical methods such as for screening expression libraries and neutralizing activity, e.g., for blocking interaction between ligand and receptor, or viral binding to a receptor. The binding polypeptides can also be used for diagnostic assays for determining circulating levels of ZSMF-16 polypeptides; for detecting or quantitating soluble ZSMF-16 polypeptides as marker of underlying pathology or disease. These binding polypeptides can also act as ZSMF-16 “antagonists” to block ZSMF-16 binding and signal transduction in vitro and in vivo. These anti-ZSMF-16 binding polypeptides would be useful for inhibiting ZSMF-16 activity or protein-binding.

Antibodies to ZSMF-16 may be used for tagging cells that express ZSMF-16; for isolating ZSMF-16 by affinity purification; for diagnostic assays for determining circulating levels of ZSMF-16 polypeptides; for detecting or quantitating soluble ZSMF-16 as marker of underlying pathology or disease; in analytical methods employing FACS; for screening expression libraries; for generating anti-idiotypic antibodies; and as neutralizing antibodies or as antagonists to block ZSMF-16 activity in vitro and in vivo. Suitable direct tags or labels include radionuclides, enzymes, substrates, cofactors, inhibitors, fluorescent markers, chemiluminescent markers, magnetic particles and the like; indirect tags or labels may feature use of biotin-avidin or other complement/anti-complement pairs as intermediates. Antibodies herein may also be directly or indirectly conjugated to drugs, toxins, radionuclides and the like, and these conjugates used for in vivo diagnostic or therapeutic applications. Moreover, antibodies to ZSMF-16 or fragments thereof may be used in vitro to detect denatured ZSMF-16 or fragments thereof in assays, for example, Western Blots or other assays known in the art.

Genes encoding polypeptides having potential ZSMF-16 polypeptide binding domains can be obtained by screening random peptide libraries displayed on phage (phage display) or on bacteria, such as E. coli. Nucleotide sequences encoding the binding polypeptides can be obtained in a number of ways, such as through random mutagenesis and random polynucleotide synthesis. These random peptide display libraries can be used to screen for peptides which interact with a known target which can be a protein or polypeptide, such as a ligand or receptor, a biological or synthetic macromolecule, or organic or inorganic substances. Techniques for creating and screening such random peptide display libraries are known in the art (Ladner et al., U.S. Pat. No. 5,223,409; Ladner et al., U.S. Pat. No. 4,946,778; Ladner et al., U.S. Pat. No. 5,403,484 and Ladner et al., U.S. Pat. No. 5,571,698) and random peptide display libraries and kits for screening such libraries are available commercially, for instance from Clontech (Palo Alto, Calif.), Invitrogen Inc. (San Diego, Calif.), New England Biolabs, Inc. (Beverly, Mass.) and Pharmacia LKB Biotechnology Inc. (Piscataway, N.J.). Random peptide display libraries can be screened using the ZSMF-16 sequences disclosed herein to identify proteins which bind to ZSMF-16. These “binding polypeptides” which interact with ZSMF-16 polypeptides can be used for tagging cells; for isolating homolog polypeptides by affinity purification; they can be directly or indirectly conjugated to drugs, toxins, radionuclides and the like. These binding polypeptides can also be used in analytical methods such as for screening expression libraries and neutralizing activity. The binding polypeptides can also be used for diagnostic assays for determining circulating levels of polypeptides; for detecting or quantitating soluble polypeptides as marker of underlying pathology or disease. These binding polypeptides can also act as ZSMF-16 “antagonists” to block ZSMF-16 binding and signal transduction in vitro and in vivo. These anti-ZSMF-16 binding polypeptides would be useful for inhibiting ZSMF-16 binding.

ZSMF-16 polypeptides and polynucleotides may be used within diagnostic systems. Antibodies or other agents that specifically bind to ZSMF-16 may be used to detect the presence of circulating ligand or receptor polypeptides. Such detection methods are well known in the art and include, for example, enzyme-linked immunosorbent assay (ELISA) and radioimmunoassay. Immunohistochemically labeled ZSMF-16 antibodies can be used to detect ZSMF-16 receptor and/or ligands in tissue samples and identify ZSMF- 16 receptors. ZSMF-16 levels can also be monitored by such methods as RT-PCR, where ZSMF-16 mRNA can be detected and quantified. The information derived from such detection methods would provide insight into the significance of ZSMF-16 polypeptides in various diseases and biological processes, and as such would serve as diagnostic tools for diseases for which altered levels of ZSMF-16 are significant.

Nucleic acid molecules disclosed herein can be used to detect the expression of a ZSMF-16 gene in a biological sample. Such probe molecules include double-stranded nucleic acid molecules comprising the nucleotide sequences of SEQ ID NO:1 or SEQ ID NO:3, or fragments thereof, as well as single-stranded nucleic acid molecules having the complement of the nucleotide sequences of SEQ ID NO:1 or SEQ ID NO:3, or a fragment thereof. Probe molecules may be DNA, RNA, oligonucleotides, and the like. For example, suitable probes include nucleic acid molecules that bind with a portion of a ZSMF-16 domain or motif disclosed herein, such as the ZSMF-16 semaphorin domain. Other probes include those to the N-terminal region, semaphorin domain, middle domain, Ig-like domain, C-terminal domain, basic domain and other domains and motifs as described herein. In a basic assay, a single-stranded probe molecule is incubated with RNA, isolated from a biological sample, under conditions of temperature and ionic strength that promote base pairing between the probe and target ZSMF-16 RNA species. After separating unbound probe from hybridized molecules, the level and length of the hybrid is detected. Well-established hybridization methods of RNA detection include northern analysis and dot/slot blot hybridization, see, for example, Ausubel ibid. and Wu et al. (eds.), “Analysis of Gene Expression at the RNA Level,” in Methods in Gene Biotechnology, pages 225-239 (CRC Press, Inc. 1997), and methods described herein. Nucleic acid probes can be detectably labeled with radioisotopes such as ³²P or ³⁵S. Alternatively, ZSMF-16 RNA can be detected with a nonradioactive hybridization method (see, for example, Isaac (ed.), Protocols for Nucleic Acid Analysis by Nonradioactive Probes, Humana Press, Inc., 1993). Typically, nonradioactive detection is achieved by enzymatic conversion of chromogenic or chemiluminescent substrates. Illustrative non-radioactive moieties include biotin, fluorescein, and digoxigenin.

ZSMF-16 oligonucleotide probes are also useful for in vivo diagnosis. For example, ¹⁸F-labeled oligonucleotides can be administered to a subject and visualized by positron emission tomography (Tavitian et al., Nature Medicine 4:467, 1998). Moreover, numerous diagnostic procedures take advantage of the polymerase chain reaction (PCR) to increase sensitivity of detection methods. Standard techniques for performing PCR are well-known (see, generally, Mathew (ed.), Protocols in Human Molecular Genetics (Humana Press, Inc. 1991), White (ed.), PCR Protocols: Current Methods and Applications (Humana Press, Inc. 1993), Cotter (ed.), Molecular Diagnosis of Cancer (Humana Press, Inc. 1996), Hanausek and Walaszek (eds.), Tumor Marker Protocols (Humana Press, Inc. 1998), Lo (ed.), Clinical Applications of PCR (Humana Press, Inc. 1998), and Meltzer (ed.), PCR in Bioanalysis (Humana Press, Inc. 1998)). PCR primers can be designed to amplify a sequence encoding a full-length or partial ZSMF-16 polynucleotide, or a particular ZSMF-16 domain or motif, such as the ZSMF-16 semaphorin domain as disclosed herein.

One variation of PCR for diagnostic assays is reverse transcriptase-PCR (RT-PCR). In the RT-PCR technique, RNA is isolated from a biological sample, reverse transcribed to cDNA, and the cDNA is incubated with ZSMF-16 primers (see, for example, Wu et al. (eds.), “Rapid Isolation of Specific cDNAs or Genes by PCR,” in Methods in Gene Biotechnology, CRC Press, Inc., pages 15-28, 1997). PCR is then performed and the products are analyzed using standard techniques. For example, RNA is isolated from biological sample using, for example, the guanidinium-isothiocyanate cell lysis procedure described herein. Alternatively, a solid-phase technique can be used to isolate mRNA from a cell lysate. A reverse transcription reaction can be primed with the isolated RNA using random oligonucleotides, short homopolymers of dT, or ZSMF-16 anti-sense oligomers. Oligo-dT primers offer the advantage that various mRNA nucleotide sequences are amplified that can provide control target sequences. ZSMF-16 sequences are amplified by the polymerase chain reaction using two flanking oligonucleotide primers. PCR amplification products can be detected using a variety of approaches. For example, PCR products can be fractionated by gel electrophoresis, and visualized by ethidium bromide staining. Alternatively, fractionated PCR products can be transferred to a membrane, hybridized with a detectably-labeled ZSMF-16 probe, and examined by autoradiography. Additional alternative approaches include the use of digoxigenin-labeled deoxyribonucleic acid triphosphates to provide chemiluminescence detection, and the C-TRAK calorimetric assay. Another approach is to use real time quantitative PCR (Perkin-Elmer Cetus, Norwalk, Conn.). A fluorogenic probe, consisting of an oligonucleotide with both a reporter and a quencher dye attached, anneals specifically between the forward and reverse primers. Using the 5′ endonuclease activity of Taq DNA polymerase, the reporter dye is separated from the quencher dye and a sequence-specific signal is generated and increases as amplification increases. The fluorescence intensity can be continuously monitored and quantified during the PCR reaction.

Another approach for detection of ZSMF-16 expression is cycling probe technology (CPT), in which a single-stranded DNA target binds with an excess of DNA-RNA-DNA chimeric probe to form a complex, the RNA portion is cleaved with RNase H, and the presence of cleaved chimeric probe is detected (see, for example, Beggs et al., J. Clin. Microbiol. 34:2985, 1996 and Bekkaoui et al., Biotechniques 20:240, 1996). Alternative methods for detection of ZSMF-16 sequences can utilize approaches such as nucleic acid sequence-based amplification (NASBA), cooperative amplification of templates by cross-hybridization (CATCH), and the ligase chain reaction (LCR) (see, for example, Marshall et al., U.S. Pat. No. 5,686,272 (1997), Dyer et al., J. Virol. Methods 60:161, 1996; Ehricht et al., Eur. J. Biochem. 243:358, 1997 and Chadwick et al., J. Virol. Methods 70:59, 1998). Other standard methods are known to those of skill in the art.

ZSMF-16 probes and primers can also be used to detect and to localize ZSMF-16 gene expression in tissue samples. Methods for such in situ hybridization are well-known to those of skill in the art (see, for example, Choo (ed.), In Situ Hybridization Protocols, Humana Press, Inc., 1994; Wu et al. (eds.), “Analysis of Cellular DNA or Abundance of mRNA by Radioactive In Situ Hybridization (RISH),” in Methods in Gene Biotechnology, CRC Press, Inc., pages 259-278, 1997 and Wu et al. (eds.), “Localization of DNA or Abundance of mRNA by Fluorescence In Situ Hybridization (RISH),” in Methods in Gene Biotechnology, CRC Press, Inc., pages 279-289, 1997). Various additional diagnostic approaches are well-known to those of skill in the art (see, for example, Mathew (ed.), Protocols in Human Molecular Genetics Humana Press, Inc., 1991; Coleman and Tsongalis, Molecular Diagnostics, Humana Press, Inc., 1996 and Elles, Molecular Diagnosis of Genetic Diseases, Humana Press, Inc., 1996).

The ZSMF-16 polynucleotides and/or polypeptides disclosed herein can be useful as therapeutics, wherein ZSMF-16 agonists and antagonists could modulate one or more biological processes in cells, tissues and/or biological fluids. ZSMF-16 antagonists provided by the invention, bind to ZSMF-16 polypeptides or, alternatively, to a receptor to which ZSMF-16 polypeptides bind, thereby inhibiting or eliminating the function of ZSMF-16. Such ZSMF-16 antagonists would include antibodies; oligonucleotides which bind either to the ZSMF-16 polypeptide or to its ligand; natural or synthetic analogs of ZSMF-16 ligands which retain the ability to bind the receptor but do not result in either ligand or receptor signaling. Such analogs could be peptides or peptide-like compounds. Natural or synthetic small molecules which bind to ZSMF-16 polypeptides and prevent signaling are also contemplated as antagonists. As such, ZSMF-16 antagonists would be useful as therapeutics for treating certain disorders where blocking signal from either a ZSMF-16 receptor or ligand would be beneficial.

The invention also provides nucleic acid-based therapeutic treatment. If a mammal lacks or has a mutated ZSMF-16 gene, the ZSMF-16 gene can be introduced into the cells of the mammal. Using such methods, cells altered to express the nerve growth factor neurotrophin-3 (NT-3) were grafted to a rat model for spinal injury and stimulated axon regrowth at the lesion site and the rats thus treated recovered some ability to walk (Grill et al., J. Neuroscience 17:5560-72, 1997). In one embodiment, a gene encoding a ZSMF-16 polypeptide is introduced in vivo in a viral vector. Such vectors include an attenuated or defective DNA virus, such as but not limited to herpes simplex virus (HSV), papillomavirus, Epstein Barr virus (EBV), adenovirus, adeno-associated virus (AAV), and the like. Defective viruses, which entirely or almost entirely lack viral genes, are preferred. A defective virus is not infective after introduction into a cell. Use of defective viral vectors allows for administration to cells in a specific, localized area, without concern that the vector can infect other cells. Examples of particular vectors include, but are not limited to, a defective herpes virus 1 (HSV1) vector (Kaplitt et al., Molec. Cell. Neurosci. 2:320-30, 1991), an attenuated adenovirus vector, such as the vector described by Stratford-Perricaudet et al. (J. Clin. Invest. 90:626-30, 1992), and a defective adeno-associated virus vector (Samulski et al., J. Virol. 61:3096-101, 1987; Samulski et al., J. Virol. 63:3822-8, 1989).

In another embodiment, the gene can be introduced in a retroviral vector, e.g., as described in Anderson et al., U.S. Pat. No. 5,399,346; Mann et al., Cell 33:153, 1983; Temin et al., U.S. Pat. No. 4,650,764; Temin et al., U.S. Pat. No. 4,980,289; Markowitz et al., J. Virol. 62:1120, 1988; Temin et al., U.S. Pat. No. 5,124,263; Dougherty et al., WIPO Publication WO 95/07358; and Kuo et al., Blood 82:845-52, 1993.

Alternatively, the vector can be introduced by lipofection in vivo using liposomes. Synthetic cationic lipids can be used to prepare liposomes for in vivo transfection of a gene encoding a marker (Felgner et al., Proc. Natl. Acad. Sci. USA 84:7413-7, 1987; and Mackey et al., Proc. Natl. Acad. Sci. USA 85:8027-31, 1988). The use of lipofection to introduce exogenous genes into specific organs in vivo has certain practical advantages. Molecular targeting of liposomes to specific cells represents one area of benefit. It is clear that directing transfection to particular cells represents one area of benefit. It is clear that directing transfection to particular cell types would be particularly advantageous in a tissue with cellular heterogeneity, such as the pancreas, liver, kidney, and brain. Lipids may be chemically coupled to other molecules for the purpose of targeting. Targeted peptides, e.g., hormones or neurotransmitters, and proteins such as antibodies, or non-peptide molecules could be coupled to liposomes chemically.

It is possible to remove the cells from the body and introduce the vector as a naked DNA plasmid and then re-implant the transformed cells into the body. Naked DNA vector for gene therapy can be introduced into the desired host cells by methods known in the art, e.g., transfection, electroporation, microinjection, transduction, cell fusion, DEAE dextran, calcium phosphate precipitation, use of a gene gun or use of a DNA vector transporter (see, for example, Wu et al., J. Biol. Chem. 267:963-7, 1992; Wu et al., J. Biol. Chem. 263:14621-4, 1988).

Another aspect of the present invention involves antisense polynucleotide compositions that are complementary to a segment of the polynucleotide set forth in SEQ ID NO:1. Such synthetic antisense oligonucleotides are designed to bind to mRNA encoding ZSMF-16 polypeptides and to inhibit translation of such mRNA. Such antisense oligonucleotides are used to inhibit expression of ZSMF-16 polypeptide-encoding genes in cell culture or in a subject.

The present invention also provides reagents which will find use in diagnostic applications. For example, the ZSMF-16 gene, a probe comprising ZSMF-16 DNA or RNA or a subsequence thereof can be used to determine if the ZSMF-16 gene is present on a human chromosome, such as chromosome 3, or if a mutation has occurred. Based on annotation of a fragment of human genomic DNA containing the ZSMF-16 genomic DNA (Genbank Accession No. AC006208), ZSMF-16 is located at the 3p21 region of chromosome 3. Detectable chromosomal aberrations at the ZSMF-16 gene locus include, but are not limited to, aneuploidy, gene copy number changes, translocations, insertions, deletions, restriction site changes and rearrangements. Such aberrations can be detected using polynucleotides of the present invention by employing molecular genetic techniques, such as restriction fragment length polymorphism (RFLP) analysis, short tandem repeat (STR) analysis employing PCR techniques, and other genetic linkage analysis techniques known in the art (Sambrook et al., ibid.; Ausubel et. al., ibid.; Marian, Chest 108:255-65, 1995).

The precise knowledge of a gene's position can be useful for a number of purposes, including: 1) determining if a sequence is part of an existing contig and obtaining additional surrounding genetic sequences in various forms, such as YACs, BACs or cDNA clones; 2) providing a possible candidate gene for an inheritable disease which shows linkage to the same chromosomal region; and 3) cross-referencing model organisms, such as mouse, which may aid in determining what function a particular gene might have.

The ZSMF-16 gene is located at the 3p21 region of chromosome 3. Several known semaphorins map to this locus and are associated with human disease states: Semaphorins 3F, IV and A(V) map to 3p21.3 and modified expression and deletions are associated with small cell lung cancer (See, Sekido, Y et al., Proc. Natl. Acad. Sci. 93:4120-4125, 1996; Xiang, R. -H. et al., Genomics 32:39-48, 1996). ZSMF-16 polynucleotide probes can be used to detect abnormalities or genotypes associated with these semaphorin cancer susceptibility markers. Moreover, there is evidence for cancer resulting from mutations in the 3p21 region: the GNAI2 gene (3p21) may be associated with cancers, such as growth hormone secreting (ghs+) pituitary tumors (Lyons, J. et al., Science 249:655-659, 1990; Williamson, E. A. et al., Europe. J. Clin. Invest. 25:128-131, 1995), and is also associated with a type of ulcerative colitis and heart arrhythmias. Moreover, ZSMF-16 polynucleotide probes can be used to detect abnormalities or genotypes associated with the cancer susceptibility marker for colorectal cancer heredetary non-polyposis, type 2, localized to 3p21.3 (Lindblom, A. et al., Nature Genet. 5:279-282, 1993). Moreover, ZSMF-16 is expressed in breast tumor tissue. Because there is abundant evidence for cancer resulting from mutations in the 3p21.3 region, and ZSMF-16 also maps to this chromosomal locus, mutations in ZSMF-16 may also be directly involved in or associated with cancers, such as small cell lung cancer or other tumors, such as breast tumors.

Moreover, ZSMF-16 polynucleotide probes can be used to detect abnormalities or genotypes associated with the autosomal dominant degenerative neurologic disease, spinocerebellar ataxia 7, which maps to the 3p21.1-pl2 region of chromosome 3 (Benomar, A. et al., Ann. Neurol. 35:439-444, 1994; Gouw, L. G. et al., Nature Genet. 10:89-93, 1995; and Holmburg, M. et al., Hum. Molec. Genet. 4:1441-1445, 1995). A diagnostic could assist physicians in determining the type of spinocerebellar ataxia disease and appropriate associated therapy, or assistance in genetic counselling. As such, the inventive anti-ZSMF-16 antibodies, polynucleotides, and polypeptides can be used for the detection of ZSMF-16 polypeptide, niRNA or anti-ZSMF-16 antibodies, thus serving as markers and be directly used for detecting or diagnosing spinocerebellar ataxia or cancers, as described herein, using methods known in the art and described herein. Further, ZSMF-16 polynucleotide probes can be used to detect abnormalities or genotypes associated with chromosome 3p21 deletions and translocations associated with human diseases, such as renal cell carcinoma (RCC) (deletion, loss of heterozygosity, or translocation between 8q24 and 3p21), involved with malignant progression of renal tumors; catenin, beta-i (CTNNB1) (3p22-p21.3) mutations, which are expected to be involved in chromosome rearrangements in malignancy; or in other cancers. Similarly, ZSMF-16 polynucleotide probes can be used to detect abnormalities or genotypes associated with chromosome 3p21 trisomy and chromosome loss associated with human diseases such as Larsen Syndrome (3p21.1-pl4) and RCC (above). One of skill in the art would recognize that 3p21 chromosomal aberrations such as loss of heterogeneity (LOH), trisomy, rearrangements and translocations are common in several human cancers, and as such ZSMF-16 polynucleotide probes would be useful in diagnosing and detecting such cancerous tissues and genomic aberrations associated therewith. Moreover, amongst other genetic loci, those for carnitine-acylcarnitine translocase deficiency, (3p21.31), parathyroid hormone receptor 1 (PTHR1) mutations of which are associated with thyroid disease and metaphyseal chondrodysplasia (3p22-p21.1), and others, all manifest themselves in human disease states as well as map to this region of the human genome. See the Online Mendellian Inheritance of Man (OMIM™, National Center for Biotechnology Information, National Library of Medicine. Bethesda, Md.) gene map, and references therein, for this region of chromosome 3 on a publicly available WWW server (http://www3.ncbi.nlm.nih.gov/htbin-post/Omim/getmap?chromosome=3p21, and surrounding regions through 3p21.9). All of these serve as possible candidate genes for an inheritable disease which show linkage to the same chromosomal region as the ZSMF-16 gene. Thus, ZSMF-16 polynucleotide probes can be used to detect abnormalities or genotypes associated with these defects.

Similarly, defects in the ZSMF-16 gene itself may result in a heritable human disease state. The ZSMF-16 gene (3p21) is located near other semaphorins involved in human disease, as discussed above, suggesting that this chromosomal region is commonly regulated.. Moreover, one of skill in the art would appreciate that defects in semaphorins are known to cause disease states in humans. Thus, similarly, defects in ZSMF-16 can cause a disease state or susceptibility to disease. As, ZSMF-16 is a cytokine receptor in a chromosomal hot spot for aberrations involved in numerous cancers and is shown to be expressed in breast cancer cells, the molecules of the present invention could also be directly involved in cancer formation or metastasis. As the ZSMF-16 gene is located at the 3p21 region ZSMF-16, polynucleotide probes can be used to detect chromosome 3p21 loss, trisomy, duplication or translocation associated with human diseases, such as mammary tumor tissue, breast tumor and diseased breast tissues, liver, small intestine, bone, brain or other cancers, or diseases. Moreover, molecules of the present invention, such as the polypeptides, antagonists, agonists, polynucleotides and antibodies of the present invention would aid in the detection, diagnosis prevention, and treatment associated with a ZSMF-16 genetic defect.

A diagnostic could assist physicians in determining the type of disease and appropriate associated therapy, or assistance in genetic counseling. As such, the inventive anti-ZSMF-16 antibodies, polynucleotides, and polypeptides can be used for the detection of ZSMF-16 polypeptide, mRNA or anti-ZSMF-16 antibodies, thus serving as markers and be directly used for detecting or genetic diseases or cancers, as described herein, using methods known in the art and described herein. Further, ZSMF-16 polynucleotide probes can be used to detect abnormalities or genotypes associated with chromosome 3p21 deletions and translocations associated with human diseases, other translocations involved with malignant progression of tumors or other 3p21 mutations, which are expected to be involved in chromosome rearrangements in malignancy; or in other cancers, or in spontaneous abortion. Similarly, ZSMF-16 polynucleotide probes can be used to detect abnormalities or genotypes associated with chromosome 3p21 trisomy and chromosome loss associated with human diseases. Thus, ZSMF-16 polynucleotide probes can be used to detect abnormalities or genotypes associated with these defects.

As discussed above, defects in the ZSMF-16 gene itself may result in a heritable human disease state. Molecules of the present invention, such as the polypeptides, antagonists, agonists, polynucleotides and antibodies of the present invention would aid in the detection, diagnosis prevention, and treatment associated with a ZSMF-16 genetic defect. In addition, ZSMF-16 polynucleotide probes can be used to detect allelic differences between diseased or non-diseased individuals at the ZSMF-16 chromosomal locus. As such, the ZSMF-16 sequences can be used as diagnostics in forensic DNA profiling.

In general, the diagnostic methods used in genetic linkage analysis, to detect a genetic abnormality or aberration in a patient, are known in the art. Analytical probes will be generally at least 20 nt in length, although somewhat shorter probes can be used (e.g., 14-17 nt). PCR primers are at least 5 nt in length, preferably 15 or more, more preferably 20-30 nt. For gross analysis of genes, or chromosomal DNA, a ZSMF-16 polynucleotide probe may comprise an entire exon or more. Exons are readily determined by one of skill in the art by comparing ZSMF-16 sequences (SEQ ID NO:1) with the human genomic DNA for ZSMF-16 (Genbank Accession No. AC006208). In general, the diagnostic methods used in genetic linkage analysis, to detect a genetic abnormality or aberration in a patient, are known in the art. Most diagnostic methods comprise the steps of (a) obtaining a genetic sample from a potentially diseased patient, diseased patient or potential non-diseased carrier of a recessive disease allele; (b) producing a first reaction product by incubating the genetic sample with a ZSMF-16 polynucleotide probe wherein the polynucleotide will hybridize to complementary polynucleotide sequence, such as in RFLP analysis or by incubating the genetic sample with sense and antisense primers in a PCR reaction under appropriate PCR reaction conditions; (iii) Visualizing the first reaction product by gel electrophoresis and/or other known method such as visualizing the first reaction product with a ZSMF-16 polynucleotide probe wherein the polynucleotide will hybridize to the complementary polynucleotide sequence of the first reaction; and (iv) comparing the visualized first reaction product to a second control reaction product of a genetic sample from wild type patient. A difference between the first reaction product and the control reaction product is indicative of a genetic abnormality in the diseased or potentially diseased patient, or the presence of a heterozygous recessive carrier phenotype for a non-diseased patient, or the presence of a genetic defect in a tumor from a diseased patient, or the presence of a genetic abnormality in a fetus or pre-implantation embryo. For example, a difference in restriction fragment pattern, length of PCR products, length of repetitive sequences at the ZSMF-16 genetic locus, and the like, are indicative of a genetic abnormality, genetic aberration, or allelic difference in comparison to the normal wild type control. Controls can be from unaffected family members, or unrelated individuals, depending on the test and availability of samples. Genetic samples for use within the present invention include genomic DNA, MRNA, and cDNA isolated form any tissue or other biological sample from a patient, such as but not limited to, blood, saliva, semen, embryonic cells, amniotic fluid, and the like. The polynucleotide probe or primer can be RNA or DNA, and will comprise a portion of SEQ ID NO: 1, the complement of SEQ ID NO: 1, or an RNA equivalent thereof. Such methods of showing genetic linkage analysis to human disease phenotypes are well known in the art. For reference to PCR based methods in diagnostics see see, generally, Mathew (ed.), Protocols in Human Molecular Genetics (Humana Press, Inc. 1991), White (ed.), PCR Protocols: Current Methods and Applications (Humana Press, Inc. 1993), Cotter (ed.), Molecular Diagnosis of Cancer (Humana Press, Inc. 1996), Hanausek and Walaszek (eds.), Tumor Marker Protocols (Humana Press, Inc. 1998), Lo (ed.), Clinical Applications of PCR (Humana Press, Inc. 1998), and Meltzer (ed.), PCR in Bioanalysis (Humana Press, Inc. 1998)).

Aberrations associated with the ZSMF-16 locus can be detected using nucleic acid molecules of the present invention by employing standard methods for direct mutation analysis, such as restriction fragment length polymorphism analysis, short tandem repeat analysis employing PCR techniques, amplification-refractory mutation system analysis, single-strand conformation polymorphism detection, RNase cleavage methods, denaturing gradient gel electrophoresis, fluorescence-assisted mismatch analysis, and other genetic analysis techniques known in the art (see, for example, Mathew (ed.), Protocols in Human Molecular Genetics (Humana Press, Inc. 1991), Marian, Chest 108:255 (1995), Coleman and Tsongalis, Molecular Diagnostics (Human Press, Inc. 1996), Elles (ed.) Molecular Diagnosis of Genetic Diseases (Humana Press, Inc. 1996), Landegren (ed.), Laboratory Protocols for Mutation Detection (Oxford University Press 1996), Birren et al. (eds.), Genome Analysis, Vol. 2: Detecting Genes (Cold Spring Harbor Laboratory Press 1998), Dracopoli et al. (eds.), Current Protocols in Human Genetics (John Wiley & Sons 1998), and Richards and Ward, “Molecular Diagnostic Testing,” in Principles of Molecular Medicine, pages 83-88 (Humana Press, Inc. 1998)). Direct analysis of an ZSMF-16 gene for a mutation can be performed using a subject's genomic DNA. Methods for amplifying genomic DNA, obtained for example from peripheral blood lymphocytes, are well-known to those of skill in the art (see, for example, Dracopoli et al. (eds.), Current Protocols in Human Genetics, at pages 7.1.6 to 7.1.7 (John Wiley & Sons 1998)).

Mice engineered to express the ZSMF-16 gene, referred to as “transgenic mice,” and mice that exhibit a complete absence of ZSMF-16 gene function, referred to as “knockout mice,” may also be generated (Snouwaert et al., Science 257:1083, 1992; Lowell et al., Nature 366:740-42, 1993; Capecchi, M. R., Science 244: 1288-1292, 1989; Palmiter, R. D. et al. Annu Rev Genet. 20: 465-499, 1986). For example, transgenic mice that over-express ZSMF-16, either ubiquitously or under a tissue-specific or tissue-restricted promoter can be used to ask whether over-expression causes a phenotype. For example, over-expression of a wild-type ZSMF-16 polypeptide, polypeptide fragment or a mutant thereof may alter normal cellular processes, resulting in a phenotype that identifies a tissue in which ZSMF-16 expression is functionally relevant and may indicate a therapeutic target for the ZSMF-16, its agonists or antagonists. For example, a preferred transgenic mouse to engineer is one that over-expresses the mature human ZSMF-16 polypeptide (residue 23 (Gly) to residue 779 (Thr) of SEQ ID NO:2). Moreover, such over-expression may result in a phenotype that shows similarity with human diseases. Similarly, knockout ZSMF-16 mice can be used to determine where ZSMF-16 is absolutely required in vivo. The phenotype of knockout mice is predictive of the in vivo effects of that a ZSMF-16 antagonist, such as those described herein, may have. The murine ZSMF-16 mRNA, and cDNA can be isolated and used to isolate mouse ZSMF-16 genomic DNA, which are subsequently used to generate knockout mice. These transgenic and knockout mice may be employed to study the ZSMF-16 gene and the protein encoded thereby in an in vivo system, and can be used as in vivo models for corresponding human or animal diseases (such as those in commercially viable animal populations). The mouse models of the present invention are particularly relevant as tumor models for the study of cancer biology and progression. Such models are useful in the development and efficacy of therapeutic molecules used in human cancers. Because increases in ZSMF-16 expression, as well as decreases in ZSMF-16 expression are associated with specific human cancers, both transgenic mice and knockout mice would serve as useful animal models for cancer. Moreover, in a preferred embodiment, ZSMF-16 transgenic mouse can serve as an animal model for specific tumors, particularly breast cancer. Moreover, transgenic mice expression of ZSMF-16 antisense polynucleotides or ribozymes directed against ZSMF-16, described herein, can be used analogously to transgenic mice described above.

For pharmaceutical use, the proteins of the present invention are formulated for parenteral, particularly intravenous or subcutaneous, delivery according to conventional methods. Intravenous administration will be by bolus injection or infusion over a typical period of one to several hours. In general, pharmaceutical formulations will include a ZSMF-16 polypeptide in combination with a pharmaceutically acceptable vehicle, such as saline, buffered saline, 5% dextrose in water or the like. Formulations may further include one or more excipients, preservatives, solubilizers, buffering agents, albumin to prevent protein loss on vial surfaces, etc. Methods of formulation are well known in the art and are disclosed, for example, in Remington: The Science and Practice of Pharmacy, Gennaro, ed., Mack Publishing Co., Easton, Pa., 19th ed., 1995. Determination of dose is within the level of ordinary skill in the art.

The invention is further illustrated by the following non-limiting examples.

### EXAMPLES

### Example 1

### Identification of ZSMF-16

Novel ZSMF-16 encoding polynucleotides and polypeptides of the present invention were initially identified by querying an EST database for sequences homologous to conserved motifs within the semaphorin family. Expressed sequence tags (ESTs) from human breast tumor, bone, and human brain caudate/putamen/nucleus accumbens cDNA libraries were identified. In addition human genomic sequences (Genbank Accession No. AC006208) were identified that upon further analysis contained exons to complete the full length sequence. The resulting 2340 bp sequence is disclosed in SEQ ID NO:1. The full length novel semaphorin was designated ZSMF-16.

### Example 2

### Tissue Distribution

Human Multiple Tissue Northern Blots (MTN I, MTN II, and MTN III; Clontech) are probed to determine the tissue distribution of human ZSMF-16 expression. A probe is amplified from a human breast tumor or brain derived Marathon™-ready cDNA library (Clontech). Oligonucleotide primers are designed based on the EST sequence or cDNA sequence (SEQ ID NO:1; Example 1). The Marathon™-ready cDNA library is prepared according to manufacturer's instructions (Marathon™ cDNA Amplification Kit; Clontech) using human retina poly A+ RNA (Clontech). The probe is amplified in a polymerase chain reaction under reaction conditions, for example, as follows: 1 cycle at 94° C. for 1 minute; 35 cycles of 94° C. for 30 seconds and 68° C. for 1 minute 30 seconds; followed by 1 cycle at 72° C. for 10 minutes; followed by a 4° C. soak. The resulting DNA fragment is electrophoresed on an approximately 2% low melt agarose gel (SEA PLAQUE GTG low melt agarose, FMC Corp., Rockland, Me.), the fragment is purified using the QIAquick™ method (Qiagen, Chatsworth, Calif.), and the sequence is confirmed by sequence analysis.

The probe is radioactively labeled and purified as described herein using methods known in the art. ExpressHyb™ (Clontech) solution, or similar hybridization solution, is used for prehybridization and as a hybridizing solution for the Northern blots. Hybridization takes place overnight at 65° C. using about 1.0×10⁶ cpm/ml of labeled probe. The blots are then washed about 4 times at room temperature in 2X SSC, 0.05% SDS followed by about 2 washes at 50° C. in 0.1X SSC, 0.01% SDS for about 20 minutes each. A transcript of approximately 2.0-4.0 kb should be seen in tissues that express the ZSMF-16 mRNA.

Additional analysis can be carried out on Northern blots made with poly(A) RNA from the human vascular cell lines HUVEC (human umbilical vein endothelial cells; Cascade Biologics, Inc., Portland, Oreg.), HPAEC (human pulmonary artery endothelial cells; Cascade Biologics, Inc.), HAEC (human aortic endothelial cells; Cascade Biologics, Inc.), AoSMC (aortic smooth muscle cells; Clonetics, San Diego, Calif.), UASMC (umbilical artery smooth muscle cells; Clonetics), HISM (human intestinal smooth muscle cells; ATCC CRL 7130), SK-5 (human dermal fibroblast cells; obtained from Dr. Russell Ross, University of Washington, Seattle, Wash.), NHLF (normal human lung fibroblast cells; Clonetics), and NHDF-NEO (normal human dermal fibroblast-neonatal cells; Clonetics). The probe is prepared and labeled and prehybridization and hybridization were carried out essentially as disclosed above. The blots are then washed at about 50° C. in 0.1X SSC, 0.05% SDS. A transcript of approximately 2.0-4.0 kb should be seen in those cells that express the ZSMF-16 mRNA.

Additional analysis can be carried out on Northern blots made with poly(A) RNA from K-562 cells (erythroid, ATCC CCL 243), HUT78 cells (T cell, ATCC TIB-161), Jurkat cells (T cell), DAUDI (Burkitt's human lymphoma, Clontech, Palo Alto, Calif.), RAJI (Burkitt's human lymphoma, Clontech) and HL60 (Monocyte). The probe preparation and hybridization are carried out as above. A transcript of approximately 2.0-4.0 kb should be seen in those cells that express the ZSMF-16 mRNA.

Additional analysis can be carried out on Northern blots made with poly (A) RNA from CD4⁺, CD8⁺, CD19⁺ and mixed lymphocyte reaction cells (CellPro, Bothell, Wash.) using probes and hybridization conditions described above. A transcript of approximately 2.0-4.0 kb should be seen in those cells that express the ZSMF-16 mRNA.

Additional analysis can be carried out on Human Brain Multiple Tissue Northern Blots II and III (Clontech) using the probe and hybridization conditions described above. A transcript of approximately 2.0-4.0 kb should be seen in those cells that express the ZSMF-16 mRNA.

Moreover a Dot Blot is also performed using Human RNA Master Blots™ (Clontech). The methods and conditions for the Dot Blot were the same as for the Multiple Tissue Blots disclosed above. Again, a signal is present for those tissues that express the ZSMF-16 mRNA.

### Example 3

### Chromosomal Assignment and Placement of ZSMF-16

ZSMF-16 was mapped to chromosome 3 using the commercially available “GeneBridge 4 Radiation Hybrid Panel” (Research Genetics, Inc., Huntsville, Ala.). The GeneBridge 4 Radiation Hybrid Panel contains PCRable DNAs from each of 93 radiation hybrid clones, plus two control DNAs (the HFL donor and the A23 recipient). A publicly available WWW server (e.g., Center for Genome Research at the Whitehead Institute for Biomedical Research, Cambridge, Mass., USA; http://www-genome.wi.mit.edu/cgi-bin/contig/rhmapper.pl) allows mapping relative to the Whitehead Institute/MIT Center for Genome Research's radiation hybrid map of the human genome (the “WICGR” radiation hybrid map) which was constructed with the GeneBridge 4 Radiation Hybrid Panel.

For the mapping of ZSMF-16 with the “GeneBridge 4 RH Panel”, 20 μl reactions were set up in a 96-well microtiter plate compatible for PCR (Stratagene, La Jolla, Calif.) and used in a “RoboCycler Gradient 96” thermal cycler (Stratagene). Each of the 95 PCR reactions consisted of 2 μl 10X KlenTaq PCR reaction buffer (CLONTECH Laboratories, Inc., Palo Alto, Calif.), 1.6 μl dNTPs mix (2.5 mM each, PERKIN-ELMER, Foster City, Calif.), 1 μl sense primer, ZC26,039 (SEQ ID NO:5), 1 μl antisense primer, ZC26,040 (SEQ ID NO:6), 2 μl “RediLoad” (Research Genetics, Inc., Huntsville, Ala.), 0.4 μl 50X Advantage KlenTaq Polymerase Mix (Clontech Laboratories, Inc.), 25 ng of DNA from an individual hybrid clone or control and distilled water for a total volume of 20 μl. The reactions were overlaid with an equal amount of mineral oil and sealed. The PCR cycler conditions were as follows: an initial 1 cycle 5 minute denaturation at 95° C., 40 cycles of a 1 minute denaturation at 95° C., 1 minute annealing at 70° C. and 1.5 minute extension at 72° C., followed by a final 1 cycle extension of 7 minutes at 72° C. The reactions were separated by electrophoresis on a 2% agarose gel (EM Science, Gibbstown, N.J.) and visualized by staining with ethidium bromide.

The results showed that ZSMF-16 maps 4.81 cR_{—}3000 distal from the framework marker WI-9590 on the chromosome 3 WICGR radiation hybrid map. The use of surrounding genes/markers positions ZSMF-16 in the 3p21 chromosomal region.

From the foregoing, it will be appreciated that, although specific embodiments of the invention have been described herein for purposes of illustration, various modifications may be made without deviating from the spirit and scope of the invention. Accordingly, the invention is not limited except as by the appended claims.

What is claimed is:

1. An isolated polynucleotide that encodes a semaphorin polypeptide comprising a sequence of amino acid residues that is at least 90% identical to an amino acid sequence selected from the group consisting of:
(a) the amino acid sequence as shown in SEQ ID NO:2 from amino acid number 23 (Gly), to amino acid number 500 (Arg);
(b) the amino acid sequence as shown in SEQ ID NO:2 from amino acid number 76 (Leu), to amino acid number 500 (Arg);
(c) the amino acid sequence as shown in SEQ ID NO:2 from amino acid number 76 (Leu), to amino acid number 592 (Glu););
(d) the amino acid sequence as shown in SEQ ID NO:2 from amino acid number 76 (Leu), to amino acid number 654 (Thr);
(e) the amino acid sequence as shown in SEQ ID NO:2 from amino acid number 76 (Leu), to amino acid number 779 (Thr);
(f) the amino acid sequence as shown in SEQ ID NO:2 from amino acid number 23 (Gly), to amino acid number 779 (Thr); and
(g) the amino acid sequence as shown in SEQ ID NO:2 from amino acid number 1 (Met), to amino acid number 779 (Thr).

2. An isolated polynucleotide according to claim 1, wherein the polynucleotide is selected from the group consisting of:
(a) a polynucleotide sequence as shown in SEQ ID NO:1 from nucleotide 67 to nucleotide 1500;
(b) a polynucleotide sequence as shown in SEQ ID NO:1 from nucleotide 226 to nucleotide 1500;
(c) a polynucleotide sequence as shown in SEQ ID NO:1 from nucleotide 226 to nucleotide 1776; and
(d) a polynucleotide sequence as shown in SEQ ID NO:1 from nucleotide 226 to nucleotide 1961;
(e) a polynucleotide sequence as shown in SEQ ID NO:1 from nucleotide 226 to nucleotide 2337;
(f) a polynucleotide sequence as shown in SEQ ID NO:1 from nucleotide 67 to nucleotide 2337; and
(g) a polynucleotide sequence as shown in SEQ ID NO:1 from nucleotide 1 to nucleotide 2337.

3. An isolated polynucleotide sequence according to claim 1, wherein the polynucleotide comprises nucleotide 1 to nucleotide 2337 of SEQ ID NO:3.

4. An isolated polynucleotide according to claim 1, wherein the polynucleotide encodes a semaphorin polypeptide that comprises a sequence of amino acid residues selected from the group consisting of:
(a) the amino acid sequence as shown in SEQ ID NO:2 from amino acid number 23 (Gly), to amino acid number 500 (Arg);
(b) the amino acid sequence as shown in SEQ ID NO:2 from amino acid number 76 (Leu), to amino acid number 500 (Arg);
(c) the amino acid sequence as shown in SEQ ID NO:2 from amino acid number 76 (Leu), to amino acid number 592 (Glu););
(d) the amino acid sequence as shown in SEQ ID NO:2 from amino acid number 76 (Leu), to amino acid number 654 (Thr);
(e) the amino acid sequence as shown in SEQ ID NO:2 from amino acid number 76 (Leu), to amino acid number 779 (Thr);
(f) the amino acid sequence as shown in SEQ ID NO:2 from amino acid number 23 (Gly), to amino acid number 779 (Thr); and
(g) the amino acid sequence as shown in SEQ ID NO:2 from amino acid number 1 (Met), to amino acid number 779 (Thr).

5. An expression vector comprising the following operably linked elements:
a transcription promoter;
a DNA segment encoding a semaphorin polypeptide as shown in SEQ ID NO:2 from amino acid number 23 (Gly), to amino acid number 779 (Thr); and
a transcription terminator,
wherein the promoter is operably linked to the DNA segment, and the DNA segment is operably linked to the transcription terminator.

6. An expression vector according to claim 5, further comprising a secretory signal sequence operably linked to the DNA segment.

7. A cultured cell comprising an expression vector according to claim 5, wherein the cell expresses a polypeptide encoded by the DNA segment.

8. A DNA construct encoding a fusion protein, the DNA construct comprising:
a first DNA segment encoding a polypeptide comprising a sequence of amino acid residues selected from the group consisting of:
(a) the amino acid sequence as shown in SEQ ID NO:2 from amino acid number 1 (Met), to amino acid number 22 (Ser);
(b) the amino acid sequence as shown in SEQ ID NO:2 from amino acid number 23 (Gly), to amino acid number 75 (Asn);
(c) the amino acid sequence as shown in SEQ ID NO:2 from amino acid number 76 (Leu), to amino acid number 500 (Arg);
(d) the amino acid sequence as shown in SEQ ID NO:2 from amino acid number 501 (Gln), to amino acid number 592 (Glu);
(e) the amino acid sequence as shown in SEQ ID NO:2 from amino acid number 593 (His), to amino acid number 654 (Thr);
(f) the amino acid sequence as shown in SEQ ID NO:2 from amino acid number 655 (Leu), to amino acid number 779 (Thr);
(g) the amino acid sequence as shown in SEQ ID NO:2 from amino acid number 23 (Gly), to amino acid number 779 (Thr); and
at least one other DNA segment encoding an additional polypeptide,
wherein the first and other DNA segments are connected in-frame; and
wherein the first and other DNA segments encode the fusion protein.

9. An expression vector comprising the following operably linked elements:
a transcription promoter;
a DNA construct encoding a fusion protein according to claim 8; and
a transcription terminator,
wherein the promoter is operably linked to the DNA construct, and the DNA construct is operably linked to the transcription terminator.

10. A cultured cell comprising an expression vector according to claim 9, wherein the cell expresses a polypeptide encoded by the DNA construct.

11. A method of producing a fusion protein comprising:
culturing a cell according to claim 10; and
isolating the polypeptide produced by the cell.

12. An isolated semaphorin polypeptide comprising a sequence of amino acid residues that is at least 90% identical to an amino acid sequence selected from the group consisting of:
(a) the amino acid sequence as shown in SEQ ID NO:2 from amino acid number 23 (Gly), to amino acid number 500 (Arg);
(b) the amino acid sequence as shown in SEQ ID NO:2 from amino acid number 76 (Leu), to amino acid number 500 (Arg);
(c) the amino acid sequence as shown in SEQ ID NO:2 from amino acid number 76 (Leu), to amino acid number 592 (Glu););
(d) the amino acid sequence as shown in SEQ ID NO:2 from amino acid number 76 (Leu), to amino acid number 654 (Thr);
(e) the amino acid sequence as shown in SEQ ID NO:2 from amino acid number 76 (Leu), to amino acid number 779 (Thr);
(f) the amino acid sequence as shown in SEQ ID NO:2 from amino acid number 23 (Gly), to amino acid number 779 (Thr); and
(g) the amino acid sequence as shown in SEQ ID NO:2 from amino acid number 1 (Met), to amino acid number 779 (Thr).

13. An isolated polypeptide according to claim 12, wherein the polypeptide comprises a sequence of amino acid residues selected from the group consisting of:
(a) the amino acid sequence as shown in SEQ ID NO:2 from amino acid number 23 (Gly), to amino acid number 500 (Arg);
(b) the amino acid sequence as shown in SEQ ID NO:2 from amino acid number 76 (Leu), to amino acid number 500 (Arg);
(c) the amino acid sequence as shown in SEQ ID NO:2 from amino acid number 76 (Leu), to amino acid number 592 (Glu););
(d) the amino acid sequence as shown in SEQ ID NO:2 from amino acid number 76 (Leu), to amino acid number 654 (Thr);
(e) the amino acid sequence as shown in SEQ ID NO:2 from amino acid number 76 (Leu), to amino acid number 779 (Thr);
(f) the amino acid sequence as shown in SEQ ID NO:2 from amino acid number 23 (Gly), to amino acid number 779 (Thr); and
(g) the amino acid sequence as shown in SEQ ID NO:2 from amino acid number 1 (Met), to amino acid number 779 (Thr).

14. A method of producing a semaphorin polypeptide comprising:
culturing a cell according to claim 7; and
isolating the semaphorin polypeptide produced by the cell.

15. A method of producing an antibody comprising:
inoculating an animal with a polypeptide selected from the group consisting of:
(a) a polypeptide consisting of 9 to 19 amino acids, wherein the polypeptide is identical to a contiguous sequence of amino acids in SEQ ID NO:2 from amino acid number 39 (Gly) to amino acid number 57 (Tyr);
(b) a polypeptide according to claim 13;
(c) a polypeptide consisting of the amino acid sequence of SEQ ID NO:2 from amino acid number 39 (Gly) to 57 (Tyr);
(d) a polypeptide consisting of the amino acid sequence of SEQ ID NO:2 from amino acid number 107 (Asp) to 114 (Ala) of SEQ ID NO:2; and
wherein the polypeptide elicits an immune response in the animal to produce the antibody; and
isolating the antibody from the animal.

16. An antibody produced by the method of claim 15, which specifically binds to a polypeptide of SEQ ID NO:2.

17. The antibody of claim 16, wherein the antibody is a monoclonal antibody.

18. An antibody that specifically binds to a polypeptide of claim 12.

19. An antibody that specifically binds to a polypeptide of claim 13.

20. A method of detecting, in a test sample, the presence of a modulator of ZSMF-16 protein activity, comprising:
transfecting a ZSMF-16-responsive cell, with a reporter gene construct that is responsive to a ZSMF-16-stimulated cellular pathway; and
producing a ZSMF-16 polypeptide by the method of claim 14; and
adding the ZSMF-16 polypeptide to the cell, in the presence and absence of a test sample; and
comparing levels of response to the ZSMF-16 polypeptide, in the presence and absence of the test sample, by a biological or biochemical assay; and
determining from the comparison, the presence of the modulator of ZSMF-16 activity in the test sample.

21. A method for detecting a genetic abnormality in a patient, comprising:
obtaining a genetic sample from a patient;
producing a first reaction product by incubating the genetic sample with a polynucleotide comprising at least 14 contiguous nucleotides of SEQ ID NO:1 or the complement of SEQ ID NO:1, under conditions wherein said polynucleotide will hybridize to complementary polynucleotide sequence;
visualizing the first reaction product; and
comparing said first reaction product to a control reaction product from a wild type patient, wherein a difference between said first reaction product and said control reaction product is indicative of a genetic abnormality in the patient.

22. A method for detecting a cancer in a patient, comprising:
obtaining a tissue or biological sample from a patient;
incubating the tissue or biological sample with an antibody of claim 19 under conditions wherein the antibody binds to its complementary polypeptide in the tissue or biological sample;
visualizing the antibody bound in the tissue or biological sample; and
comparing levels of antibody bound in the tissue or biological sample from the patient to a normal control tissue or biological sample,
wherein an increase or decrease in the level of antibody bound to the patient tissue or biological sample relative to the normal control tissue or biological sample is indicative of a cancer in the patient.

23. A method for detecting a cancer in a patient, comprising:
obtaining a tissue or biological sample from a patient;
labeling a polynucleotide comprising at least 14 contiguous nucleotides of SEQ ID NO:1 or the complement of SEQ ID NO:1;
incubating the tissue or biological sample with under conditions wherein the polynucleotide will hybridize to complementary polynucleotide sequence;
visualizing the labeled polynucleotide in the tissue or biological sample; and
comparing the level of labeled polynucleotide hybridization in the tissue or biological sample from the patient to a normal control tissue or biological sample,
wherein an increase or decrease in the labeled polynucleotide hybridization to the patient tissue or biological sample relative to the normal control tissue or biological sample is indicative of a cancer in the patient.


SEQUENCE LISTING (6 sequences)

SEQ ID NO: 1 - DNA, 2340 bp, Homo Sapiens
  CDS (1)...(2340)
atggccccctcggcctgggccatttgctggctgctagggggcctcctgctccatgggggtagctctggccccagccccggccccagtgtgccccgcctgcggctctcctaccgaggagccatggtccgaaagccttccagcaccatgtggatggaaacattttccagatacctcctgtctgccaaccgctctgccatctttctgggcccccagggctccctgaacctccaggccatgtacctagatgagtaccgagaccgcctctttctgggtggcctggacgccctctactctctgcggctggaccaggcatggccagatccccgggagacagagtgcgccaacttcgtgcgggtgctacagcctcacaaccggacccacctgctagcctgtggcactggggccttccagcccacctgtgccctcatcacagttggccaccgtggggagcatgtgctccacctggagcctggcagtgtggaaagtggccgggggcggtgccctcacgagcccagccgtccctttgccagcaccttcatagacggggagctgtacacgggtctcactgctgacttcctggggcgagaggccatgatcttccgaagtggaggtcctcggccagctctgcgttccgactctgaccagagtctcttgcacgacccccggtttgtgatggccgcccggatccctgagaactctgaccaggacaatgacaaggtgtacttcttcttctcggagacggtcccctcgcccgatggtggctcgaaccatgtcactgtcagccgcgtgggccgcgtctgcgtgaatgatgctgggggccagcgggtgctggtgaacaaatggagcactttcctcaaggccaggctggtctgctcggtgcccggccctggtggtgccgagacccactttgaccagctagaggatgtgttcctgctgtggcccaaggccgggaagagcctcgaggtgtacgcgctgttcagcaccgtcagtgccgtgttccagggcttcgccgtctgtgtgtaccacatggcagacatctgggaggttttcaacgggccctttgcccaccgagatgggcctcagcaccagtgggggccctatgggggcaaggtgcccttccctcgccctggcgtgtgccccagcaagatgaccgcacagccaggacggccttttggcagcaccaaggactacccagatgaggtgctgcagtttgcccgagcccaccccctcatgttctggcctgtgcggcctcgacatggccgccctgtccttgtcaagacccacctggcccagcagctacaccagatcgtggtggaccgcgtggaggcagaggatgggacctacgatgtcattttcctggggactgactcagggtctgtgctcaaagtcatcgctctccaggcagggggctcagctgaacctgaggaagtggttctggaggagctccaggtgtttaaggtgccaacacctatcaccgaaatggagatctctgtcaaaaggcaaatgctatacgtgggctctcggctgggtgtggcccagctgcggctgcaccaatgtgagacttacggcactgcctgtgcagagtgctgcctggcccgggacccatactgtgcctgggatggtgcctcctgtacccactaccgccccagccttggcaagcgccggttccgccggcaggacatccggcacggcaaccctgccctgcagtgcctgggccagagccaggaagaagaggcagtgggacttgtggcagccaccatggtctacggcacggagcacaatagcaccttcctggagtgcctgcccaagtctccccaggctgctgtgcgctggctcttgcagaggccaggggatgaggggcctgaccaggtgaagacggacgagcgagtcttgcacacggagcgggggctgctgttccgcaggcttagccgtttcgatgcgggcacctacacctgcaccactctggagcatggcttctcccagactgtggtccgcctggctctggtggtgattgtggcctcacagctggacaacctgttccctccggagccaaagccagaggagcccccagcccggggaggcctggcttccaccccacccaaggcctggtacaaggacatcctgcagctcattggcttcgccaacctgccccgggtggatgagtactgtgagcgcgtgtggtgcaggggcaccacggaatgctcaggctgcttccggagccggagccggggcaagcaggccaggggcaagagctgggcagggctggagctaggcaagaagatgaagagccgggtgcatgccgagcacaatcggacgccccgggaggtggaggccacgtag

SEQ ID NO: 2 - protein, 779 aa, Homo Sapiens
MAPSAWAICWLLGGLLLHGGSSGPSPGPSVPRLRLSYRGAMVRKPSSTMWMETFSRYLLSANRSAIFLGPQGSLNLQAMYLDEYRDRLFLGGLDALYSLRLDQAWPDPRETECANFVRVLQPHNRTHLLACGTGAFQPTCALITVGHRGEHVLHLEPGSVESGRGRCPHEPSRPFASTFIDGELYTGLTADFLGREAMIFRSGGPRPALRSDSDQSLLHDPRFVMAARIPENSDQDNDKVYFFFSETVPSPDGGSNHVTVSRVGRVCVNDAGGQRVLVNKWSTFLKARLVCSVPGPGGAETHFDQLEDVFLLWPKAGKSLEVYALFSTVSAVFQGFAVCVYHMADIWEVFNGPFAHRDGPQHQWGPYGGKVPFPRPGVCPSKMTAQPGRPFGSTKDYPDEVLQFARAHPLMFWPVRPRHGRPVLVKTHLAQQLHQIVVDRVEAEDGTYDVIFLGTDSGSVLKVIALQAGGSAEPEEVVLEELQVFKVPTPITEMEISVKRQMLYVGSRLGVAQLRLHQCETYGTACAECCLARDPYCAWDGASCTHYRPSLGKRRFRRQDIRHGNPALQCLGQSQEEEAVGLVAATMVYGTEHNSTFLECLPKSPQAAVRWLLQRPGDEGPDQVKTDERVLHTERGLLFRRLSRFDAGTYTCTTLEHGFSQTVVRLALVVIVASQLDNLFPPEPKPEEPPARGGLASTPPKAWYKDILQLIGFANLPRVDEYCERVWCRGTTECSGCFRSRSRGKQARGKSWAGLELGKKMKSRVHAEHNRTPREVEAT

SEQ ID NO: 3 - DNA, 2337 bp, Artificial Sequence
  Degenerate Polynucleotide sequence of ZSMF-16
atggcnccnwsngcntgggcnathtgytggytnytnggnggnytnytnytncayggnggnwsnwsnggnccnwsnccnggnccnwsngtnccnmgnytnmgnytnwsntaymgnggngcnatggtnmgnaarccnwsnwsnacnatgtggatggaracnttywsnmgntayytnytnwsngcnaaymgnwsngcnathttyytnggnccncarggnwsnytnaayytncargcnatgtayytngaygartaymgngaymgnytnttyytnggnggnytngaygcnytntaywsnytnmgnytngaycargcntggccngayccnmgngaracngartgygcnaayttygtnmgngtnytncarccncayaaymgnacncayytnytngcntgyggnacnggngcnttycarccnacntgygcnytnathacngtnggncaymgnggngarcaygtnytncayytngarccnggnwsngtngarwsnggnmgnggnmgntgyccncaygarccnwsnmgnccnttygcnwsnacnttyathgayggngarytntayacnggnytnacngcngayttyytnggnmgngargcnatgathttymgnwsnggnggnccnmgnccngcnytnmgnwsngaywsngaycarwsnytnytncaygayccnmgnttygtnatggcngcnmgnathccngaraaywsngaycargayaaygayaargtntayttyttyttywsngaracngtnccnwsnccngayggnggnwsnaaycaygtnacngtnwsnmgngtnggnmgngtntgygtnaaygaygcnggnggncarmgngtnytngtnaayaartggwsnacnttyytnaargcnmgnytngtntgywsngtnccnggnccnggnggngcngaracncayttygaycarytngargaygtnttyytnytntggccnaargcnggnaarwsnytngargtntaygcnytnttywsnacngtnwsngcngtnttycarggnttygcngtntgygtntaycayatggcngayathtgggargtnttyaayggnccnttygcncaymgngayggnccncarcaycartggggnccntayggnggnaargtnccnttyccnmgnccnggngtntgyccnwsnaaratgacngcncarccnggnmgnccnttyggnwsnacnaargaytayccngaygargtnytncarttygcnmgngcncayccnytnatgttytggccngtnmgnccnmgncayggnmgnccngtnytngtnaaracncayytngcncarcarytncaycarathgtngtngaymgngtngargcngargayggnacntaygaygtnathttyytnggnacngaywsnggnwsngtnytnaargtnathgcnytncargcnggnggnwsngcngarccngargargtngtnytngargarytncargtnttyaargtnccnacnccnathacngaratggarathwsngtnaarmgncaratgytntaygtnggnwsnmgnytnggngtngcncarytnmgnytncaycartgygaracntayggnacngcntgygcngartgytgyytngcnmgngayccntaytgygcntgggayggngcnwsntgyacncaytaymgnccnwsnytnggnaarmgnmgnttymgnmgncargayathmgncayggnaayccngcnytncartgyytnggncarwsncargargargargcngtnggnytngtngcngcnacnatggtntayggnacngarcayaaywsnacnttyytngartgyytnccnaarwsnccncargcngcngtnmgntggytnytncarmgnccnggngaygarggnccngaycargtnaaracngaygarmgngtnytncayacngarmgnggnytnytnttymgnmgnytnwsnmgnttygaygcnggnacntayacntgyacnacnytngarcayggnttywsncaracngtngtnmgnytngcnytngtngtnathgtngcnwsncarytngayaayytnttyccnccngarccnaarccngargarccnccngcnmgnggnggnytngcnwsnacnccnccnaargcntggtayaargayathytncarytnathggnttygcnaayytnccnmgngtngaygartaytgygarmgngtntggtgymgnggnacnacngartgywsnggntgyttymgnwsnmgnwsnmgnggnaarcargcnmgnggnaarwsntgggcnggnytngarytnggnaaraaratgaarwsnmgngtncaygcngarcayaaymgnacnccnmgngargtngargcnacn

SEQ ID NO: 4 - protein, 7 aa, Artificial Sequence
  Glu-Glu tag
EEYMPME

SEQ ID NO: 5 - DNA, 18 bp, Artificial Sequence
  Oligonucleotide primer ZC26039
cagggtctgtgctcaaag

SEQ ID NO: 6 - DNA, 18 bp, Artificial Sequence
  Oligonucleotide primer ZC26040
tttcggtgataggtgttg